SEQUENCE LISTING (247 sequences)

SEQ ID NO: 1 - protein, 20 aa, Artificial Sequence
  Description of Artificial Sequence Synthetic peptide
GGGGSGGGGSGGGGSGGGGS

SEQ ID NO: 2 - protein, 20 aa, Artificial Sequence
  Description of Artificial Sequence Synthetic peptide
GKPGSGKPGSGKPGSGKPGS

SEQ ID NO: 3 - protein, 25 aa, Artificial Sequence
  Description of Artificial Sequence Synthetic peptide
GGGGSGGGGSGGGGSGGGGSGGGGS

SEQ ID NO: 4 - protein, 18 aa, Artificial Sequence
  Description of Artificial Sequence Synthetic peptide
GSTSGSGKPGSGEGSTKG

SEQ ID NO: 5 - protein, 14 aa, Artificial Sequence
  Description of Artificial Sequence Synthetic peptide
IRPRAIGGSKPRVA

SEQ ID NO: 6 - protein, 15 aa, Artificial Sequence
  Description of Artificial Sequence Synthetic peptide
GKGGSGKGGSGKGGS

SEQ ID NO: 7 - protein, 15 aa, Artificial Sequence
  Description of Artificial Sequence Synthetic peptide
GGKGSGGKGSGGKGS

SEQ ID NO: 8 - protein, 15 aa, Artificial Sequence
  Description of Artificial Sequence Synthetic peptide
GGGKSGGGKSGGGKS

SEQ ID NO: 9 - protein, 15 aa, Artificial Sequence
  Description of Artificial Sequence Synthetic peptide
GKGKSGKGKSGKGKS

SEQ ID NO: 10 - protein, 15 aa, Artificial Sequence
  Description of Artificial Sequence Synthetic peptide
GGGKSGGKGSGKGGS

SEQ ID NO: 11 - protein, 15 aa, Artificial Sequence
  Description of Artificial Sequence Synthetic peptide
GKPGSGKPGSGKPGS

SEQ ID NO: 12 - protein, 20 aa, Artificial Sequence
  Description of Artificial Sequence Synthetic peptide
GKGKSGKGKSGKGKSGKGKS

SEQ ID NO: 13 - protein, 20 aa, Artificial Sequence
  Description of Artificial Sequence Synthetic peptide
GGGGSGGGGSGGGGSGGGGS

SEQ ID NO: 14 - protein, 14 aa, Artificial Sequence
  Description of Artificial Sequence Synthetic peptide
STAGDTHLGGEDFD

SEQ ID NO: 15 - protein, 15 aa, Artificial Sequence
  Description of Artificial Sequence Synthetic peptide
GEGGSGEGGSGEGGS

SEQ ID NO: 16 - protein, 15 aa, Artificial Sequence
  Description of Artificial Sequence Synthetic peptide
GGEGSGGEGSGGEGS

SEQ ID NO: 17 - protein, 15 aa, Artificial Sequence
  Description of Artificial Sequence Synthetic peptide
GGGESGGGESGGGES

SEQ ID NO: 18 - protein, 15 aa, Artificial Sequence
  Description of Artificial Sequence Synthetic peptide
GEGESGEGESGEGES

SEQ ID NO: 19 - protein, 15 aa, Artificial Sequence
  Description of Artificial Sequence Synthetic peptide
GGGESGGEGSGEGGS

SEQ ID NO: 20 - protein, 20 aa, Artificial Sequence
  Description of Artificial Sequence Synthetic peptide
GEGESGEGESGEGESGEGES

SEQ ID NO: 21 - protein, 19 aa, Artificial Sequence
  Description of Artificial Sequence Synthetic peptide
PRGASKSGSASQTGSAPGS

SEQ ID NO: 22 - protein, 19 aa, Artificial Sequence
  Description of Artificial Sequence Synthetic peptide
GTAAAGAGAAGGAAAGAAG

SEQ ID NO: 23 - protein, 19 aa, Artificial Sequence
  Description of Artificial Sequence Synthetic peptide
GTSGSSGSGSGGSGSGGGG

SEQ ID NO: 24 - protein, 5 aa, Artificial Sequence
  Description of Artificial Sequence Synthetic peptide
GGGGS

SEQ ID NO: 25 - protein, 10 aa, Artificial Sequence
  Description of Artificial Sequence Synthetic peptide
GGGGSGGGGS

SEQ ID NO: 26 - protein, 30 aa, Artificial Sequence
  Description of Artificial Sequence Synthetic polypeptide
GGGGSGGGGSGGGGSGGGGSGGGGSGGGGS

SEQ ID NO: 27 - protein, 35 aa, Artificial Sequence
  Description of Artificial Sequence Synthetic polypeptide
GGGGSGGGGSGGGGSGGGGSGGGGSGGGGSGGGGS

SEQ ID NO: 28 - protein, 30 aa, Artificial Sequence
  Description of Artificial Sequence Synthetic polypeptide
DPALVHQRPAPPGGGGSGGGGSGGGGSGGG

SEQ ID NO: 29 - protein, 5 aa, Artificial Sequence
  Description of Artificial Sequence Synthetic peptide
GKPGS

SEQ ID NO: 30 - protein, 10 aa, Artificial Sequence
  Description of Artificial Sequence Synthetic peptide
GKPGSGKPGS

SEQ ID NO: 31 - protein, 25 aa, Artificial Sequence
  Description of Artificial Sequence Synthetic peptide
GKPGSGKPGSGKPGSGKPGSGKPGS

SEQ ID NO: 32 - protein, 30 aa, Artificial Sequence
  Description of Artificial Sequence Synthetic polypeptide
GKPGSGKPGSGKPGSGKPGSGKPGSGKPGS

SEQ ID NO: 33 - protein, 35 aa, Artificial Sequence
  Description of Artificial Sequence Synthetic polypeptide
GKPGSGKPGSGKPGSGKPGSGKPGSGKPGSGKPGS

SEQ ID NO: 34 - protein, 5 aa, Artificial Sequence
  Description of Artificial Sequence Synthetic peptide
GGGES

SEQ ID NO: 35 - protein, 10 aa, Artificial Sequence
  Description of Artificial Sequence Synthetic peptide
GGGESGGGES

SEQ ID NO: 36 - protein, 20 aa, Artificial Sequence
  Description of Artificial Sequence Synthetic peptide
GGGESGGGESGGGESGGGES

SEQ ID NO: 37 - protein, 25 aa, Artificial Sequence
  Description of Artificial Sequence Synthetic peptide
GGGESGGGESGGGESGGGESGGGES

SEQ ID NO: 38 - protein, 30 aa, Artificial Sequence
  Description of Artificial Sequence Synthetic polypeptide
GGGESGGGESGGGESGGGESGGGESGGGES

SEQ ID NO: 39 - protein, 35 aa, Artificial Sequence
  Description of Artificial Sequence Synthetic polypeptide
GGGESGGGESGGGESGGGESGGGESGGGESGGGES

SEQ ID NO: 40 - protein, 329 aa, Artificial Sequence
  Description of Artificial Sequence Synthetic polypeptide
  Backbone 1 Constant Heavy Domain Monomer 1 (-)
ASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 41 - protein, 329 aa, Artificial Sequence
  Description of Artificial Sequence Synthetic polypeptide
  Backbone 1 Constant Heavy Domain Monomer 2 (+)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 329 aa, Artificial Sequence; identical to SEQ ID NO: 40
  Description of Artificial Sequence Synthetic polypeptide
  Backbone 2 Constant Heavy Domain Monomer 1 (-)

SEQ ID NO: 43 - protein, 329 aa, Artificial Sequence
  Description of Artificial Sequence Synthetic polypeptide
  Backbone 2 Constant Heavy Domain Monomer 2 (+)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 329 aa, Artificial Sequence
  Description of Artificial Sequence Synthetic polypeptide
  Backbone 3 Constant Heavy Domain Monomer 1 (-)
ASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCEVSGFYPSDIAVEWESDGQPENNYKTTPPVLDSDGSFFLYSKLTVDKSRWEQGDVFSCSVMHEALHNHYTQKSLSLSPGK

SEQ ID NO: 45 - protein, 329 aa, Artificial Sequence; identical to SEQ ID NO: 43
  Description of Artificial Sequence Synthetic polypeptide
  Backbone 3 Constant Heavy Domain Monomer 2 (+)

SEQ ID NO: 46 - protein, 329 aa, Artificial Sequence
  Description of Artificial Sequence Synthetic polypeptide
  Backbone 4 Constant Heavy Domain Monomer 1 (-)
ASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTENEVSLTCLVKGFYPSDIAVEWESDGQPENNYKTTPPVLDSDGSFFLYSKLEVDKSRWEQGDVFSCSVMHEALHNHYTQKSLSLSPGK

SEQ ID NO: 47 - protein, 329 aa, Artificial Sequence
  Description of Artificial Sequence Synthetic polypeptide
  Backbone 4 Constant Heavy Domain Monomer 2 (+)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSKGSFFLYSKLTVDKSRWQQGNVFSCSVMHEALHNHYTQKSLSLSPGK

SEQ ID NO: 48 - protein, 329 aa, Artificial Sequence
  Description of Artificial Sequence Synthetic polypeptide
  Backbone 5 Constant Heavy Domain Monomer 1 (-)
ASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSRDELTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 49 - protein, 329 aa, Artificial Sequence
  Description of Artificial Sequence Synthetic polypeptide
  Backbone 5 Constant Heavy Domain Monomer 2 (+)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSRDQLTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 329 aa, Artificial Sequence
  Description of Artificial Sequence Synthetic polypeptide
  Backbone 6 Constant Heavy Domain Monomer 1 (-)
ASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYASTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 51 - protein, 329 aa, Artificial Sequence
  Description of Artificial Sequence Synthetic polypeptide
  Backbone 6 Constant Heavy Domain Monomer 2 (+)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYASTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 329 aa, Artificial Sequence
  Description of Artificial Sequence Synthetic polypeptide
  Backbone 7 Constant Heavy Domain Monomer 1 (-)
ASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYSSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 53 - protein, 329 aa, Artificial Sequence
  Description of Artificial Sequence Synthetic polypeptide
  Backbone 7 Constant Heavy Domain Monomer 2 (+)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYSSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 327 aa, Artificial Sequence
  Description of Artificial Sequence Synthetic polypeptide
  Backbone 8 Constant Heavy Domain Monomer 1 (-)
ASTKGPSVFPLAPCSRSTSESTAALGCLVKDYFPEPVTVSWNSGALTSGVHTFPAVLQSSGLYSLSSVVTVPSSSLGTKTYTCNVDHKPSDTKVDKRVESKYGPPCPPCPAPEFLGGPSVFLFPPKPKDTLMISRTPEVTCVVVDVSQEDPEVQFNWYVDGVEVHNAKTKPREEEFNSTYRVVSVLTVLHQDWLNGKEYKCKVSNKGLPSSIEKTISKAKGQPREPQVYTLPPSQEEMTKNQVSLTCDVSGFYPSDIAVEWESDGQPENNYKTTPPVLDSDGSFFLYSRLTVDKSRWEEGDVFSCSVMHEALHNHYTQKSLSLSLGK

SEQ ID NO: 55 - protein, 327 aa, Artificial Sequence
  Description of Artificial Sequence Synthetic polypeptide
  Backbone 8 Constant Heavy Domain Monomer 2 (+)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEQMTKNQVKLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 56 - protein, 326 aa, Artificial Sequence
  Description of Artificial Sequence Synthetic polypeptide
  Backbone 9 Constant Heavy Domain Monomer 1 (-)
ASTKGPSVFPLAPCSRSTSESTAALGCLVKDYFPEPVTVSWNSGALTSGVHTFPAVLQSSGLYSLSSVVTVPSSNFGTQTYTCNVDHKPSDTKVDKTVERKCCVECPPCPAPPVAGPSVFLFPPKPKDTLMISRTPEVTCVVVDVSHEDPEVQFNWYVDGVEVHNAKTKPREEEFNSTFRVVSVLTVVHQDWLNGKEYKCKVSNKGLPAPIEKTISKTKGQPREPQVYTLPPSREEMTKNQVSLTCDVSGFYPSDIAVEWESDGQPENNYKTTPPMLDSDGSFFLYSKLTVDKSRWEQGDVFSCSVMHEALHNHYTQKSLSLSPGK

SEQ ID NO: 57 - protein, 326 aa, Artificial Sequence
  Description of Artificial Sequence Synthetic polypeptide
  Backbone 9 Constant Heavy Domain Monomer 2 (+)
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREQMTKNQVKLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 58 - protein, 326 aa, Artificial Sequence
  Description of Artificial Sequence Synthetic polypeptide
  Backbone 10 Constant Heavy Domain Monomer 1 (-)
ASTKGPSVFPLAPCSRSTSESTAALGCLVKDYFPEPVTVSWNSGALTSGVHTFPAVLQSSGLYSLSSVVTVPSSNFGTQTYTCNVDHKPSDTKVDKTVERKCCVECPPCPAPPVAGPSVFLFPPKPKDTLMISRTPEVTCVVVDVKHEDPEVQFNWYVDGVEVHNAKTKPREEEFNSTFRVVSVLTVVHQDWLNGKEYKCKVSNKGLPAPIEKTISKTKGQPREPQVYTLPPSREEMTKNQVSLTCDVSGFYPSDIAVEWESDGQPENNYKTTPPMLDSDGSFFLYSKLTVDKSRWEQGDVFSCSVMHEALHNHYTQKSLSLSPGK

SEQ ID NO: 59 - protein, 326 aa, Artificial Sequence
  Description of Artificial Sequence Synthetic polypeptide
  Backbone 10 Constant Heavy Domain Monomer 2 (+)
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVKHEDPEVQFNWYVDGVEVHNAKTKPREEQFNSTFRVVSVLTVVHQDWLNGKEYKCKVSNKGLPAPIEKTISKTKGQPREPQVYTLPPSREQMTKNQVKLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 60 - protein, 329 aa, Artificial Sequence
  Description of Artificial Sequence Synthetic polypeptide
  Backbone 11 Constant Heavy Domain Monomer 1 (-)
ASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 61 - protein, 329 aa, Artificial Sequence
  Description of Artificial Sequence Synthetic polypeptide
  Backbone 11 Constant Heavy Domain Monomer 2 (+)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 62 - protein, 329 aa, Artificial Sequence
  Description of Artificial Sequence Synthetic polypeptide
  Backbone 12 Constant Heavy Domain Monomer 1 (-)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCDVSGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 329 aa, Artificial Sequence
  Description of Artificial Sequence Synthetic polypeptide
  Backbone 12 Constant Heavy Domain Monomer 2 (+)
ASTKGPSVFPLAPSSKSTSGGTAALGCLVKDYFPEPVTVSWNSGALTSGVHTFPAVLQSSGLYSLSSVVTVPSSSLGTKTYTCNVNHKPSNTKVDKKVERKSSDKTHTCPPRPAPPVAGPSVFLFPPKPKDTLMISRTPEVTCVVVDVKHEDPEVKFKWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 107 aa, Artificial Sequence [sequence omitted]

SEQ ID NO: 65 - protein, 106 aa, Artificial Sequence
  Description of Artificial Sequence Synthetic polypeptide
  Constant Light Domain - Lambda
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 66 - protein, 460 aa, Artificial Sequence
  Description of Artificial Sequence Synthetic polypeptide
  scClambdaCH or scCL(lambda)CH
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSGGGGSGGGGSGGGGSGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 461 aa, Artificial Sequence
  Description of Artificial Sequence Synthetic polypeptide
  scCkappaCH or scCL(kappa)CH
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 460 aa, Artificial Sequence
  Description of Artificial Sequence Synthetic polypeptide
  scClambdaCH(A118C) or scCL(lambda)CH(A118C)
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSGGGGSGGGGSGGGGSGGGGSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 461 aa, Artificial Sequence
  Description of Artificial Sequence Synthetic polypeptide
  scCkappaCH(A118C) or scCL(kappa)CH(A118C)
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 450 aa, Artificial Sequence
  Description of Artificial Sequence Synthetic polypeptide
  XENP13677 [alphaCD20]_H1.202_IgG1_PVA_/ S267K_pI(-)_Isosteric_A_L368D/K370S
QVQLVQSGAEVKKPGASVKVSCKASGYTFTSYNMHWVRQAPGQRLEWMGAIYPGNGATSYSQKFQGRVTITADTSASTAYMELSSLRSEDTAVYYCARSYYMGGDWYFDVWGAGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 71 - protein, 485 aa, Artificial Sequence
  Description of Artificial Sequence Synthetic polypeptide
  XENP13677 [alphaCD3]_H1.30_L1.47_scFv(GKPGS) 4_Fc(216)_IgG1_C220S/S364K/E357Q
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 213 aa, Artificial Sequence
  Description of Artificial Sequence Synthetic polypeptide
  XENP13677 [aCD20]_L1.113 Light Chain
QIVLTQSPSSLSASVGDRVTITCRASWSVSYIHWFQQKPGKSPKPLIYATSNLASGVPVRFSGSGSGTDYTLTISSLQPEDFATYYCQQWTHNPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 453 aa, Artificial Sequence
  Description of Artificial Sequence Synthetic polypeptide
  XENP023535 [alphaFAP]_H0L0_Fab_IgG1_pI (-)_Isosteric_A_/PVA_/S267K/L368D/K370S Fab-Fc Heavy Chain
QVQLVQSGAEVKKPGASVKVSCKTSRYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 74 - protein, 485 aa, Artificial Sequence; identical to SEQ ID NO: 71
  Description of Artificial Sequence Synthetic polypeptide
  XENP023535 [alphaCD3]_H1.30_L1.47_scFv(GKPGS) 4_Fc(216)_IgG1_C220S/PVA_/S267K/S364K/E357Q scFv-Fc Heavy Chain

SEQ ID NO: 75 - protein, 220 aa, Artificial Sequence
  Description of Artificial Sequence Synthetic polypeptide
  XENP023535 [alphaFAP]_L0 Light Chain
DIVMTQSPDSLAVSLGERATINCKSSQSLLYSRNQKNYLAWYQQKPGQPPKLLIFWASTRESGVPDRFSGSGFGTDFTLTISSLQAEDVAVYYCQQYFSYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 76 - protein, 449 aa, Artificial Sequence
  Description of Artificial Sequence Synthetic polypeptide
  XENP27982 [alphaCD123]_H1.109_IgG1_pI(-)_ Isosteric_A_/PVA_/S267K/L368D/K370S/M428L/N434S
QVQLQQSGAEVKKPGASVKVSCKASGYTFTDYYMKWVKQSHGKSLEWMGDIIPSNGATFYNQKFKGKATLTVDRSTSTAYMELSSLRSEDTAVYYCARSHLLRASWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 77 - protein, 485 aa, Artificial Sequence
  Description of Artificial Sequence Synthetic polypeptide
  XENP27982 [alphaCD3]_H1.30_L1.47_scFv(GKPGS) 4_C220S/PVA_/S267K/S364K/E357Q/M428L/N434S
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 78 - protein, 220 aa, Artificial Sequence
  Description of Artificial Sequence Synthetic polypeptide
  XENP27982 [alphaCD123]_L1.57 Light Chain
DFVMTQSPDSLAVSLGERATINCKSSQSLLNTGNQKNYLTWYQQKPGQPPKLLIYWASTRESGVPDRFTGSGSGTDFTLTISSLQAEDVAVYYCQNDYSYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 79 - protein, 254 aa, Artificial Sequence
  Description of Artificial Sequence Synthetic polypeptide
  Anti-CD3-High - [anti-CD3]_H1.30_L1.47_scFv scFv
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 80 - protein, 125 aa, Artificial Sequence
  Description of Artificial Sequence Synthetic polypeptide
  Anti-CD3-High - [anti-CD3]_H1.30_L1.47_scFv Variable Heavy (vh) Domain
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSS

SEQ ID NO: 81 - protein, 5 aa, Artificial Sequence
  Description of Artificial Sequence Synthetic peptide
  Anti-CD3-High - [anti-CD3]_H1.30_L1.47_scFv vhCDR1
TYAMN

SEQ ID NO: 82 - protein, 19 aa, Artificial Sequence
  Description of Artificial Sequence Synthetic peptide
  Anti-CD3-High - [anti-CD3]_H1.30_L1.47_scFv vhCDR2
RIRSKYNNYATYYADSVKG

SEQ ID NO: 83 - protein, 14 aa, Artificial Sequence
  Description of Artificial Sequence Synthetic peptide
  Anti-CD3-High - [anti-CD3]_H1.30_L1.47_scFv vhCDR3
HGNFGDSYVSWFAY

SEQ ID NO: 84 - protein, 109 aa, Artificial Sequence
  Description of Artificial Sequence Synthetic polypeptide
  Anti-CD3-High - [anti-CD3]_H1.30_L1.47_scFv Variable Light (vl) Domain
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 85 - protein, 14 aa, Artificial Sequence
  Description of Artificial Sequence Synthetic peptide
  Anti-CD3-High - [anti-CD3]_H1.30_L1.47_scFv vlCDR1
GSSTGAVTTSNYAN

SEQ ID NO: 86 - protein, 7 aa, Artificial Sequence
  Description of Artificial Sequence Synthetic peptide
  Anti-CD3-High - [anti-CD3]_H1.30_L1.47_scFv vlCDR2
GTNKRAP

SEQ ID NO: 87 - protein, 9 aa, Artificial Sequence
  Description of Artificial Sequence Synthetic peptide
  Anti-CD3-High - [anti-CD3]_H1.30_L1.47_scFv vlCDR3
ALWYSNHWV

SEQ ID NO: 88 - protein, 20 aa, Artificial Sequence
  Description of Artificial Sequence Synthetic peptide
  Anti-CD3-High - [anti-CD3]_H1.30_L1.47_scFv Linker
GKPGSGKPGSGKPGSGKPGS

SEQ ID NO: 89 - protein, 254 aa, Artificial Sequence
  Description of Artificial Sequence Synthetic polypeptide
  Anti-CD3-High-Int - [anti-CD3]_H1.32_L1.47_ scFv scFv
EVQLVESGGGLVQPGGSLRLSCAASGFTFSTYAMNWVRQAPGKGLEWVGRIRSKANNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 90 - protein, 125 aa, Artificial Sequence
  Description of Artificial Sequence Synthetic polypeptide
  Anti-CD3-High-Int - [anti-CD3]_H1.32_L1.47_ scFv Variable Heavy (vh) Domain
EVQLVESGGGLVQPGGSLRLSCAASGFTFSTYAMNWVRQAPGKGLEWVGRIRSKANNYATYYADSVKGRFTISRDDSKNTLYLQMNSLRAEDTAVYYCVRHGNFGDSYVSWFAYWGQGTLVTVSS

SEQ ID NO: 91 - protein, 5 aa, Artificial Sequence
  Description of Artificial Sequence Synthetic peptide
  Anti-CD3-High-Int - [anti-CD3]_H1.32_L1.47_ scFv vhCDR1
TYAMN

SEQ ID NO: 92 - protein, 19 aa, Artificial Sequence
  Description of Artificial Sequence Synthetic peptide
  Anti-CD3-High-Int - [anti-CD3]_H1.32_L1.47_ scFv vhCDR2
RIRSKANNYATYYADSVKG

SEQ ID NO: 93 - protein, 14 aa, Artificial Sequence
  Description of Artificial Sequence Synthetic peptide
  Anti-CD3-High-Int - [anti-CD3]_H1.32_L1.47_ scFv vhCDR3
HGNFGDSYVSWFAY

SEQ ID NO: 94 - protein, 109 aa, Artificial Sequence; identical to SEQ ID NO: 84
  Description of Artificial Sequence Synthetic polypeptide
  Anti-CD3-High-Int - [anti-CD3]_H1.32_L1.47_ scFv Variable Light (vl) Domain

SEQ ID NO: 95 - protein, 14 aa, Artificial Sequence
  Description of Artificial Sequence Synthetic peptide
  Anti-CD3-High-Int - [anti-CD3]_H1.32_L1.47_ scFv vlCDR1
GSSTGAVTTSNYAN

SEQ ID NO: 96 - protein, 7 aa, Artificial Sequence
  Description of Artificial Sequence Synthetic peptide
  Anti-CD3-High-Int - [anti-CD3]_H1.32_L1.47_ scFv vlCDR2
GTNKRAP

SEQ ID NO: 97 - protein, 9 aa, Artificial Sequence
  Description of Artificial Sequence Synthetic peptide
  Anti-CD3-High-Int - [anti-CD3]_H1.32_L1.47_ scFv vlCDR3
ALWYSNHWV

SEQ ID NO: 98 - protein, 20 aa, Artificial Sequence
  Description of Artificial Sequence Synthetic peptide
  Anti-CD3-High-Int - [anti-CD3]_H1.32_L1.47_ scFv Linker
GKPGSGKPGSGKPGSGKPGS

SEQ ID NO: 99 - protein, 254 aa, Artificial Sequence
  Description of Artificial Sequence Synthetic polypeptide
  Anti-CD3-Intermediate - [anti-CD3]_H1.33_ L1.47_scFv scFv
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFDYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 100 - protein, 125 aa, Artificial Sequence
  Description of Artificial Sequence Synthetic polypeptide
  Anti-CD3-Intermediate - [anti-CD3]_H1.33_ L1.47_scFv Variable Heavy (vh) Domain
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFDYWGQGTLVTVSS

SEQ ID NO: 101 - protein, 5 aa, Artificial Sequence
  Description of Artificial Sequence Synthetic peptide
  Anti-CD3-Intermediate - [anti-CD3]_H1.33_ L1.47_scFv vhCDR1
TYAMN

SEQ ID NO: 102 - protein, 19 aa, Artificial Sequence
  Description of Artificial Sequence Synthetic peptide
  Anti-CD3-Intermediate - [anti-CD3]_H1.33_ L1.47_scFv vhCDR2
RIRSKYNNYATYYADSVKG

SEQ ID NO: 103 - protein, 14 aa, Artificial Sequence
  Description of Artificial Sequence Synthetic peptide
  Anti-CD3-Intermediate - [anti-CD3]_H1.33_ L1.47_scFv vhCDR3
HGNFGDSYVSWFDY

SEQ ID NO: 104 - protein, 109 aa, Artificial Sequence; identical to SEQ ID NO: 84
  Description of Artificial Sequence Synthetic polypeptide
  Anti-CD3-Intermediate - [anti-CD3]_H1.33_ L1.47_scFv Variable Light (vl) Domain

SEQ ID NO: 105 - protein, 14 aa, Artificial Sequence
  Description of Artificial Sequence Synthetic peptide
  Anti-CD3-Intermediate - [anti-CD3]_H1.33_ L1.47_scFv vlCDR1
GSSTGAVTTSNYAN

SEQ ID NO: 106 - protein, 7 aa, Artificial Sequence
  Description of Artificial Sequence Synthetic peptide
  Anti-CD3-Intermediate - [anti-CD3]_H1.33_ L1.47_scFv vlCDR2
GTNKRAP

SEQ ID NO: 107 - protein, 9 aa, Artificial Sequence
  Description of Artificial Sequence Synthetic peptide
  Anti-CD3-Intermediate - [anti-CD3]_H1.33_ L1.47_scFv vlCDR3
ALWYSNHWV

SEQ ID NO: 108 - protein, 20 aa, Artificial Sequence
  Description of Artificial Sequence Synthetic peptide
  Anti-CD3-Intermediate - [anti-CD3]_H1.33_ L1.47_scFv Linker
GKPGSGKPGSGKPGSGKPGS

SEQ ID NO: 109 - protein, 254 aa, Artificial Sequence
  Description of Artificial Sequence Synthetic polypeptide
  Anti-CD3-High - [anti-CD3]_H1.89_L1.47_scFv scFv
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDEYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 110 - protein, 125 aa, Artificial Sequence
  Description of Artificial Sequence Synthetic polypeptide
  Anti-CD3-High - [anti-CD3]_H1.89_L1.47_scFv Variable Heavy (vh) Domain
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDEYVSWFAYWGQGTLVTVSS

SEQ ID NO: 111 - protein, 5 aa, Artificial Sequence
  Description of Artificial Sequence Synthetic peptide
  Anti-CD3-High - [anti-CD3]_H1.89_L1.47_scFv vhCDR1
TYAMN

SEQ ID NO: 112 - protein, 19 aa, Artificial Sequence
  Description of Artificial Sequence Synthetic peptide
  Anti-CD3-High - [anti-CD3]_H1.89_L1.47_scFv vhCDR2
RIRSKYNNYATYYADSVKG

SEQ ID NO: 113 - protein, 14 aa, Artificial Sequence
  Description of Artificial Sequence Synthetic peptide
  Anti-CD3-High - [anti-CD3]_H1.89_L1.47_scFv vhCDR3
HGNFGDEYVSWFAY

SEQ ID NO: 114 - protein, 109 aa, Artificial Sequence; identical to SEQ ID NO: 84
  Description of Artificial Sequence Synthetic polypeptide
  Anti-CD3-High - [anti-CD3]_H1.89_L1.47_scFv Variable Light (vl) Domain

SEQ ID NO: 115 - protein, 14 aa, Artificial Sequence
  Description of Artificial Sequence Synthetic peptide
  Anti-CD3-High - [anti-CD3]_H1.89_L1.47_scFv vlCDR1
GSSTGAVTTSNYAN

SEQ ID NO: 116 - protein, 7 aa, Artificial Sequence
  Description of Artificial Sequence Synthetic peptide
  Anti-CD3-High - [anti-CD3]_H1.89_L1.47_scFv vlCDR2
GTNKRAP

SEQ ID NO: 117 - protein, 9 aa, Artificial Sequence
  Description of Artificial Sequence Synthetic peptide
  Anti-CD3-High - [anti-CD3]_H1.89_L1.47_scFv vlCDR3
ALWYSNHWV

SEQ ID NO: 118 - protein, 20 aa, Artificial Sequence
  Description of Artificial Sequence Synthetic peptide
  Anti-CD3-High - [anti-CD3]_H1.89_L1.47_scFv Linker
GKPGSGKPGSGKPGSGKPGS

SEQ ID NO: 119 - protein, 254 aa, Artificial Sequence
  Description of Artificial Sequence Synthetic polypeptide
  Anti-CD3-High - [anti-CD3]_H1.31_L1.47_scFv scFv
EVQLVESGGGLVQPGGSLRLSCAASGFTFSTYAMSWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 120 - protein, 125 aa, Artificial Sequence
  Description of Artificial Sequence Synthetic polypeptide
  Anti-CD3-High - [anti-CD3]_H1.31_L1.47_scFv Variable Heavy (vh) Domain
EVQLVESGGGLVQPGGSLRLSCAASGFTFSTYAMSWVRQAPGKGLEWVGRIRSKYNNYATYYADSVKGRFTISRDDSKNTLYLQMNSLRAEDTAVYYCVRHGNFGDSYVSWFAYWGQGTLVTVSS

SEQ ID NO: 121 - protein, 5 aa, Artificial Sequence
  Description of Artificial Sequence Synthetic peptide
  Anti-CD3-High - [anti-CD3]_H1.31_L1.47_scFv vhCDR1
TYAMS

SEQ ID NO: 122 - protein, 19 aa, Artificial Sequence
  Description of Artificial Sequence Synthetic peptide
  Anti-CD3-High - [anti-CD3]_H1.31_L1.47_scFv vhCDR2
RIRSKYNNYATYYADSVKG

SEQ ID NO: 123 - protein, 14 aa, Artificial Sequence
  Description of Artificial Sequence Synthetic peptide
  Anti-CD3-High - [anti-CD3]_H1.31_L1.47_scFv vhCDR3
HGNFGDSYVSWFAY

SEQ ID NO: 124 - protein, 109 aa, Artificial Sequence; identical to SEQ ID NO: 84
  Description of Artificial Sequence Synthetic polypeptide
  Anti-CD3-High - [anti-CD3]_H1.31_L1.47_scFv Variable Light (vl) Domain

SEQ ID NO: 125 - protein, 14 aa, Artificial Sequence
  Description of Artificial Sequence Synthetic peptide
  Anti-CD3-High - [anti-CD3]_H1.31_L1.47_scFv vlCDR1
GSSTGAVTTSNYAN

SEQ ID NO: 126 - protein, 7 aa, Artificial Sequence
  Description of Artificial Sequence Synthetic peptide
  Anti-CD3-High - [anti-CD3]_H1.31_L1.47_scFv vlCDR2
GTNKRAP

SEQ ID NO: 127 - protein, 9 aa, Artificial Sequence
  Description of Artificial Sequence Synthetic peptide
  Anti-CD3-High - [anti-CD3]_H1.31_L1.47_scFv vlCDR3
ALWYSNHWV

SEQ ID NO: 128 - protein, 20 aa, Artificial Sequence
  Description of Artificial Sequence Synthetic peptide
  Anti-CD3-High - [anti-CD3]_H1.31_L1.47_scFv Linker
GKPGSGKPGSGKPGSGKPGS

SEQ ID NO: 129 - protein, 254 aa, Artificial Sequence
  Description of Artificial Sequence Synthetic polypeptide
  Anti-CD3-High - [anti-CD3]_H1.90_L1.47_scFv scFv
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDPYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 130 - protein, 125 aa, Artificial Sequence
  Description of Artificial Sequence Synthetic polypeptide
  Anti-CD3-High - [anti-CD3]_H1.90_L1.47_scFv Variable Heavy (vh) Domain
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDPYVSWFAYWGQGTLVTVSS

SEQ ID NO: 131 - protein, 5 aa, Artificial Sequence
  Description of Artificial Sequence Synthetic peptide
  Anti-CD3-High - [anti-CD3]_H1.90_L1.47_scFv vhCDR1
TYAMN

SEQ ID NO: 132 - protein, 19 aa, Artificial Sequence
  Description of Artificial Sequence Synthetic peptide
  Anti-CD3-High - [anti-CD3]_H1.90_L1.47_scFv vhCDR2
RIRSKYNNYATYYADSVKG

SEQ ID NO: 133 - protein, 14 aa, Artificial Sequence
  Description of Artificial Sequence Synthetic peptide
  Anti-CD3-High - [anti-CD3]_H1.90_L1.47_scFv vhCDR3
HGNFGDPYVSWFAY

SEQ ID NO: 134 - protein, 109 aa, Artificial Sequence; identical to SEQ ID NO: 84
  Description of Artificial Sequence Synthetic polypeptide
  Anti-CD3-High - [anti-CD3]_H1.90_L1.47_scFv Variable Light (vl) Domain

SEQ ID NO: 135 - protein, 14 aa, Artificial Sequence
  Description of Artificial Sequence Synthetic peptide
  Anti-CD3-High - [anti-CD3]_H1.90_L1.47_scFv vlCDR1
GSSTGAVTTSNYAN

SEQ ID NO: 136 - protein, 7 aa, Artificial Sequence
  Description of Artificial Sequence Synthetic peptide
  Anti-CD3-High - [anti-CD3]_H1.90_L1.47_scFv vlCDR2
GTNKRAP

SEQ ID NO: 137 - protein, 9 aa, Artificial Sequence
  Description of Artificial Sequence Synthetic peptide
  Anti-CD3-High - [anti-CD3]_H1.90_L1.47_scFv vlCDR3
ALWYSNHWV

SEQ ID NO: 138 - protein, 20 aa, Artificial Sequence
  Description of Artificial Sequence Synthetic peptide
  Anti-CD3-High - [anti-CD3]_H1.90_L1.47_scFv Linker
GKPGSGKPGSGKPGSGKPGS

SEQ ID NO: 139 - protein, 449 aa, Artificial Sequence
  Description of Artificial Sequence Synthetic polypeptide
  XENP28853 [alphaCD123]_H1.109_IgG1_PVA_/S267K_ pI(-)_Isosteric_A_L368D/K370S Heavy Chain
QVQLQQSGAEVKKPGASVKVSCKASGYTFTDYYMKWVKQSHGKSLEWMGDIIPSNGATFYNQKFKGKATLTVDRSTSTAYMELSSLRSEDTAVYYCARSHLLRASWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 140 - protein, 714 aa, Artificial Sequence
  Description of Artificial Sequence Synthetic polypeptide
  XENP28853 [alphaCD3]_H1.30_L1.47_scFv(GKPGS) 4_Clambda_(GGGGS)5_CH1-Fc(216)_S364K/E357Q scFv-scCLCH Chain
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSGGGGSGGGGSGGGGSGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 141 - protein, 220 aa, Artificial Sequence; identical to SEQ ID NO: 78
  Description of Artificial Sequence Synthetic polypeptide
  XENP28853 [alphaCD123]_L1.57 Light Chain

SEQ ID NO: 142 - protein, 450 aa, Artificial Sequence
  Description of Artificial Sequence Synthetic polypeptide
  XENC101 [alphaCD20]_H1_IgG1_PVA_/S267K_pI(-)_ Isosteric_A_L368D/K370S Heavy Chain
QVQLVQSGAEVKKPGASVKVSCKASGYTFTSYNMHWVRQAPGQGLEWMGAIYPGNGDTSYNQKFQGRVTITADKSISTAYMELSSLRSEDTAVYYCARSTYYGGDWYFNVWGAGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 143 - protein, 714 aa, Artificial Sequence; identical to SEQ ID NO: 140
  Description of Artificial Sequence Synthetic polypeptide
  XENC102 [alphaCD3]_H1.30_L1.47_scFv(GKPGS)4_ Clambda_(GGGGS)5_CH1-Fc(216)_S364K/E357Q scFv-scCLCH Chain

SEQ ID NO: 144 - protein, 213 aa, Artificial Sequence
  Description of Artificial Sequence Synthetic polypeptide
  XENC103 [alphaCD20]_L1 Light Chain
QIVLTQSPSSLSASVGDRVTITCRASSSVSYIHWFQQKPGKSPKPLIYATSNLASGVPVRFSGSGSGTDYTLTISSLQPEDFATYYCQQWTSNPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 145 - protein, 444 aa, Artificial Sequence
  Description of Artificial Sequence Synthetic polypeptide
  XENC105 [alphaPSMA]_H1_IgG1_PVA_/S267K_pI(-)_ Isosteric_A_L368D/K370S Heavy Chain
EVQLVQSGAEVKKPGASVKVSCKTSGYTFTEYTIHWVRQAPGQSLEWMGNINPNNGGTTYNQKFQGRVTITVDKSTSTAYMELSSLRSEDTAVYYCAAGWNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 146 - protein, 714 aa, Artificial Sequence; identical to SEQ ID NO: 140
  Description of Artificial Sequence Synthetic polypeptide
  XENC106 [alphaCD3]_H1.30_L1.47_scFv(GKPGS)4_ Clambda_(GGGGS)5_CH1-Fc(216)_S364K/E357Q scFv-scCLCH Chain

SEQ ID NO: 147 - protein, 214 aa, Artificial Sequence
  Description of Artificial Sequence Synthetic polypeptide
  XENC107 [alphaPSMA]_L1 Light Chain
DIVMTQSPDSLAVSLGERATLSCRASQDVGTAVDWYQQKPDQSPKLLIYWASTRHTGVPDRFTGSGSGTDFTLTISSLQAEDVAVYFCQQYNSYPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 148 - protein, 451 aa, Artificial Sequence
  Description of Artificial Sequence Synthetic polypeptide
  XENC109 [alphaSSTR2]_H1.143_IgG1_PVA_/S267K_ pI(-)_Isosteric_A_L368D/K370S Heavy Chain
EVQLVESGGGLVQPGGSLRLSCAASGFTFSDYGMAWFRQAPGKGLEWVSFISNLGYSIYYADSVKGRFTISRDNAKNSLYLQMNSLRAEDTAVYYCARAPYDYDSFDPMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 149 - protein, 714 aa, Artificial Sequence; identical to SEQ ID NO: 140
  Description of Artificial Sequence Synthetic polypeptide
  XENC110 [alphaCD3]_H1.30_L1.47_scFv(GKPGS) 4_Clambda_(GGGGS)5_CH1-Fc(216)_S364K/E357Q scFv-scCLCH Chain

SEQ ID NO: 150 - protein, 219 aa, Artificial Sequence
  Description of Artificial Sequence Synthetic polypeptide
  XENC111 [alphaSSTR2]_L1.30 Light Chain
DIVMTQSPDSLAVSLGERATINCKSSQSLLNSRNRKNYLAWYQQKPDQSPKLLIYWASTRESGVPDRFSGSGSGTDFTLTISSLQAEDVAVYYCKQSYYLWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 151 - protein, 254 aa, Artificial Sequence
  Description of Artificial Sequence Synthetic polypeptide
  Anti-CD3-High - [anti-CD3]_H1.30_L1.47_ (VH-S112C) scFv scFv
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVCSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 152 - protein, 125 aa, Artificial Sequence
  Description of Artificial Sequence Synthetic polypeptide
  Anti-CD3-High - [anti-CD3]_H1.30_L1.47_ (VH-S112C) scFv Variable Heavy (vh) Domain
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVCS

SEQ ID NO: 153 - protein, 5 aa, Artificial Sequence
  Description of Artificial Sequence Synthetic peptide
  Anti-CD3-High - [anti-CD3]_H1.30_L1.47_ (VH-S112C) scFv vhCDR1
TYAMN

SEQ ID NO: 154 - protein, 19 aa, Artificial Sequence
  Description of Artificial Sequence Synthetic peptide
  Anti-CD3-High - [anti-CD3]_H1.30_L1.47_ (VH-S112C) scFv vhCDR2
RIRSKYNNYATYYADSVKG

SEQ ID NO: 155 - protein, 14 aa, Artificial Sequence
  Description of Artificial Sequence Synthetic peptide
  Anti-CD3-High - [anti-CD3]_H1.30_L1.47_ (VH-S112C) scFv vhCDR3
HGNFGDSYVSWFAY

SEQ ID NO: 156 - protein, 109 aa, Artificial Sequence; identical to SEQ ID NO: 84
  Description of Artificial Sequence Synthetic polypeptide
  Anti-CD3-High - [anti-CD3]_H1.30_L1.47_ (VH-S112C) scFv Variable Light (vl) Domain

SEQ ID NO: 157 - protein, 14 aa, Artificial Sequence
  Description of Artificial Sequence Synthetic peptide
  Anti-CD3-High - [anti-CD3]_H1.30_L1.47_ (VH-S112C) scFv vlCDR1
GSSTGAVTTSNYAN

SEQ ID NO: 158 - protein, 7 aa, Artificial Sequence
  Description of Artificial Sequence Synthetic peptide
  Anti-CD3-High - [anti-CD3]_H1.30_L1.47_ (VH-S112C) scFv vlCDR2
GTNKRAP

SEQ ID NO: 159 - protein, 9 aa, Artificial Sequence
  Description of Artificial Sequence Synthetic peptide
  Anti-CD3-High - [anti-CD3]_H1.30_L1.47_ (VH-S112C) scFv vlCDR3
ALWYSNHWV

SEQ ID NO: 160 - protein, 20 aa, Artificial Sequence
  Description of Artificial Sequence Synthetic peptide
  Anti-CD3-High - [anti-CD3]_H1.30_L1.47_ (VH-S112C) scFv Linker
GKPGSGKPGSGKPGSGKPGS

SEQ ID NO: 161 - protein, 254 aa, Artificial Sequence
  Description of Artificial Sequence Synthetic polypeptide
  Anti-CD3-High - [anti-CD3]_H1.30_L1.47_ (VH-S113C) scFv scFv
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSCGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 162 - protein, 125 aa, Artificial Sequence
  Description of Artificial Sequence Synthetic polypeptide
  Anti-CD3-High - [anti-CD3]_H1.30_L1.47_ (VH-S113C) scFv Variable Heavy (vh) Domain
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSC

SEQ ID NO: 163 - protein, 5 aa, Artificial Sequence
  Description of Artificial Sequence Synthetic peptide
  Anti-CD3-High - [anti-CD3]_H1.30_L1.47_ (VH-S113C) scFv vhCDR1
TYAMN

SEQ ID NO: 164 - protein, 19 aa, Artificial Sequence
  Description of Artificial Sequence Synthetic peptide
  Anti-CD3-High - [anti-CD3]_H1.30_L1.47_ (VH-S113C) scFv vhCDR2
RIRSKYNNYATYYADSVKG

SEQ ID NO: 165 - protein, 14 aa, Artificial Sequence
  Description of Artificial Sequence Synthetic peptide
  Anti-CD3-High - [anti-CD3]_H1.30_L1.47_ (VH-S113C) scFv vhCDR3
HGNFGDSYVSWFAY

SEQ ID NO: 166 - protein, 109 aa, Artificial Sequence; identical to SEQ ID NO: 84
  Description of Artificial Sequence Synthetic polypeptide
  Anti-CD3-High - [anti-CD3]_H1.30_L1.47_ (VH-S113C) scFv Variable Light (vl) Domain

SEQ ID NO: 167 - protein, 14 aa, Artificial Sequence
  Description of Artificial Sequence Synthetic peptide
  Anti-CD3-High - [anti-CD3]_H1.30_L1.47_ (VH-S113C) scFv vlCDR1
GSSTGAVTTSNYAN

SEQ ID NO: 168 - protein, 7 aa, Artificial Sequence
  Description of Artificial Sequence Synthetic peptide
  Anti-CD3-High - [anti-CD3]_H1.30_L1.47_ (VH-S113C) scFv vlCDR2
GTNKRAP

SEQ ID NO: 169 - protein, 9 aa, Artificial Sequence
  Description of Artificial Sequence Synthetic peptide
  Anti-CD3-High - [anti-CD3]_H1.30_L1.47_ (VH-S113C) scFv vlCDR3
ALWYSNHWV

SEQ ID NO: 170 - protein, 20 aa, Artificial Sequence
  Description of Artificial Sequence Synthetic peptide
  Anti-CD3-High - [anti-CD3]_H1.30_L1.47_ (VH-S113C) scFv Linker
GKPGSGKPGSGKPGSGKPGS

SEQ ID NO: 171 - protein, 449 aa, Artificial Sequence; identical to SEQ ID NO: 139
  Description of Artificial Sequence Synthetic polypeptide
  XENP29169 [alphaCD123]_H1.109_IgG1_PVA_/S267K_ pI(-)_Isosteric_A_L368D/K370S Heavy Chain

SEQ ID NO: 172 - protein, 714 aa, Artificial Sequence
  Description of Artificial Sequence Synthetic polypeptide
  XENP29169 [alphaCD3]_H1.30_L1.47_scFv(GKPGS) 4_VH-S112C_Clambda_(GGGGS)5_CH1-Fc(216)_S364K/E357Q scFv-scCLCH Chain
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVCSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSGGGGSGGGGSGGGGSGGGGSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 173 - protein, 220 aa, Artificial Sequence; identical to SEQ ID NO: 78
  Description of Artificial Sequence Synthetic polypeptide
  XENP29169 [alphaCD123]_L1.57 Light Chain

SEQ ID NO: 174 - protein, 449 aa, Artificial Sequence; identical to SEQ ID NO: 139
  Description of Artificial Sequence Synthetic polypeptide
  XENP29170 [alphaCD123]_H1.109_L1.57_Fab_IgG1_ PVA_/S267K_pI(-)_Isosteric_A_L368D/K370S Heavy Chain

SEQ ID NO: 175 - protein, 714 aa, Artificial Sequence
  Description of Artificial Sequence Synthetic polypeptide
  XENP29170 [alphaCD3]_H1.30_L1.47_scFv(GKPGS) 4_VH-S113C_Clambda_(GGGGS)5_CH1-Fc(216) IgG1_C220S/S364K/E357Q scFv-scCLCH Chain
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSCGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSGGGGSGGGGSGGGGSGGGGSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 176 - protein, 220 aa, Artificial Sequence; identical to SEQ ID NO: 78
  Description of Artificial Sequence Synthetic polypeptide
  XENP29170 [alphaCD123]_L1.57 Light Chain

SEQ ID NO: 177 - protein, 450 aa, Artificial Sequence; identical to SEQ ID NO: 142
  Description of Artificial Sequence Synthetic polypeptide
  XENC113  [alphaCD20]_H1_IgG1_PVA_/S267K_pI(-)_ Isosteric_A_L368D/K370S Heavy Chain

SEQ ID NO: 178 - protein, 714 aa, Artificial Sequence; identical to SEQ ID NO: 172
  Description of Artificial Sequence Synthetic polypeptide
  XENC114 [alphaCD3]_H1.30_L1.47_scFv(GKPGS)4_ VH-S112C_Clambda_(GGGGS)5_CH1-Fc(216)_S364K/E357Q scFv-scCLCH Chain

SEQ ID NO: 179 - protein, 213 aa, Artificial Sequence; identical to SEQ ID NO: 144
  Description of Artificial Sequence Synthetic polypeptide
  XENC115 [alphaCD20]_L1 Light Chain

SEQ ID NO: 180 - protein, 450 aa, Artificial Sequence; identical to SEQ ID NO: 142
  Description of Artificial Sequence Synthetic polypeptide
  XENC117  [alphaCD20]_H1L1_Fab_IgG1_PVA_/S267K_ pI(-)_Isosteric_A_L368D/K370S Heavy Chain

SEQ ID NO: 181 - protein, 714 aa, Artificial Sequence; identical to SEQ ID NO: 175
  Description of Artificial Sequence Synthetic polypeptide
  XENC118  [alphaCD3]_H1.30_L1.47_scFv(GKPGS) 4_VH-S113C_Clambda_(GGGGS)5_CH1-Fc(216) IgG1_C220S/S364K/E357Q scFv-scCLCH Chain

SEQ ID NO: 182 - protein, 213 aa, Artificial Sequence; identical to SEQ ID NO: 144
  Description of Artificial Sequence Synthetic polypeptide
  XENC119  [alphaCD20]_L1 Light Chain

SEQ ID NO: 183 - protein, 450 aa, Artificial Sequence; identical to SEQ ID NO: 142
  Description of Artificial Sequence Synthetic polypeptide
  XENC121  [alphaCD20]_H1_IgG1_PVA_/S267K_pI(-)_ Isosteric_A_L368D/K370S Heavy Chain

SEQ ID NO: 184 - protein, 714 aa, Artificial Sequence
  Description of Artificial Sequence Synthetic polypeptide
  XENC122  [alphaCD3]_H1.152_L1.64_scFv(GKPGS) 4_Clambda_(GGGGS)5_CH1-Fc(216)_S364K/E357Q scFv-scCLCH Chain
EVQLVESGGGLVQPGGSLRLSCAASGFTFSTYAMNWVRQAPGKCLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGCGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSGGGGSGGGGSGGGGSGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 185 - protein, 213 aa, Artificial Sequence; identical to SEQ ID NO: 144
  Description of Artificial Sequence Synthetic polypeptide
  XENC123  [alphaCD20]_L1 Light Chain

SEQ ID NO: 186 - protein, 444 aa, Artificial Sequence; identical to SEQ ID NO: 145
  Description of Artificial Sequence Synthetic polypeptide
  XENC125 [alphaPSMA]_H1_IgG1_PVA_/S267K_pI(-)_ Isosteric_A_L368D/K370S Heavy Chain

SEQ ID NO: 187 - protein, 714 aa, Artificial Sequence; identical to SEQ ID NO: 172
  Description of Artificial Sequence Synthetic polypeptide
  XENC126 [alphaCD3]_H1.30_L1.47_scFv(GKPGS) 4_VH-S112C_Clambda_(GGGGS)5_CH1-Fc(216)_S364K/E357Q scFv-scCLCH Chain

SEQ ID NO: 188 - protein, 214 aa, Artificial Sequence; identical to SEQ ID NO: 147
  Description of Artificial Sequence Synthetic polypeptide
  XENC127 [alphaPSMA]_L1 Light Chain

SEQ ID NO: 189 - protein, 444 aa, Artificial Sequence; identical to SEQ ID NO: 145
  Description of Artificial Sequence Synthetic polypeptide
  XENCXXX [alphaPSMA]_H1L1_Fab_IgG1_PVA_/ S267K_pI(-)_Isosteric_A_L368D/K370S Heavy Chain

SEQ ID NO: 190 - protein, 714 aa, Artificial Sequence; identical to SEQ ID NO: 175
  Description of Artificial Sequence Synthetic polypeptide
  XENC129 [alphaCD3]_H1.30_L1.47_scFv(GKPGS) 4_VH-S113C_Clambda_(GGGGS)5_CH1-Fc(216) IgG1_C220S/S364K/E357Q scFv-scCLCH Chain

SEQ ID NO: 191 - protein, 214 aa, Artificial Sequence; identical to SEQ ID NO: 147
  Description of Artificial Sequence Synthetic polypeptide
  XENC130 [alphaPSMA]_L1 Light Chain

SEQ ID NO: 192 - protein, 451 aa, Artificial Sequence; identical to SEQ ID NO: 148
  Description of Artificial Sequence Synthetic polypeptide
  XENC132 [alphaSSTR2]_H1.143_IgG1_PVA_/S267K_ pI(-)_Isosteric_A_L368D/K370S Heavy Chain

SEQ ID NO: 193 - protein, 714 aa, Artificial Sequence; identical to SEQ ID NO: 172
  Description of Artificial Sequence Synthetic polypeptide
  XENC133 [alphaCD3]_H1.30_L1.47_scFv(GKPGS) 4_VH-S112C_Clambda_(GGGGS)5_CH1-Fc(216)_S364K/E357Q scFv-scCLCH Chain

SEQ ID NO: 194 - protein, 219 aa, Artificial Sequence; identical to SEQ ID NO: 150
  Description of Artificial Sequence Synthetic polypeptide
  XENC134 [alphaSSTR2]_L1.30 Light Chain

SEQ ID NO: 195 - protein, 451 aa, Artificial Sequence; identical to SEQ ID NO: 148
  Description of Artificial Sequence Synthetic polypeptide
  XENC136 [alphaSSTR2]_H1.143_L1.30_Fab_IgG1_ PVA_/S267K_pI(-)_Isosteric_A_L368D/K370S Heavy Chain

SEQ ID NO: 196 - protein, 714 aa, Artificial Sequence; identical to SEQ ID NO: 175
  Description of Artificial Sequence Synthetic polypeptide
  XENC137 [alphaCD3]_H1.30_L1.47_scFv(GKPGS) 4_VH-S113C_Clambda_(GGGGS)5_CH1-Fc(216) IgG1_C220S/S364K/E357Q scFv-scCLCH Chain

SEQ ID NO: 197 - protein, 219 aa, Artificial Sequence; identical to SEQ ID NO: 150
  Description of Artificial Sequence Synthetic polypeptide
  XENC138 [alphaSSTR2]_L1.30 Light Chain

SEQ ID NO: 198 - protein, 254 aa, Artificial Sequence
  Description of Artificial Sequence Synthetic polypeptide
  Disulfide Stabilized Anti-CD3-High - [anti-CD3]_ H1.152_L1.64_scFv scFv
EVQLVESGGGLVQPGGSLRLSCAASGFTFSTYAMNWVRQAPGKCLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGCGTKLTVL

SEQ ID NO: 199 - protein, 125 aa, Artificial Sequence
  Description of Artificial Sequence Synthetic polypeptide
  Disulfide Stabilized Anti-CD3-High - [anti-CD3]_ H1.152_L1.64_scFv Variable Heavy (vh) Domain
EVQLVESGGGLVQPGGSLRLSCAASGFTFSTYAMNWVRQAPGKCLEWVGRIRSKYNNYATYYADSVKGRFTISRDDSKNTLYLQMNSLRAEDTAVYYCVRHGNFGDSYVSWFAYWGQGTLVTVSS

SEQ ID NO: 200 - protein, 5 aa, Artificial Sequence
  Description of Artificial Sequence Synthetic peptide
  Disulfide Stabilized Anti-CD3-High - [anti-CD3]_ H1.152_L1.64_scFv vhCDR1
TYAMN

SEQ ID NO: 201 - protein, 19 aa, Artificial Sequence
  Description of Artificial Sequence Synthetic peptide
  Disulfide Stabilized Anti-CD3-High - [anti-CD3]_ H1.152_L1.64_scFv vhCDR2
RIRSKYNNYATYYADSVKG

SEQ ID NO: 202 - protein, 14 aa, Artificial Sequence
  Description of Artificial Sequence Synthetic peptide
  Disulfide Stabilized Anti-CD3-High - [anti-CD3]_ H1.152_L1.64_scFv vhCDR3
HGNFGDSYVSWFAY

SEQ ID NO: 203 - protein, 109 aa, Artificial Sequence
  Description of Artificial Sequence Synthetic polypeptide
  Disulfide Stabilized Anti-CD3-High - [anti-CD3]_ H1.152_L1.64_scFv Variable Light (vl) Domain
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGCGTKLTVL

SEQ ID NO: 204 - protein, 14 aa, Artificial Sequence
  Description of Artificial Sequence Synthetic peptide
  Disulfide Stabilized Anti-CD3-High - [anti-CD3]_ H1.152_L1.64_scFv vlCDR1
GSSTGAVTTSNYAN

SEQ ID NO: 205 - protein, 7 aa, Artificial Sequence
  Description of Artificial Sequence Synthetic peptide
  Disulfide Stabilized Anti-CD3-High - [anti-CD3]_ H1.152_L1.64_scFv vlCDR2
GTNKRAP

SEQ ID NO: 206 - protein, 9 aa, Artificial Sequence
  Description of Artificial Sequence Synthetic peptide
  Disulfide Stabilized Anti-CD3-High - [anti-CD3]_ H1.152_L1.64_scFv vlCDR3
ALWYSNHWV

SEQ ID NO: 207 - protein, 20 aa, Artificial Sequence
  Description of Artificial Sequence Synthetic peptide
  Disulfide Stabilized Anti-CD3-High - [anti-CD3]_ H1.152_L1.64_scFv Linker
GKPGSGKPGSGKPGSGKPGS

SEQ ID NO: 208 - protein, 254 aa, Artificial Sequence
  Description of Artificial Sequence Synthetic polypeptide
  Disulfide Stabilized Anti-CD3-High-Int#1 - [anti-CD3]_ H1.153_L1.64_scFv scFv
EVQLVESGGGLVQPGGSLRLSCAASGFTFSTYAMNWVRQAPGKCLEWVGRIRSKANNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGCGTKLTVL

SEQ ID NO: 209 - protein, 125 aa, Artificial Sequence
  Description of Artificial Sequence Synthetic polypeptide
  Disulfide Stabilized Anti-CD3-High-Int#1 - [anti-CD3]_ H1.153_L1.64_scFv Variable Heavy (vh) Domain
EVQLVESGGGLVQPGGSLRLSCAASGFTFSTYAMNWVRQAPGKCLEWVGRIRSKANNYATYYADSVKGRFTISRDDSKNTLYLQMNSLRAEDTAVYYCVRHGNFGDSYVSWFAYWGQGTLVTVSS

SEQ ID NO: 210 - protein, 5 aa, Artificial Sequence
  Description of Artificial Sequence Synthetic peptide
  Disulfide Stabilized Anti-CD3-High-Int#1 - [anti-CD3]_ H1.153_L1.64_scFv vhCDR1
TYAMN

SEQ ID NO: 211 - protein, 19 aa, Artificial Sequence
  Description of Artificial Sequence Synthetic peptide
  Disulfide Stabilized Anti-CD3-High-Int#1 - [anti-CD3]_ H1.153_L1.64_scFv vhCDR2
RIRSKANNYATYYADSVKG

SEQ ID NO: 212 - protein, 14 aa, Artificial Sequence
  Description of Artificial Sequence Synthetic peptide
  Disulfide Stabilized Anti-CD3-High-Int#1 - [anti-CD3]_ H1.153_L1.64_scFv vhCDR3
HGNFGDSYVSWFAY

SEQ ID NO: 213 - protein, 109 aa, Artificial Sequence; identical to SEQ ID NO: 203
  Description of Artificial Sequence Synthetic polypeptide
  Disulfide Stabilized Anti-CD3-High-Int#1 - [anti-CD3]_ H1.153_L1.64_scFv Variable Light (vl) Domain

SEQ ID NO: 214 - protein, 14 aa, Artificial Sequence
  Description of Artificial Sequence Synthetic peptide
  Disulfide Stabilized Anti-CD3-High-Int#1 - [anti-CD3]_ H1.153_L1.64_scFv vlCDR1
GSSTGAVTTSNYAN

SEQ ID NO: 215 - protein, 7 aa, Artificial Sequence
  Description of Artificial Sequence Synthetic peptide
  Disulfide Stabilized Anti-CD3-High-Int#1 - [anti-CD3]_ H1.153_L1.64_scFv vlCDR2
GTNKRAP

SEQ ID NO: 216 - protein, 9 aa, Artificial Sequence
  Description of Artificial Sequence Synthetic peptide
  Disulfide Stabilized Anti-CD3-High-Int#1 - [anti-CD3]_ H1.153_L1.64_scFv vlCDR3
ALWYSNHWV

SEQ ID NO: 217 - protein, 20 aa, Artificial Sequence
  Description of Artificial Sequence Synthetic peptide
  Disulfide Stabilized Anti-CD3-High-Int#1 - [anti-CD3]_ H1.153_L1.64_scFv Linker
GKPGSGKPGSGKPGSGKPGS

SEQ ID NO: 218 - protein, 254 aa, Artificial Sequence
  Description of Artificial Sequence Synthetic polypeptide
  Disulfide Stabilized Anti-CD3-High-Int#2 - [anti-CD3]_ H1.154_L1.64_scFv scFv
EVQLVESGGGLVQPGGSLRLSCAASGFTFSTYAMNWVRQAPGKCLEWVGRIRSKYNNYATYYADSVKGRFTISRDDSKNTLYLQMNSLRAEDTAVYYCVRHGNFGDEYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGCGTKLTVL

SEQ ID NO: 219 - protein, 125 aa, Artificial Sequence
  Description of Artificial Sequence Synthetic polypeptide
  Disulfide Stabilized Anti-CD3-High-Int#2 - [anti-CD3]_ H1.154_L1.64_scFv Variable Heavy (vh) Domain
EVQLVESGGGLVQPGGSLRLSCAASGFTFSTYAMNWVRQAPGKCLEWVGRIRSKYNNYATYYADSVKGRFTISRDDSKNTLYLQMNSLRAEDTAVYYCVRHGNFGDEYVSWFAYWGQGTLVTVSS

SEQ ID NO: 220 - protein, 5 aa, Artificial Sequence
  Description of Artificial Sequence Synthetic peptide
  Disulfide Stabilized Anti-CD3-High-Int#2 - [anti-CD3]_ H1.154_L1.64_scFv vhCDR1
TYAMN

SEQ ID NO: 221 - protein, 19 aa, Artificial Sequence
  Description of Artificial Sequence Synthetic peptide
  Disulfide Stabilized Anti-CD3-High-Int#2 - [anti-CD3]_ H1.154_L1.64_scFv vhCDR2
RIRSKYNNYATYYADSVKG

SEQ ID NO: 222 - protein, 14 aa, Artificial Sequence
  Description of Artificial Sequence Synthetic peptide
  Disulfide Stabilized Anti-CD3-High-Int#2 - [anti-CD3]_ H1.154_L1.64_scFv vhCDR3
HGNFGDEYVSWFAY

SEQ ID NO: 223 - protein, 109 aa, Artificial Sequence; identical to SEQ ID NO: 203
  Description of Artificial Sequence Synthetic polypeptide
  Disulfide Stabilized Anti-CD3-High-Int#2 - [anti-CD3]_ H1.154_L1.64_scFv Variable Light (vl) Domain

SEQ ID NO: 224 - protein, 14 aa, Artificial Sequence
  Description of Artificial Sequence Synthetic peptide
  Disulfide Stabilized Anti-CD3-High-Int#2 - [anti-CD3]_ H1.154_L1.64_scFv vlCDR1
GSSTGAVTTSNYAN

SEQ ID NO: 225 - protein, 7 aa, Artificial Sequence
  Description of Artificial Sequence Synthetic peptide
  Disulfide Stabilized Anti-CD3-High-Int#2 - [anti-CD3]_ H1.154_L1.64_scFv vlCDR2
GTNKRAP

SEQ ID NO: 226 - protein, 9 aa, Artificial Sequence
  Description of Artificial Sequence Synthetic peptide
  Disulfide Stabilized Anti-CD3-High-Int#2 - [anti-CD3]_ H1.154_L1.64_scFv vlCDR3
ALWYSNHWV

SEQ ID NO: 227 - protein, 20 aa, Artificial Sequence
  Description of Artificial Sequence Synthetic peptide
  Disulfide Stabilized Anti-CD3-High-Int#2 - [anti-CD3]_ H1.154_L1.64_scFv Linker
GKPGSGKPGSGKPGSGKPGS

SEQ ID NO: 228 - protein, 254 aa, Artificial Sequence
  Description of Artificial Sequence Synthetic polypeptide
  Disulfide Stabilized Anti-CD3-Intermediate - [anti-CD3]_ H1.155_L1.64_scFv scFv
EVQLVESGGGLVQPGGSLRLSCAASGFTFSTYAMNWVRQAPGKCLEWVGRIRSKYNNYATYYADSVKGRFTISRDDSKNTLYLQMNSLRAEDTAVYYCVRHGNFGDSYVSWFDYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGCGTKLTVL

SEQ ID NO: 229 - protein, 125 aa, Artificial Sequence
  Description of Artificial Sequence Synthetic polypeptide
  Disulfide Stabilized Anti-CD3-Intermediate - [anti-CD3]_ H1.155_L1.64_scFv Variable Heavy (vh) Domain
EVQLVESGGGLVQPGGSLRLSCAASGFTFSTYAMNWVRQAPGKCLEWVGRIRSKYNNYATYYADSVKGRFTISRDDSKNTLYLQMNSLRAEDTAVYYCVRHGNFGDSYVSWFDYWGQGTLVTVSS

SEQ ID NO: 230 - protein, 5 aa, Artificial Sequence
  Description of Artificial Sequence Synthetic peptide
  Disulfide Stabilized Anti-CD3-Intermediate - [anti-CD3]_ H1.155_L1.64_scFv vhCDR1
TYAMN

SEQ ID NO: 231 - protein, 19 aa, Artificial Sequence
  Description of Artificial Sequence Synthetic peptide
  Disulfide Stabilized Anti-CD3-Intermediate - [anti-CD3]_ H1.155_L1.64_scFv vhCDR2
RIRSKYNNYATYYADSVKG

SEQ ID NO: 232 - protein, 14 aa, Artificial Sequence
  Description of Artificial Sequence Synthetic peptide
  Disulfide Stabilized Anti-CD3-Intermediate - [anti-CD3]_ H1.155_L1.64_scFv vhCDR3
HGNFGDSYVSWFDY

SEQ ID NO: 233 - protein, 109 aa, Artificial Sequence; identical to SEQ ID NO: 203
  Description of Artificial Sequence Synthetic polypeptide
  Disulfide Stabilized Anti-CD3-Intermediate - [anti-CD3]_ H1.155_L1.64_scFv Variable Light (vl) Domain

SEQ ID NO: 234 - protein, 14 aa, Artificial Sequence
  Description of Artificial Sequence Synthetic peptide
  Disulfide Stabilized Anti-CD3-Intermediate - [anti-CD3]_ H1.155_L1.64_scFv vlCDR1
GSSTGAVTTSNYAN

SEQ ID NO: 235 - protein, 7 aa, Artificial Sequence
  Description of Artificial Sequence Synthetic peptide
  Disulfide Stabilized Anti-CD3-Intermediate - [anti-CD3]_ H1.155_L1.64_scFv vlCDR2
GTNKRAP

SEQ ID NO: 236 - protein, 9 aa, Artificial Sequence
  Description of Artificial Sequence Synthetic peptide
  Disulfide Stabilized Anti-CD3-Intermediate - [anti-CD3]_ H1.155_L1.64_scFv vlCDR3
ALWYSNHWV

SEQ ID NO: 237 - protein, 20 aa, Artificial Sequence
  Description of Artificial Sequence Synthetic peptide
  Disulfide Stabilized Anti-CD3-Intermediate - [anti-CD3]_ H1.155_L1.64_scFv Linker
GKPGSGKPGSGKPGSGKPGS

SEQ ID NO: 238 - protein, 449 aa, Artificial Sequence; identical to SEQ ID NO: 139
  Description of Artificial Sequence Synthetic polypeptide
  XENP29171 [alphaCD123]_H1.109_IgG1_PVA_/S267K_ pI(-)_Isosteric_A_L368D/K370S Heavy Chain

SEQ ID NO: 239 - protein, 714 aa, Artificial Sequence; identical to SEQ ID NO: 184
  Description of Artificial Sequence Synthetic polypeptide
  XENP29171 [alphaCD3]_H1.152_L1.64_scFv(GKPGS) 4_Clambda_(GGGGS)5_CH1-Fc(216)_S364K/E357Q scFv-scCLCH Chain

SEQ ID NO: 240 - protein, 220 aa, Artificial Sequence; identical to SEQ ID NO: 78
  Description of Artificial Sequence Synthetic polypeptide
  XENP29171 [alphaCD123]_L1.57 Light Chain

SEQ ID NO: 241 - protein, 444 aa, Artificial Sequence; identical to SEQ ID NO: 145
  Description of Artificial Sequence Synthetic polypeptide
  XENC140 [alphaPSMA]_H1_IgG1_PVA_/S267K_pI(-)_ Isosteric_A_L368D/K370S Heavy Chain

SEQ ID NO: 242 - protein, 714 aa, Artificial Sequence; identical to SEQ ID NO: 184
  Description of Artificial Sequence Synthetic polypeptide
  XENC141 [alphaCD3]_H1.152_L1.64_scFv(GKPGS) 4_Clambda_(GGGGS)5_CH1-Fc(216)_S364K/E357Q scFv-scCLCH Chain

SEQ ID NO: 243 - protein, 214 aa, Artificial Sequence; identical to SEQ ID NO: 147
  Description of Artificial Sequence Synthetic polypeptide
  XENC142 [alphaPSMA]_L1 Light Chain

SEQ ID NO: 244 - protein, 451 aa, Artificial Sequence; identical to SEQ ID NO: 148
  Description of Artificial Sequence Synthetic polypeptide
  XENCXXX [alphaSSTR2]_H1.143_IgG1_PVA_/ S267K_pI(-)_Isosteric_A_L368D/K370S Heavy Chain

SEQ ID NO: 245 - protein, 714 aa, Artificial Sequence; identical to SEQ ID NO: 184
  Description of Artificial Sequence Synthetic polypeptide
  XENC144 [alphaCD3]_H1.152_L1.64_scFv(GKPGS) 4_Clambda_(GGGGS)5_CH1-Fc(216)_S364K/E357Q scFv-scCLCH Chain

SEQ ID NO: 246 - protein, 219 aa, Artificial Sequence; identical to SEQ ID NO: 150
  Description of Artificial Sequence Synthetic polypeptide
  XENC145 [alphaSSTR2]_L1.30 Light Chain

SEQ ID NO: 247 - protein, 15 aa, Artificial Sequence
  Description of Artificial Sequence Synthetic peptide
GGGGSGGGGSGGGGS

(19) United States
(12) Patent Application Publication
(10) Pub. No.: US 2021/0102002 A1
(43) Pub. Date: Apr. 8, 2021

(54) HETERODIMERIC IgG-LIKE BISPECIFIC ANTIBODIES

(71) Applicant: Xencor, Inc., Monrovia, CA (US)

(72) Inventors: Matthew Bernett, Monrovia, CA (US); John Desjarlais, Pasadena, CA (US); Eric Chang, Walnut, CA (US)

(21) Appl. No.: 16/987,250

(22) Filed: Aug. 6, 2020

Related U.S. Application Data
(60) Provisional application No. 62/883,542, filed on Aug. 6, 2019.

(51) Int. Cl. C07K 16/46

(52) U.S. Cl. CPC C07K 16/468; C07K 2317/622; C07K 2317/55; C07K 2317/31

(57) ABSTRACT

Novel Heterodimeric IgG-Like Bispecific Antibodies (HILBAs) are provided herein. Such bispecific antibodies are similar to IgG in structure and, thus, do not exhibit a limited half-life like previous antibody fragment-based bispecifics. Moreover, such heterodimeric IgG-like bispecific antibodies advantageously do not entail complicated generation methods such as Fab arm exchange and common light chain methods. Unlike canonical IgG antibodies, the HILBAs described herein include at least one monomer that is from N- to C-terminus: a VH-scFv linker-VL-CL-domain linker-CH1-hinge-CH2-CH3.

### PRIORITY CLAIM

This application claims priority to U.S. Provisional Patent Application Nos. 62/883,542, filed Aug. 6, 2019, the contents of which are each hereby incorporated by reference in their entirety.

### SEQUENCE LISTING

The instant application contains a Sequence Listing which has been submitted electronically in ASCII format and is hereby incorporated by reference in its entirety. Said ASCII copy, created on Oct. 21, 2020, is named 067461-5231-US_SL.txt and is 422,672 bytes in size.

### BACKGROUND

Antibody-based therapeutics have revolutionized the treatment of a variety of diseases, including cancer and autoimmune/inflammatory disorders. Numerous avenues have been explored to introduce novel mechanisms of actions and/or improved efficacy to this class of drugs. One such avenue is the engineering of additional and novel antigen binding sites so that a single antibody-like molecule co-engages two different antigens. Such antibody formats are often referred to as bispecific antibodies or bispecifics.

A number of alternative antibody formats have been explored for bispecific targeting. For example, antibody fragments of many different forms have been generated, including BiTE (Goebeler et al. (2016) Journal Clinical Oncology, 34(10):1104-1111, herein incorporated by reference), DART (Root et al. (2016) Antibodies, 5(1), 6; Tsai et al. (2016) Molecular Therapy Oncolytics, 3, 15024, all of which are herein incorporated by reference), Nanobody (Bannas et al. (2017) Front Immunol, 8: 1603, herein incorporated by reference), and TandAb (Reusch et al. (2014) mAbs, 6:3, 727-738; Reusch et al. (2015) mAbs, 7:3, 584-604, all of which are herein incorporated by reference) formats which have advanced into clinical testing. However, these antibody fragments clear rapidly in vivo as they lack the constant region of intact IgG that provides binding to various Fc receptors and ligands that maintain long half-life in serum (i.e., the neonatal Fc receptor, FcRn). To address such shortcomings of fragment-based bispecifics, formats having Fc regions have been generated, such as the HLE BiTE format (Lorenczewski et al. (2017) Blood, 130:2815; PCT Publication No. WO 2017/134140, all of which are herein incorporated by reference) and the 1+1 Fab-scFv-Fc format (referred to as the Triple F format in PCT Publication No. WO 2014/110601, herein incorporated by reference). Nonetheless, these formats differ structurally from the natural IgG format, and may fall short of the evolutionarily optimized pharmacokinetic properties and immunogenic profile inherent to the natural IgG structure.

Accordingly, bispecific antibodies with IgG-like structures have been generated using approaches such as Fab-arm exchange (Labrijn et al. (2013) PNAS 110(13):5145-5150, herein incorporated by reference) and common light chain (Nardis et al. (2017) JBC, herein incorporated by reference). However, Fab-arm exchange involves complicated production that requires chemical reduction of two antibody species followed by rejoining of appropriate heterodimeric pairs, while the common light chain approach requires extensive screening to identity variable regions that can utilize a common light chain.

Thus, there remains a need for additional alternative bispecific antibody formats.

### BRIEF SUMMARY

Provided herein are novel Heterodimeric IgG-Like Bispecific Antibodies (HILBAs). Such bispecific antibodies are similar to IgG in structure and, thus, do not exhibit a limited half-life like previous antibody fragment-based bispecifics. Moreover, such heterodimeric IgG-like bispecific antibodies advantageously do not entail complicated generation methods such as Fab arm exchange and common light chain methods.

In one aspect, provided herein is a HILBA that is of the (scFv-scCLCH)₁ format. Such antibodies include a) a first monomer that includes a VH1-CH1-hinge-CH2-CH3, wherein VH1 is a first variable heavy domain and CH2-CH3 is a first Fc domain; b) a second monomer that includes VL1-CL, wherein VL1 is a first variable light domain; and c) a third monomer that includes VH2-scFv linker-VL2-CL-domain linker-CH1-hinge-CH2-CH3, wherein VH2 is a second variable domain, VL2 is a second variable light domain, and CH2-CH3 is a second Fc domain. In such (scFv-scCLCH)₁ format HILBAs, the VH1 and VL1 form a first antigen binding domain, and VH2 and VL2 form a second antigen binding domain that binds a different antigen than the first antigen binding domain.

In some embodiments, the (scFv-scCLCH)₁ format antibodies include dsHC variants wherein the VH2 includes a first engineered cysteine residue and the CH1 of the third monomer includes a second engineered cysteine residue, and wherein the first engineered cysteine residue and the second engineered cysteine residue form a disulfide bond. In some embodiments, the first engineered cysteine residue is a S112C or S113C amino acid substitution in VH2, wherein numbering is according to Kabat numbering. In certain embodiments, the second engineered cysteine residue is an A118C amino acid substitution in CH1 of the third monomer, wherein numbering is according to EU numbering.

In some embodiments, the (scFv-scCLCH)₁ format antibodies include dsscFv variants wherein the VH2 includes a first engineered cysteine residue, VL2 includes a second engineered cysteine residue and wherein the first engineered cysteine residue and the second engineered cysteine residue form a disulfide bond. In exemplary embodiments, the first engineered cysteine residue is a G44C amino acid substitution in VH2, wherein numbering is according to Kabat numbering. In certain embodiments, the second engineered cysteine residue is a G100C amino acid substitution in VL2, wherein numbering is according to Kabat numbering.

In some embodiments of the (scFv-scCLCH)₁ format antibodies, the VH2 includes a first engineered cysteine residue and a second engineered cysteine residue, the CH1 of the third monomer includes a third engineered cysteine residue, and the VL2 includes a fourth engineered cysteine residue. In such embodiments, the first engineered cysteine residue and the third engineered cysteine residue form a first disulfide bond, and the second engineered cysteine residue and the fourth engineered cysteine residue form a second disulfide bond.

In certain embodiments of the (scFv-scCLCH)₁ format antibodies, the first Fc domain and the second Fc domain are each variant Fc domains that include a skew variant set, where the skew variant set is one of the following: S267K/L368D/K370S:S267K/S364K/E357Q; S364K/E357Q:L368D/K370S; L368D/K370S:S364K; L368E/K370S:S364K; T411E/K360E/Q362E:D401K; L368D/K370S:S364K/E357L and K370S:S364K/E357Q, according to EU numbering. In an exemplary embodiment, the skew variant set is S364K/E357Q:L368D/K370S.

In one embodiment of the (scFv-scCLCH)₁ format antibodies, the first Fc domain is a variant Fc domain that includes FcKO variants E233P/L234V/L235A/G236 del/S267K. In certain embodiments, the first Fc domain is a variant Fc domain that includes pI variants Q295E/N384D/Q418E/N421D. In certain embodiments, the second Fc domain is a variant Fc domain that includes pI variants Q295E/N384D/Q418E/N421D.

In an exemplary embodiment of the (scFv-scCLCH)₁ format antibody, the first variant Fc domain includes skew variants L368D/K370S and the second variant Fc domain includes skew variants S364K/E357Q, the first variant Fc domain and second variant Fc domain each include FcKO variants E233P/L234V/L235A/G236del/S267K, and the first variant Fc domain or second variant Fc domain includes pI variants Q295E/N384D/Q418E/N421D. In some embodiments, the VH2 includes a first engineered cysteine residue and the CH1 of the third monomer includes a second engineered cysteine residue, wherein the first engineered cysteine residue and the second engineered cysteine residue form a disulfide bond. In certain embodiments, the VH2 includes a first engineered cysteine residue, and VL2 includes a second engineered cysteine residue, wherein the first engineered cysteine residue and the second engineered cysteine residue form a disulfide bond. In an exemplary embodiment, VH2 includes a first engineered cysteine residue and a second engineered cysteine residue, the CH1 of the third monomer includes a third engineered cysteine residue, and the VL2 includes a fourth engineered cysteine residue, wherein the first engineered cysteine residue and the third engineered cysteine residue form a first disulfide bond, and the second engineered cysteine residue and the fourth engineered cysteine residue form a second disulfide bond.

In a second aspect, provided herein are HILBAs having the (scFv-scCLCH)₂ format. Such antibodies include: a) a first monomer that includes a VH1-scFv linker-VL1-CL-domain linker-CH1-hinge-CH2-CH3, wherein VH1 is a first variable domain, VL1 is a first variable light domain, and CH2-CH3 is a first Fc domain; and b) a second monomer that includes a VH2-scFv linker-VL2-CL-domain linker-CH1-hinge-CH2-CH3, wherein VH2 is a second variable domain, VL2 is a second variable light domain, and CH2-CH3 is a second Fc domain. In such a format, VH1 and VL1 form a first antigen binding domain, and VH2 and VL2 form a second antigen binding domain that binds a different antigen than the first antigen binding domain.

In some embodiments of the (scFv-scCLCH)₂ format, VH1 includes a first engineered cysteine residue and CH1 of the first monomer includes a second engineered cysteine residue, VH2 includes a third engineered cysteine residue and CH1 of the second monomer includes a fourth engineered cysteine residue, wherein the first engineered cysteine residue and the second engineered cysteine residue form a first disulfide bond; and the third engineered cysteine residue and the fourth engineered cysteine residue form a second disulfide bond. In some embodiments, the first and third engineered cysteine residues are each a S112C or S113C amino acid substitution, wherein numbering is according to Kabat numbering. In an exemplary embodiment, the second and fourth engineered cysteine residues are each an A118C amino acid substitution, wherein numbering is according to EU numbering.

In some embodiments of the (scFv-scCLCH)₂ format, VH1 includes a first engineered cysteine residue, VL1 includes a second engineered cysteine residue, VH2 includes a third engineered cysteine residue, and VL2 includes a fourth engineered cysteine residue. In such embodiments, the first engineered cysteine residue and the second engineered cysteine residue form a first disulfide bond and the third cysteine residue and fourth engineered cysteine residue from a second disulfide bond. In some embodiments, the first and third engineered cysteine residues are each a G44C amino acid substitution, wherein numbering is according to Kabat numbering. In some embodiments, the second and fourth engineered cysteine residues are each a G100C amino acid substitution, wherein numbering is according to Kabat numbering.

In some embodiments of the (scFv-scCLCH)₂ format, the VH1 includes a first engineered cysteine residue and a second engineered cysteine residue, the CH1 of the first monomer includes a third engineered cysteine residue, the VL1 includes a fourth engineered cysteine residue, the VH2 includes a fifth engineered cysteine residue and a sixth engineered cysteine residue, the CH1 of the second monomer includes a seventh engineered cysteine residue, and the VL2 includes an eighth engineered cysteine residue. In such embodiments, the first engineered cysteine residue and the third engineered cysteine residue form a first disulfide bond, the second engineered cysteine residue and the fourth engineered cysteine residue form a second disulfide bond, the fifth engineered cysteine residue and the seventh engineered cysteine residue from a third disulfide bond, and the sixth engineered cysteine residue and eighth engineered cysteine residue from a fourth disulfide bond.

In some embodiments of the (scFv-scCLCH)₂ format, the first Fc domain and the second Fc domain are each variant Fc domains that include a skew variant set selected from the following: S267K/L368D/K370S:S267K/S364K/E357Q; S364K/E357Q:L368D/K370S; L368D/K370S:S364K; L368E/K370S:S364K; T411E/K360E/Q362E:D401K; L368D/K370S:S364K/E357L and K370S:S364K/E357Q, according to EU numbering. In an exemplary embodiment, the skew variant set is S364K/E357Q:L368D/K370S. In certain embodiments, the first Fc domain is a variant Fc domain that includes FcKO variants E233P/L234V/L235A/G236del/S267K. In certain embodiments, the first Fc domain is a variant Fc domain that includes pI variants Q295E/N384D/Q418E/N421D. In certain embodiments, the second Fc domain is a variant Fc domain that includes pI variants Q295E/N384D/Q418E/N421D.

In exemplary embodiments of the (scFv-scCLCH)₂ format, the first variant Fc domain includes skew variants L368D/K370S and the second variant Fc domain includes skew variants S364K/E357Q, the first variant Fc domain and second variant Fc domain each include FcKO variants E233P/L234V/L235A/G236del/S267K, and the first or second variant Fc domain includes pI variants Q295E/N384D/Q418E/N421D.

In some embodiments, VH1 includes a first engineered cysteine residue and CH1 of the first monomer includes a second engineered cysteine residue, VH2 includes a third engineered cysteine residue and CH1 of the second monomer includes a fourth engineered cysteine residue, wherein the first engineered cysteine residue and the second engineered cysteine residue form a first disulfide bond, and the third engineered cysteine residue and the fourth engineered cysteine residue form a second disulfide bond.

In certain embodiments, VH1 includes a first engineered cysteine residue, VL1 includes a second engineered cysteine residue, VH2 includes a third engineered cysteine residue, and VL2 includes a fourth engineered cysteine residue, wherein the first engineered cysteine residue and the second engineered cysteine residue form a first disulfide bond and the third cysteine residue and fourth engineered cysteine residue from a second disulfide bond.

In an exemplary embodiment, the VH1 includes a first engineered cysteine residue and a second engineered cysteine residue, the CH1 of the first monomer includes a third engineered cysteine residue, the VL1 includes a fourth engineered cysteine residue, the VH2 includes a fifth engineered cysteine residue and a sixth engineered cysteine residue, the CH1 of the second monomer includes a seventh engineered cysteine residue, and the VL2 includes an eighth engineered cysteine residue, wherein the first engineered cysteine residue and the third engineered cysteine residue form a first disulfide bond, the second engineered cysteine residue and the fourth engineered cysteine residue form a second disulfide bond, the fifth engineered cysteine residue and the seventh engineered cysteine residue from a third disulfide bond, and the sixth engineered cysteine residue and eighth engineered cysteine residue from a fourth disulfide bond.

In another aspect, provided herein is a heterodimeric protein that includes: a) a first monomer that includes a CH2-CH3, wherein CH2-CH3 is a first Fc domain; and b) a second monomer that includes a VH-scFv linker-VL-CL-domain linker-CH1-hinge-CH2-CH3, wherein VH is a variable domain, VL is a variable light domain, and CH2-CH3 is a second Fc domain, and wherein VH and VL form an antigen binding domain.

In some embodiments, VH includes a first engineered cysteine residue and the CH1 of the second monomer includes a second engineered cysteine residue, wherein the first engineered cysteine residue and the second engineered cysteine residue form a disulfide bond. In some embodiments, the first engineered cysteine residue is a S112C or S113C amino acid substitution in VH, wherein numbering is according to Kabat numbering. In certain embodiments, the second engineered cysteine residue is a A118C amino acid substitution in CH1, wherein numbering is according to EU numbering.

In some embodiments, VH includes a first engineered cysteine residue, VL comprises a second engineered cysteine residue and wherein the first engineered cysteine residue and the second engineered cysteine residue form a disulfide bond. In exemplary embodiments, the first engineered cysteine residue is a G44C amino acid substitution in VH, wherein numbering is according to Kabat numbering. In several embodiments, the second engineered cysteine residue is a G100C amino acid substitution in VL, wherein numbering is according to Kabat numbering.

In exemplary embodiments, the VH includes a first engineered cysteine residue and a second engineered cysteine residue, CH1 includes a third engineered cysteine residue, and the VL includes a fourth engineered cysteine residue. In such embodiments, the first engineered cysteine residue and the third engineered cysteine residue form a first disulfide bond, and the second engineered cysteine residue and the fourth engineered cysteine residue form a second disulfide bond.

In some embodiments, the first Fc domain and the second Fc domain are each variant Fc domains that include a skew variant set selected from the following: S267K/L368D/K370S:S267K/S364K/E357Q; S364K/E357Q:L368D/K370S; L368D/K370S:S364K; L368E/K370S:S364K; T411E/K360E/Q362E:D401K; L368D/K370S:S364K/E357L and K370S:S364K/E357Q, according to EU numbering. In exemplary embodiments, the skew variant set is S364K/E357Q:L368D/K370S. In some embodiments, the first Fc domain and second Fc domain are variant Fc domains that each include FcKO variants E233P/L234V/L235A/G236del/S267K. In exemplary embodiments, the first Fc domain or second Fc domain is a variant Fc domain that includes pI variants Q295E/N384D/Q418E/N421D.

In yet another aspect, provided herein is a heterodimeric protein that includes: a) a first monomer that include a CH2-CH3, wherein CH2-CH3 is a first variant Fc domain; and b) a second monomer that includes a VH-scFv linker-VL-CL-domain linker-CH1-hinge-CH2-CH3, wherein VH is a variable domain, VL is a variable light domain, and CH2-CH3 is a second variant Fc domain, wherein VH and VL form an antigen binding domain, wherein the first variant Fc domain includes skew variants L368D/K370S and the second variant Fc domain includes skew variants S364K/E357Q, wherein the first variant Fc domain and second variant Fc domain each include FcKO variants E233P/L234V/L235A/G236del/S267K, and wherein the first variant Fc domain or second variant Fc domain includes pI variants Q295E/N384D/Q418E/N421D.

In some embodiments, VH includes a first engineered cysteine residue and the CH1 of the second monomer includes a second engineered cysteine residue. In such embodiments, the first engineered cysteine residue and the second engineered cysteine residue form a disulfide bond. In some embodiments, the VH includes a first engineered cysteine residue, and the VL includes a second engineered cysteine residue. In such embodiments, the first engineered cysteine residue and the second engineered cysteine residue form a disulfide bond.

In some embodiments, the VH includes a first engineered cysteine residue and a second engineered cysteine residue, the CH1 of the second monomer includes a third engineered cysteine residue, and the VL comprises a fourth engineered cysteine residue. In such embodiments, the first engineered cysteine residue and the third engineered cysteine residue form a first disulfide bond, and the second engineered cysteine residue and the fourth engineered cysteine residue form a second disulfide bond.

Also provided herein are nucleic acid compositions encoding the HILBAs and heterodimeric proteins provided herein, expression vectors that include such nucleic acids, host cells that include such expression vectors, as well as methods of making the subject HILBAs and heterodimeric proteins.

### BRIEF DESCRIPTION OF THE DRAWINGS

FIGS. 1A-1E depict useful pairs of Fc heterodimerization variant sets (including skew and pI variants). There are variants for which there are no corresponding “monomer 2” variants; these are pI variants which can be used alone on either monomer.

FIG. 2 depicts a list of isosteric variant antibody constant regions and their respective substitutions. pI_(−) indicates lower pI variants, while pI_(+) indicates higher pI variants. These can be optionally and independently combined with other heterodimerization variants of the inventions (and other variant types as well, as outlined herein.) FIG. 3 depicts a list of isosteric variant antibody constant regions and their respective substitutions. pI_(−) indicates lower pI variants, while pI_(+) indicates higher pI variants. These variants can be optionally and independently combined with other variants, including heterodimerization variants, outlined herein.

FIG. 3 depicts useful ablation variants that ablate FcγR binding (sometimes referred to as “knock outs” or “KO” variants). Generally, ablation variants are found on both monomers, although in some cases they may be on only one monomer.

FIG. 4 depicts a particularly useful embodiment of “non-Fv” components of the invention.

FIG. 5 depicts a number of charged scFv linkers that find use in increasing or decreasing the pI of heterodimeric antibodies that utilize one or more scFv as a component. The (+H) positive linker finds particular use herein. A single prior art scFv linker with single charge is referenced as “Whitlow”, from Whitlow et al., Protein Engineering 6(8):989-995 (1993). It should be noted that this linker was used for reducing aggregation and enhancing proteolytic stability in scFvs.

FIG. 6 depicts a number of exemplary domain linkers. In some embodiments, these linkers find use linking the C-terminus of a constant light domain to the N-terminus of a constant heavy domain.

FIGS. 7A-7D depict the sequences of several useful HILBA format heavy constant domain backbones based on human IgG1, without the Fv sequences (e.g. the scFv or the Fab). Backbone 1 is based on human IgG1 (356E/358M allotype), and includes the L368D/K370S skew variants and the N208D/Q295E/N384D/Q418E/N421D pI variants on a first heavy chain constant domain, the S364K/E357Q skew variants on a second heavy chain constant domain, and the E233P/L234V/L235A/G236del/S267K ablation variants on both heavy chain constant domains. Backbone 2 is based on human IgG1 (356E/358M allotype), and includes the L368D/K370S skew variants and the N208D/Q295E/N384D/Q418E/N421D pI variants on a first heavy chain constant domain, the S364K skew variant on a second heavy chain constant domain, and the E233P/L234V/L235A/G236del/S267K ablation variants on both heavy chain constant domains. Backbone 3 is based on human IgG1 (356E/358M allotype), and includes the L368E/K370S skew variants and the N208D/Q295E/N384D/Q418E/N421D pI variants on a first heavy chain constant domain, the S364K skew variant on a second heavy chain constant domain, and the E233P/L234V/L235A/G236del/S267K ablation variants on both heavy chain constant domains. Backbone 4 is based on human IgG1 (356E/358M allotype), and includes the K360E/Q362E/T411E skew variants and the N208D/Q295E/N384D/Q418E/N421D pI variants on a first heavy chain constant domain, the D401K skew variant on a second heavy chain constant domain, and the E233P/L234V/L235A/G236del/S267K ablation variants on both heavy chain constant domains. Backbone 5 is based on human IgG1 (356D/358L allotype), and includes the L368D/K370S skew variants and the N208D/Q295E/N384D/Q418E/N421D pI variants on a first heavy chain constant domain, the S364K/E357Q skew variants on a second heavy chain constant domain, and the E233P/L234V/L235A/G236del/S267K ablation variants on both heavy chain constant domains. Backbone 6 is based on human IgG1 (356E/358M allotype), and includes the L368D/K370S skew variants and the N208D/Q295E/N384D/Q418E/N421D pI variants on a first heavy chain constant domain, the S364K/E357Q skew variants on a second heavy chain constant domain, and the E233P/L234V/L235A/G236del/S267K ablation variants and N297A variant that removes glycosylation on both heavy chain constant domains. Backbone 7 is based on human IgG1 (356E/358M allotype), and includes the L368D/K370S skew variants and the N208D/Q295E/N384D/Q418E/N421D pI variants on a first heavy chain constant domain, the S364K/E357Q skew variants on a second heavy chain constant domain, and the E233P/L234V/L235A/G236del/S267K ablation variants and N297S variant that removes glycosylation on both heavy chain constant domains. Backbone 8 is based on human IgG4, and includes the L368D/K370S skew variants and the N208D/Q295E/N384D/Q418E/N421D pI variants on a first heavy chain constant domain, the S364K/E357Q skew variants on a second heavy chain constant domain, and the S228P (according to EU numbering, S241P in Kabat) variant that ablates Fab arm exchange (as is known in the art) on both heavy chain constant domains. Backbone 9 is based on human IgG2, and includes the L368D/K370S skew variants and the N208D/Q295E/N384D/Q418E/N421D pI variants on a first heavy chain constant domain, and the S364K/E357Q skew variants on a second heavy chain constant domain. Backbone 10 is based on human IgG2, and includes the L368D/K370S skew variants and the N208D/Q295E/N384D/Q418E/N421D pI variants on a first heavy chain constant domain, the S364K/E357Q skew variants on a second heavy chain constant domain, and the S267K ablation variant on both heavy chain constant domains. Backbone 11 is based on human IgG1 (356E/358M allotype), and includes the L368D/K370S skew variants and the N208D/Q295E/N384D/Q418E/N421D pI variants on a first heavy chain constant domain, the S364K/E357Q skew variants on a second heavy chain constant domain, and the E233P/L234V/L235A/G236del/S267K ablation variants and M428L/N434S Xtend variants on both heavy chain constant domains. Backbone 12 is based on human IgG1 (356E/358M allotype, and includes the L368D/K370S skew variants on a first heavy chain constant domain, the S364K/E357Q skew variants and the Q196K/I199T/P217R/P229R/N276K pI variants on a second heavy chain constant domain, and the E233P/L234V/L235A/G236del/S267K ablation variants on both heavy chain constant domains.

As will be appreciated by those in the art, these backbone sequences can be used with any Fab and scFv pair or with any scFv pair. Further as will be appreciated by those in the art, for each of these backbone pairs, the first and/or second constant heavy domain monomer can include an engineered cysteine in the N-terminus including, but not limited to, A118C (numbering as in EU or A114C based on Kabat numbering) so as to form a disulfide pair with an scFv in the context of, for example, the (scFv-scCLCH)₁(dsHC), (scFv-scCLCH)₁ (dsHC/dsscFv), (scFv-scCLCH)₂(dsHC), or (scFv-scCLCH)₂(dsHC/dsscFv) formats.

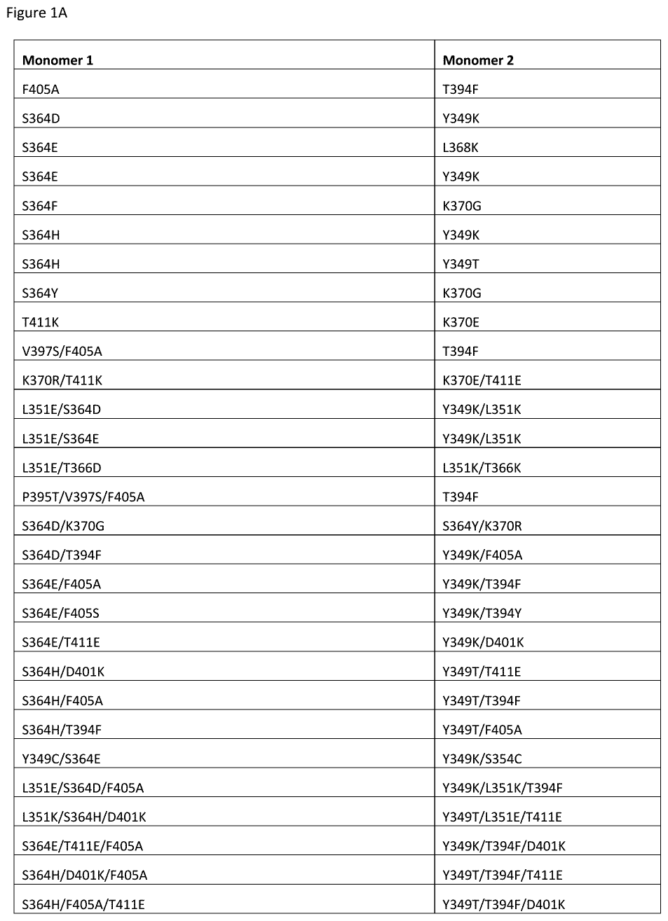

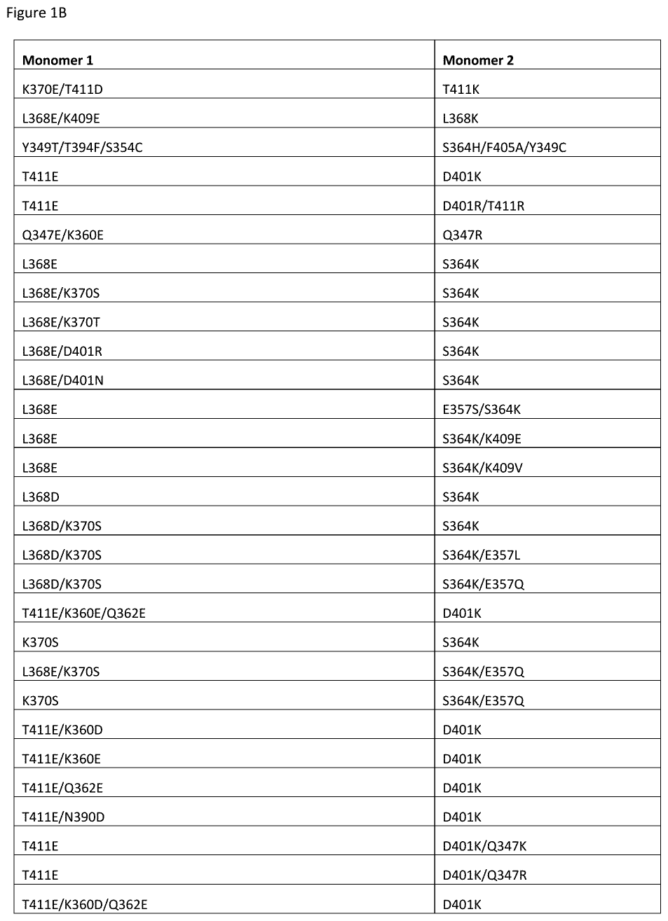

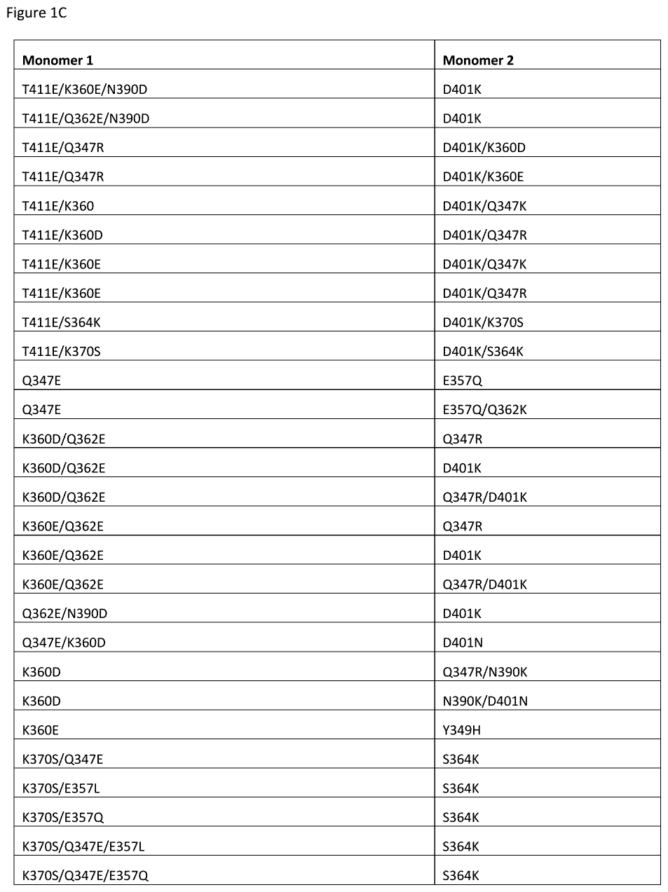

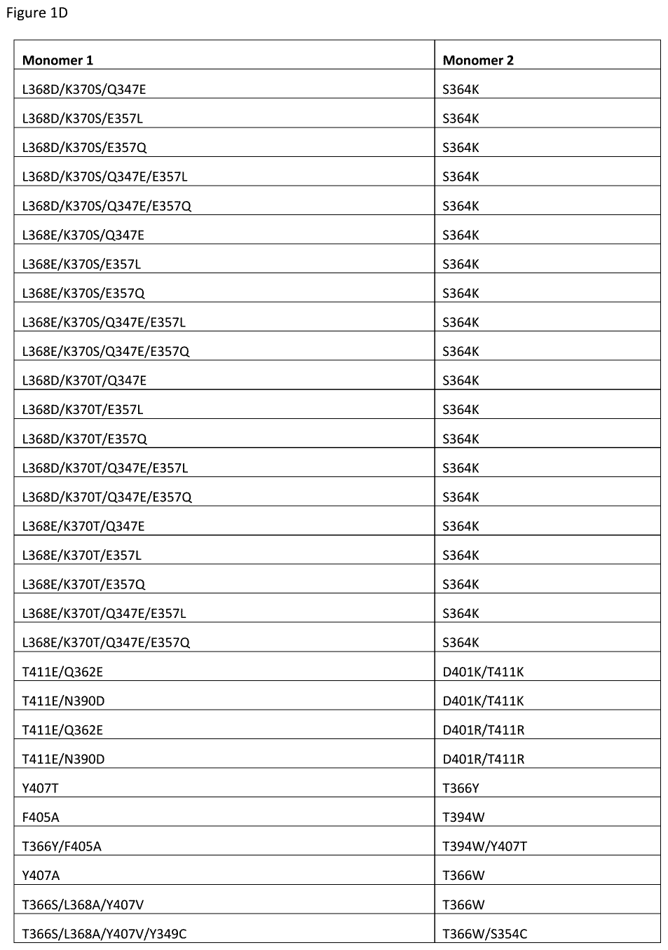

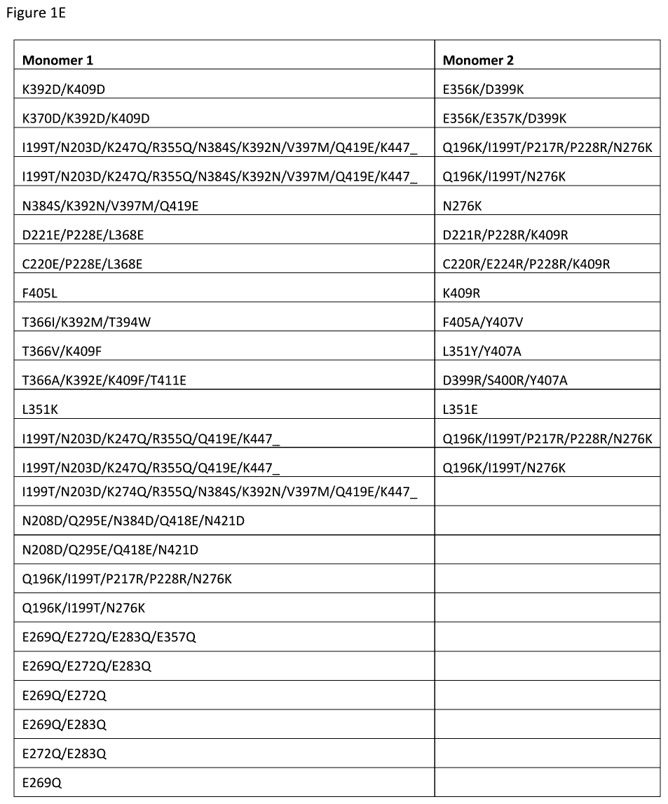

Included within each of these backbones are sequences that are 90, 95, 98 and 99% identical (as defined herein) to the recited sequences, and/or contain from 1, 2, 3, 4, 5, 6, 7, 8, 9 or 10 additional amino acid modifications (as compared to the “parent” of the Figure, which, as will be appreciated by those in the art, already contain a number of amino acid modifications as compared to the parental human IgG1 (or IgG2 or IgG4, depending on the backbone)). That is, the recited backbones may contain additional or alternative amino acid modifications (generally amino acid substitutions) in addition or as an alternative to the skew, pI and ablation variants contained within the backbones of this Figure. Suitable additional or alternative variants include, but are not limited to, those depicted in FIGS. 1-3.

FIG. 8 depicts the sequences of several useful HILBA format constant light domain backbones based on human IgG1, without the Fv sequences (e.g. the scFv or the Fab). Included herein are constant light backbone sequences that are 90, 95, 98 and 99% identical (as defined herein) to the recited sequences, and/or contain from 1, 2, 3, 4, 5, 6, 7, 8, 9 or 10 additional amino acid modifications.

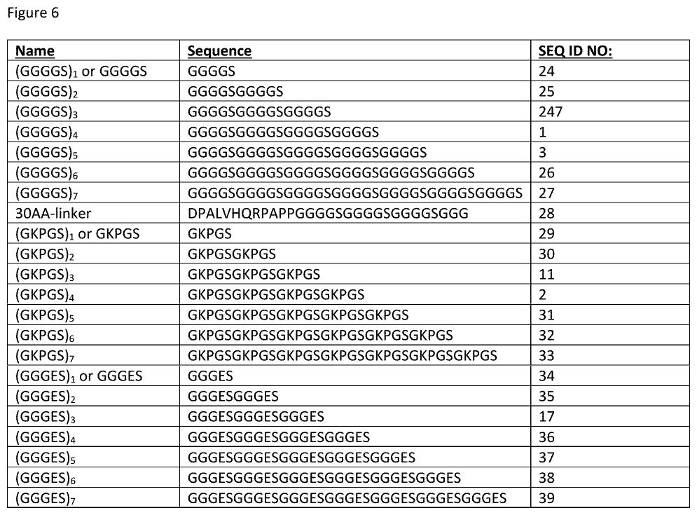

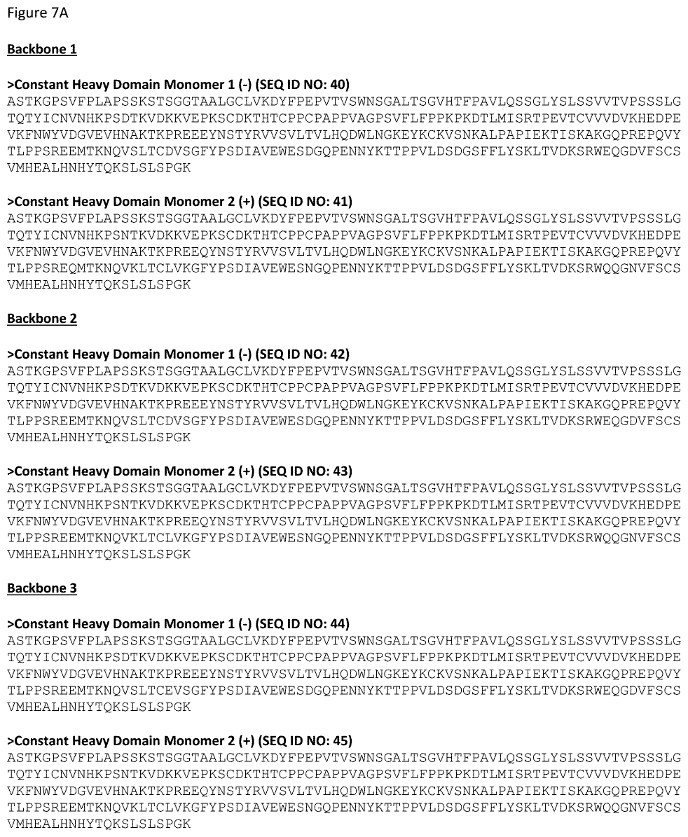

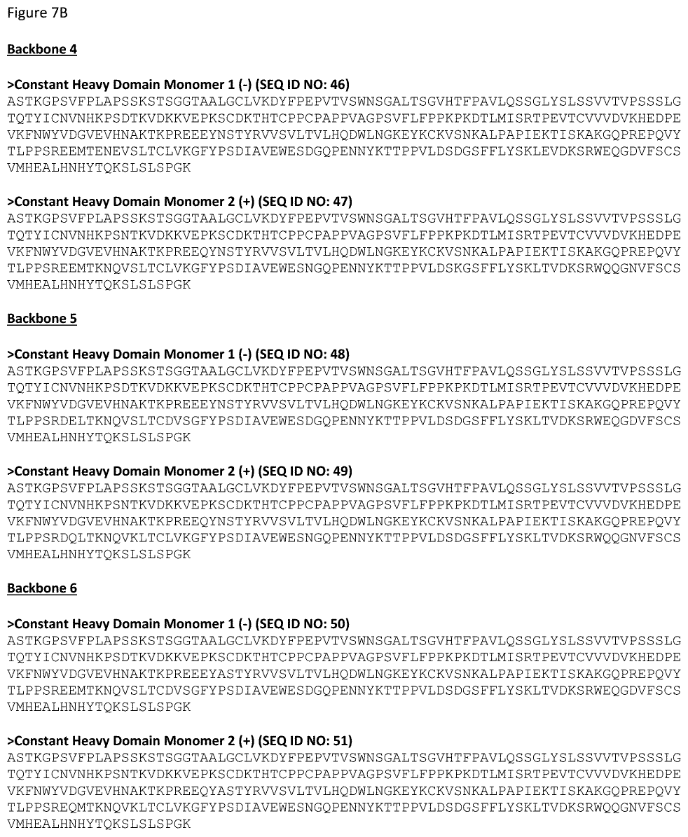

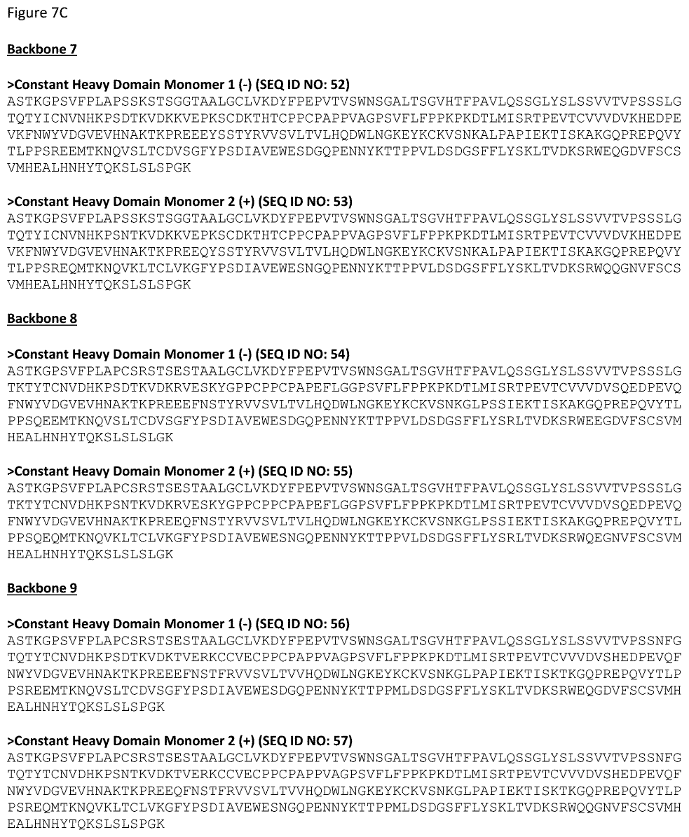

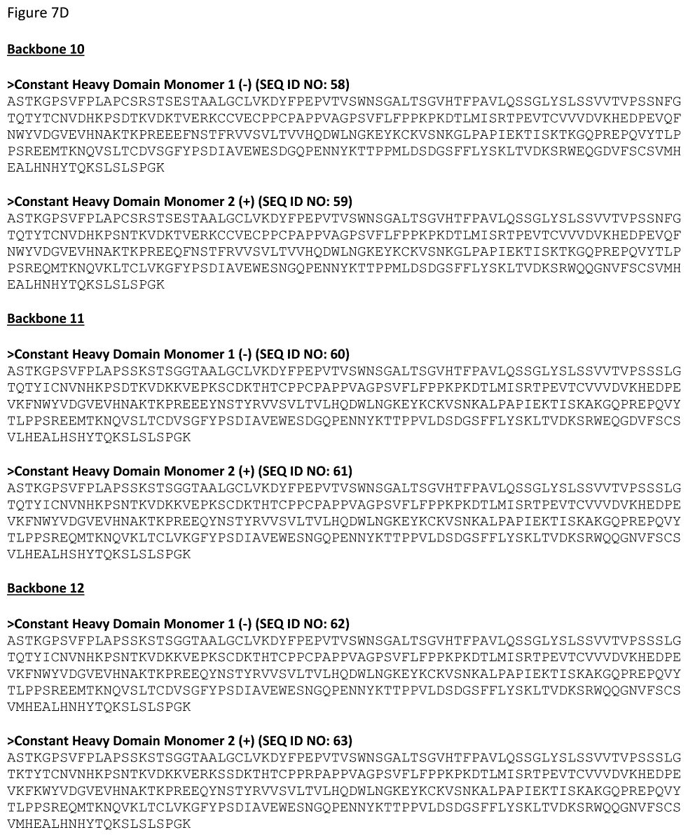

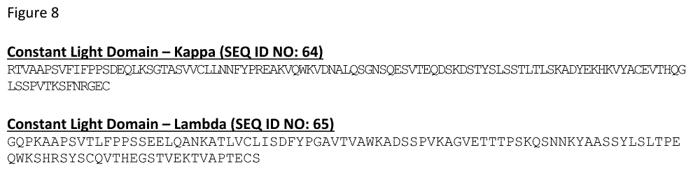

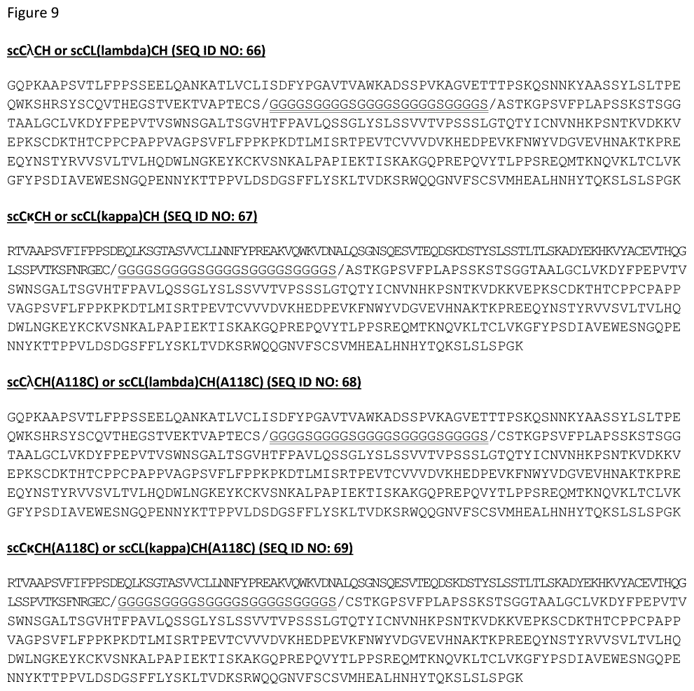

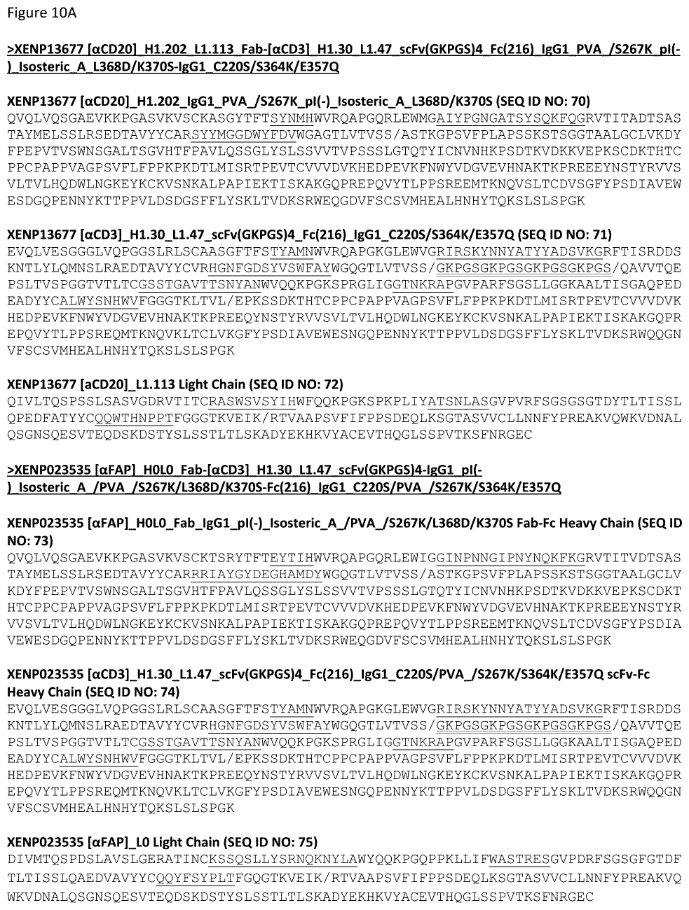

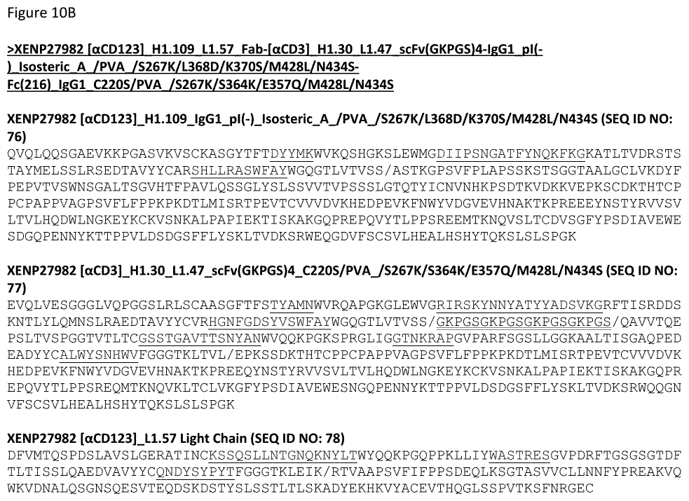

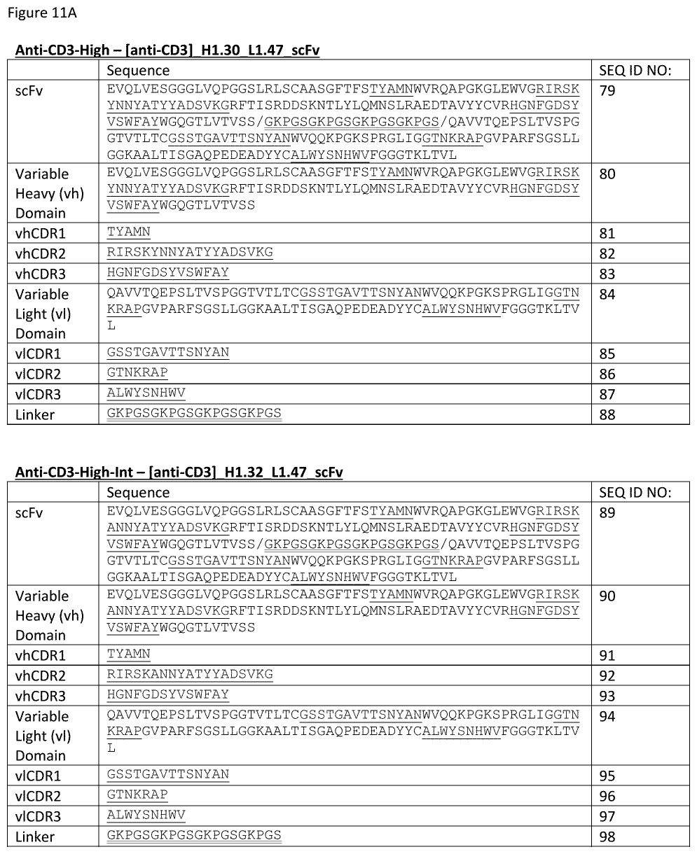

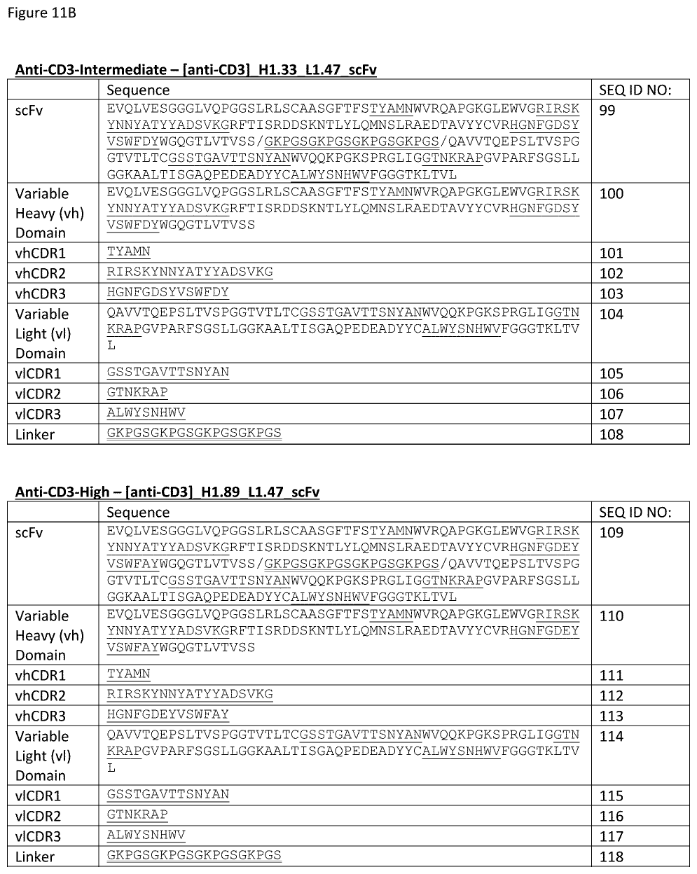

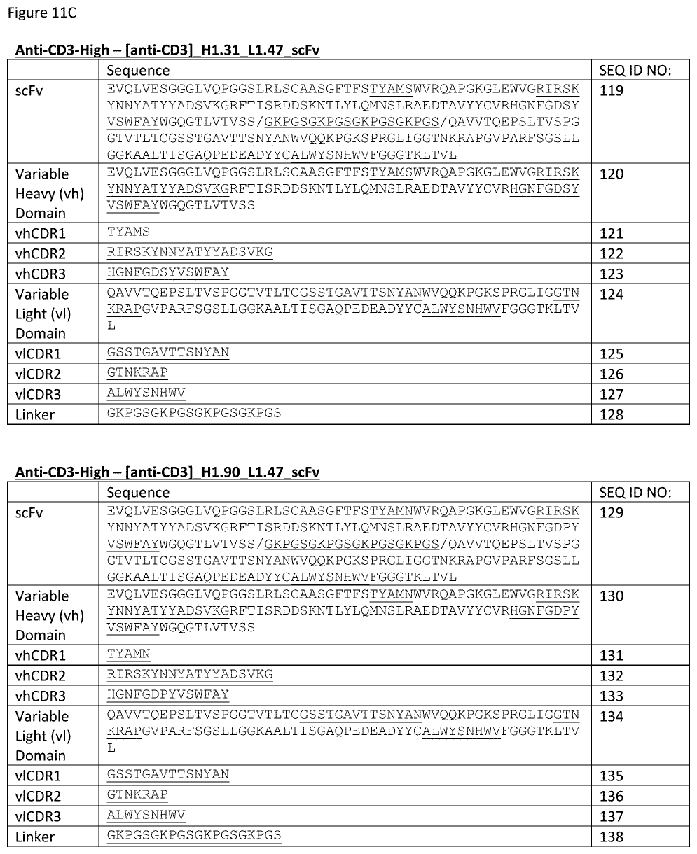

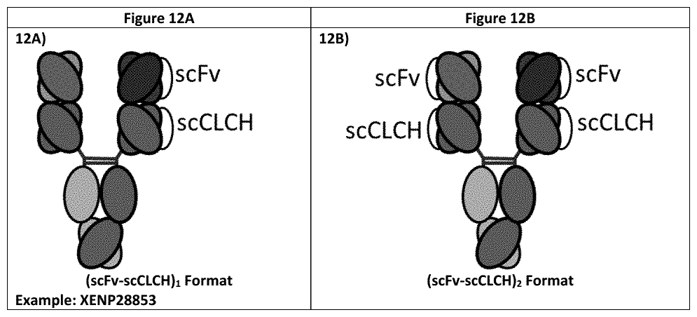

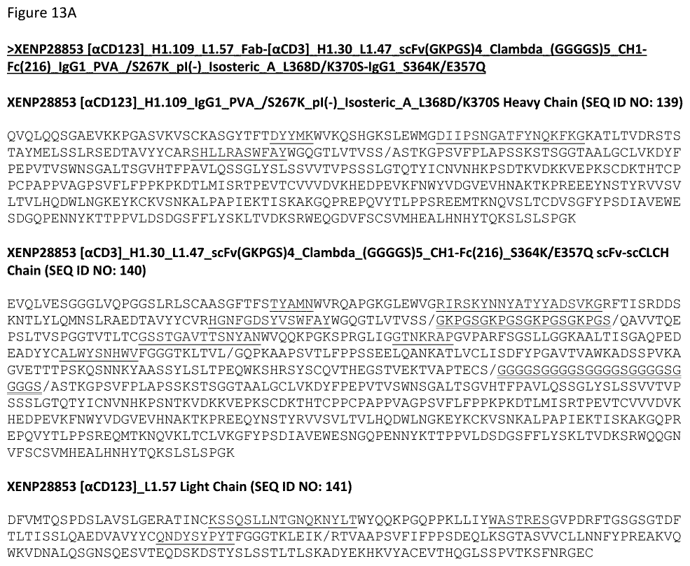

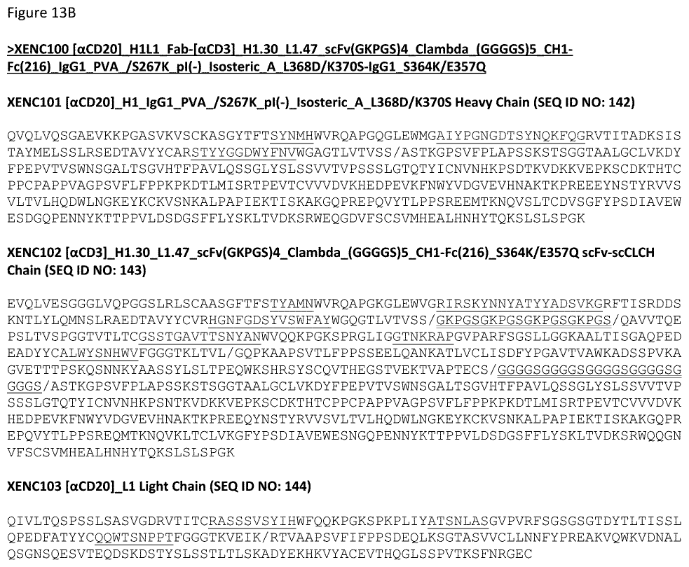

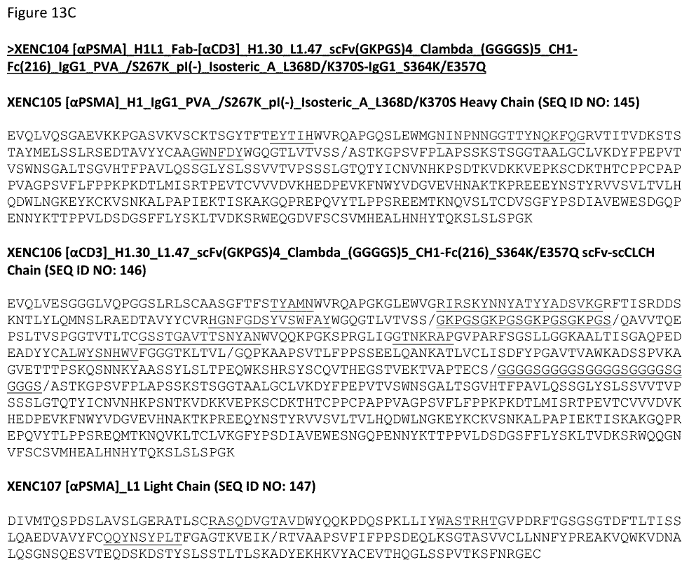

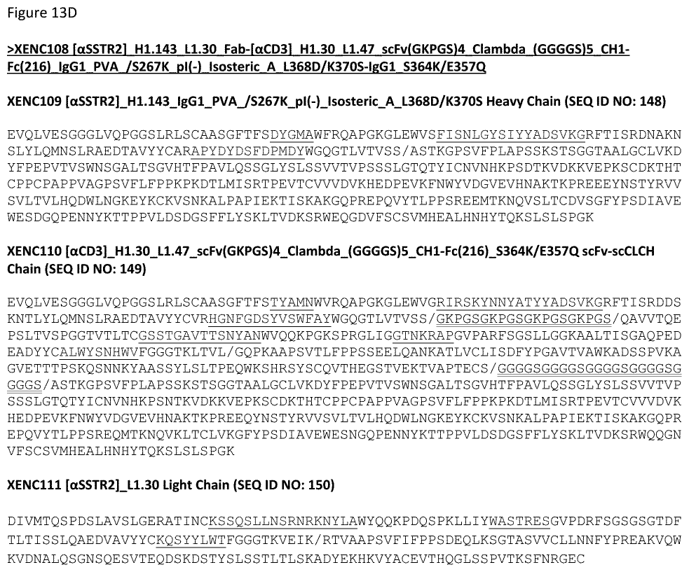

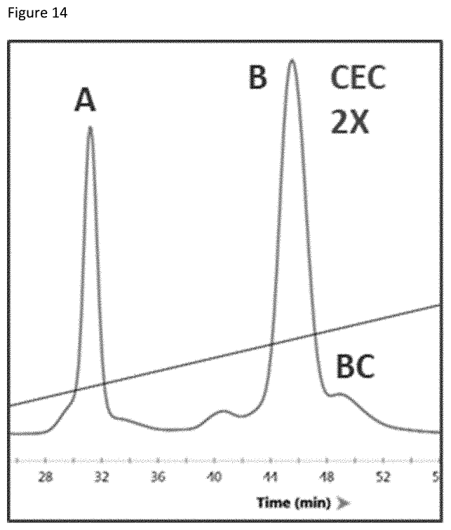

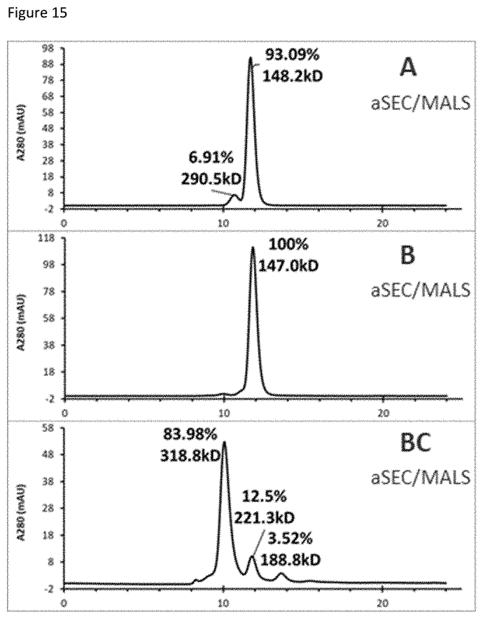

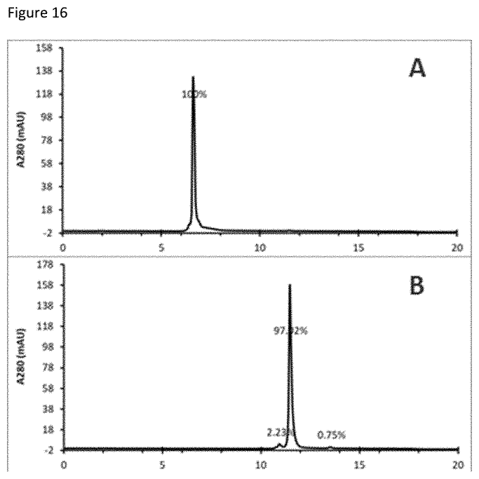

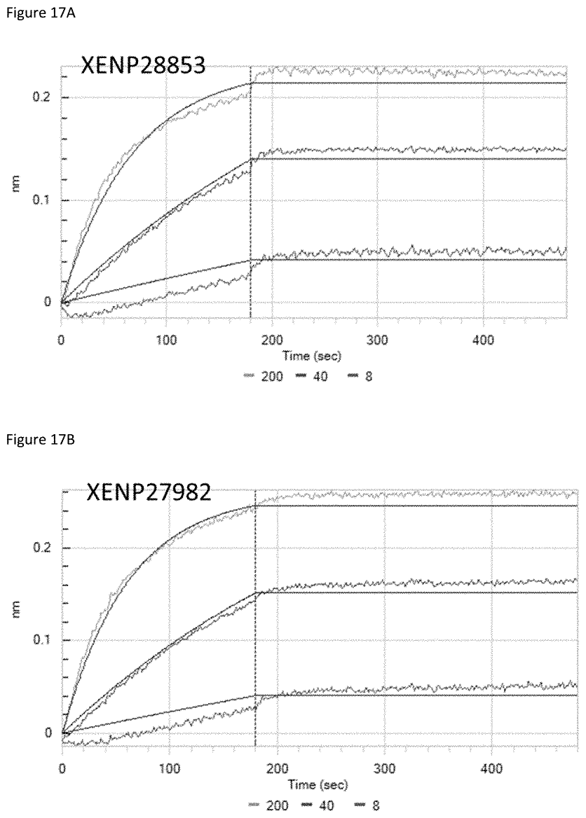

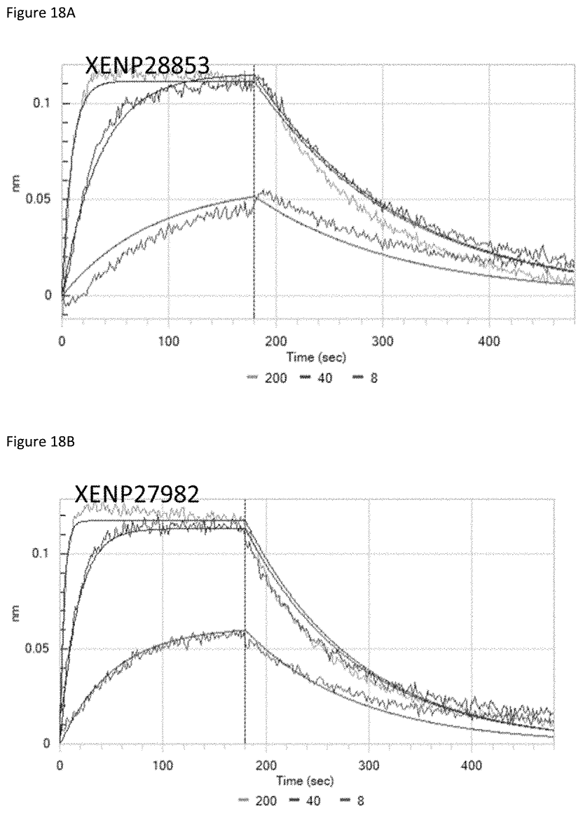

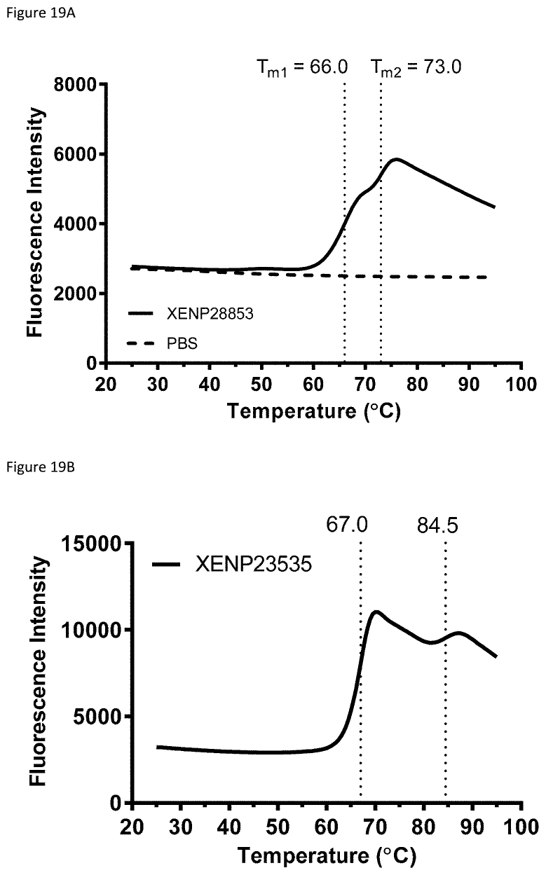

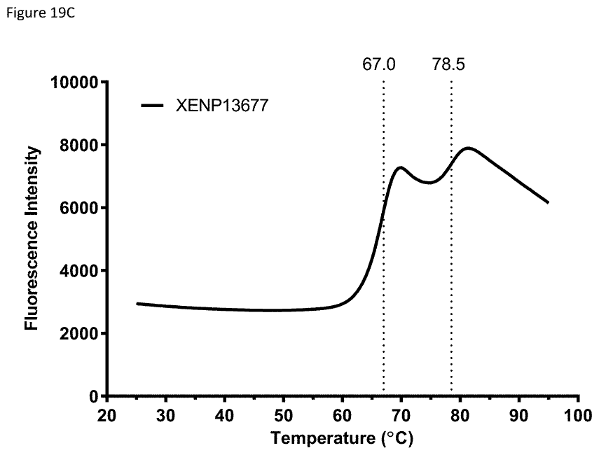

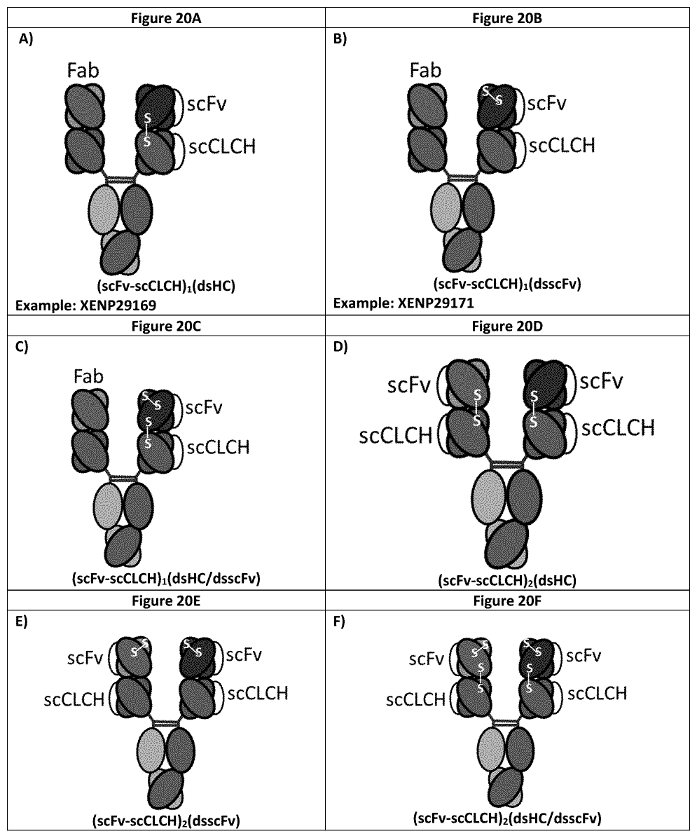

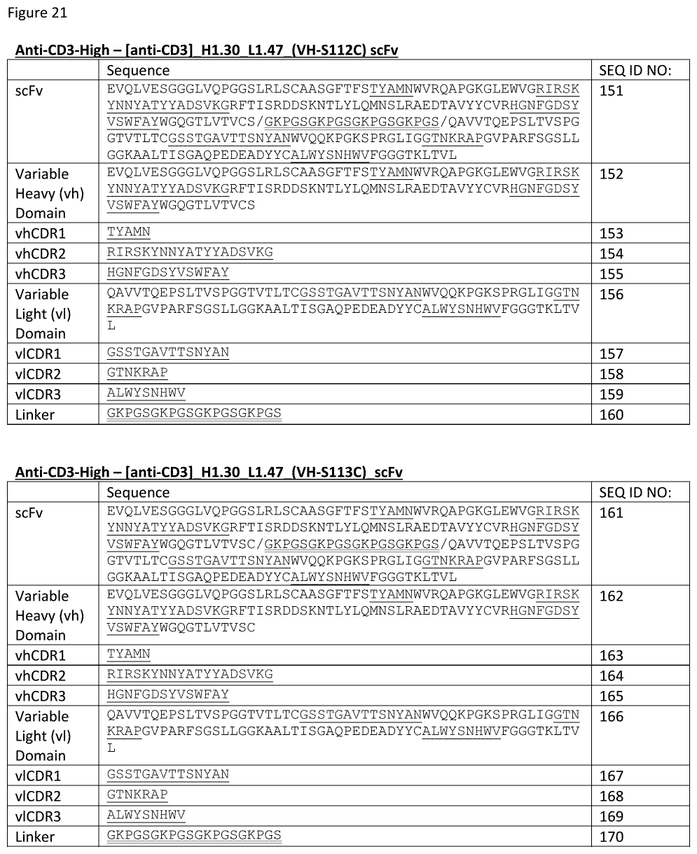

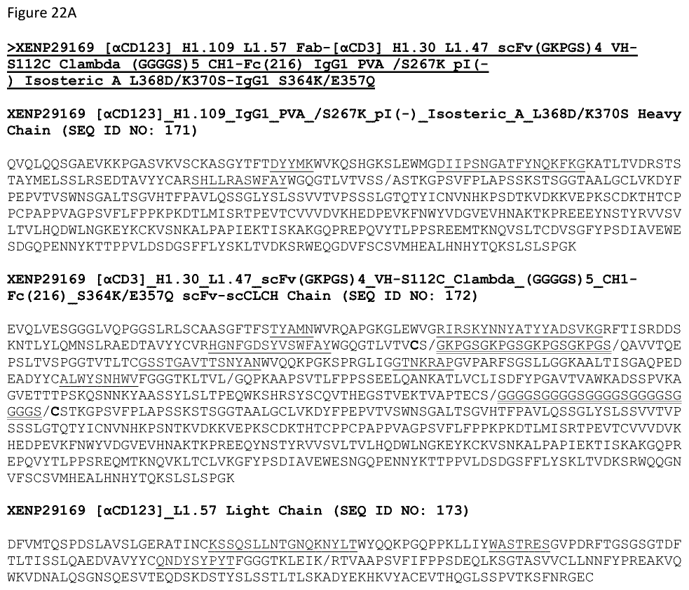

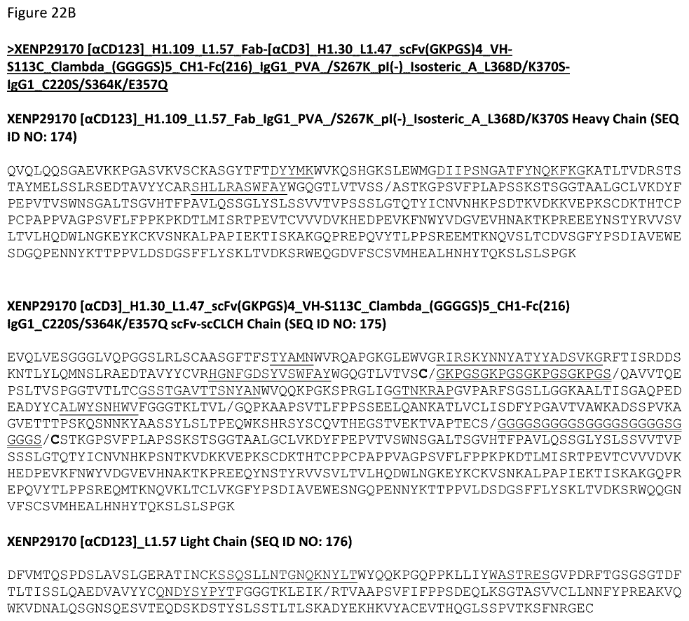

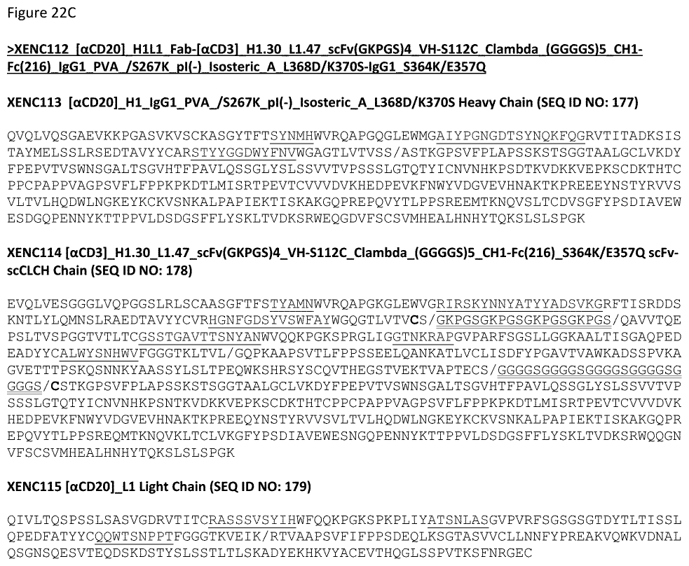

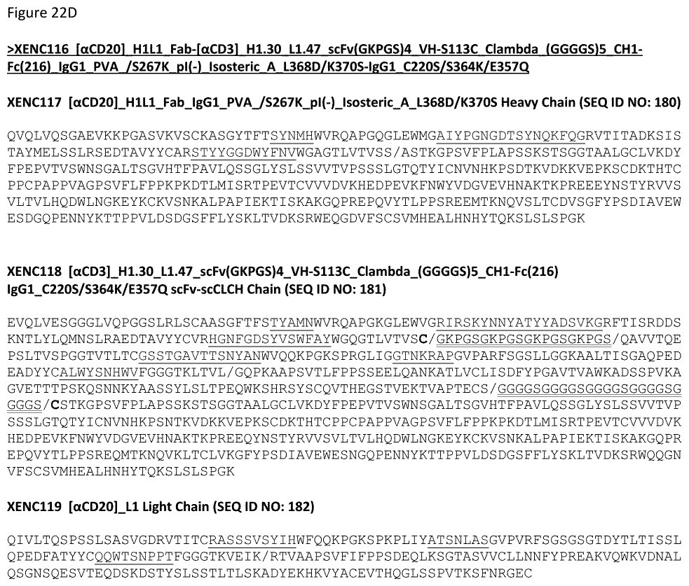

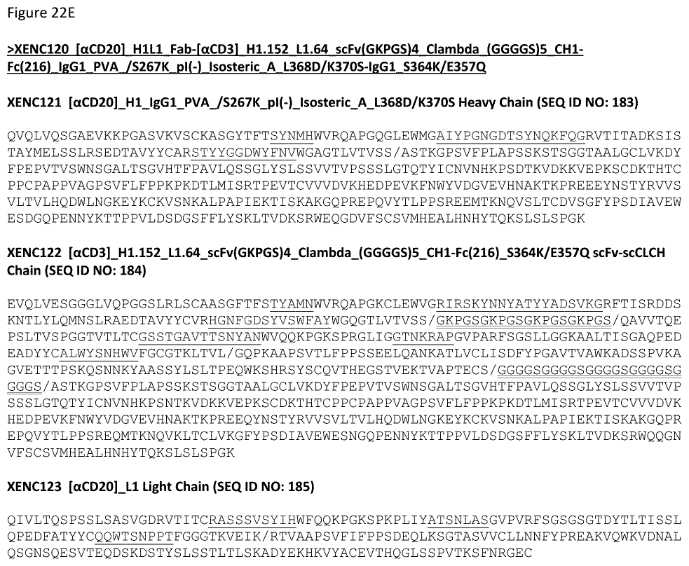

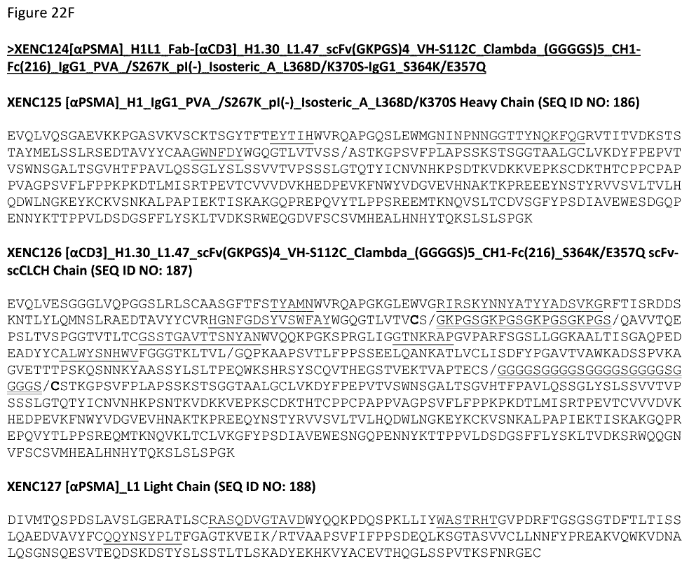

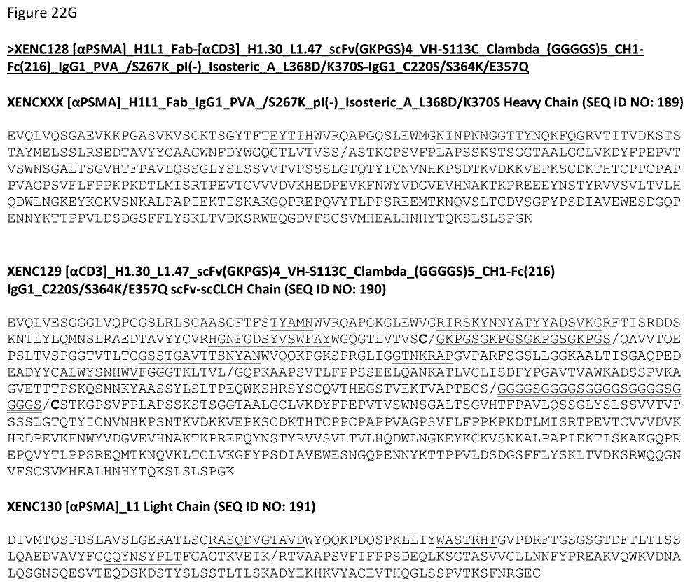

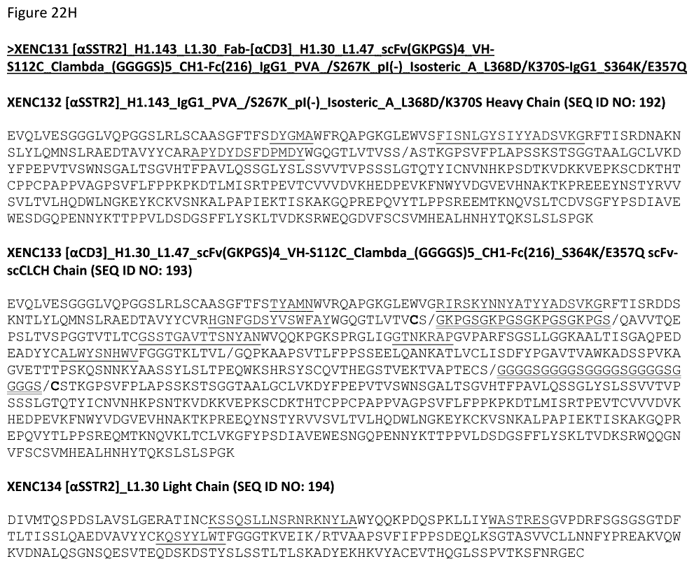

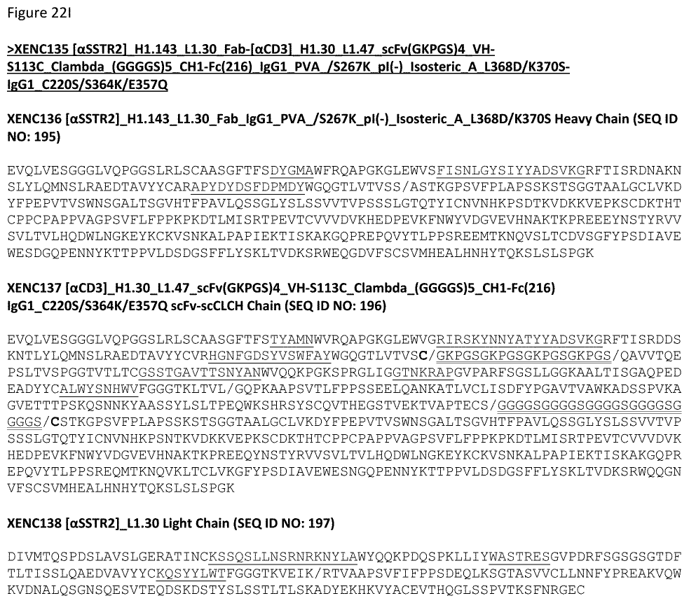

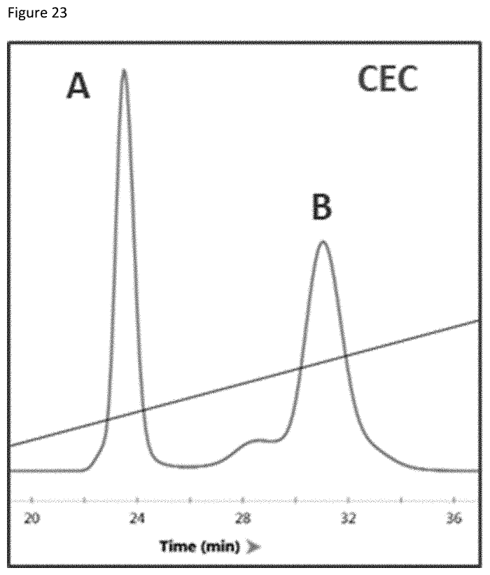

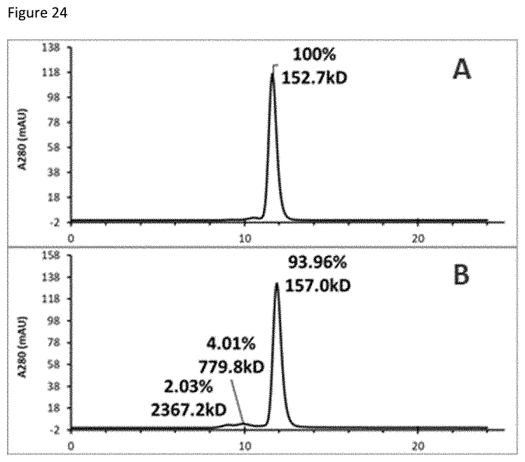

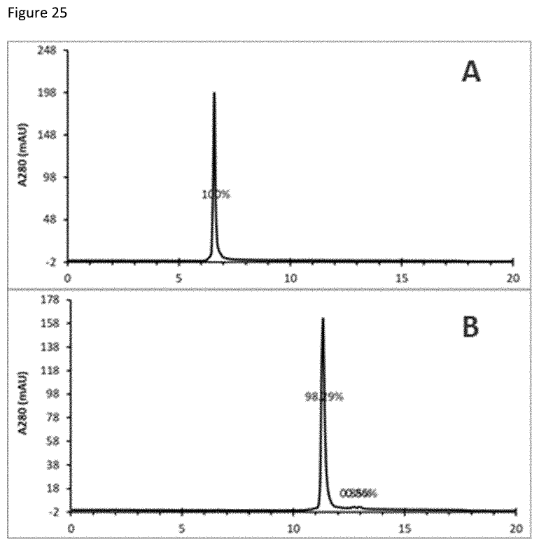

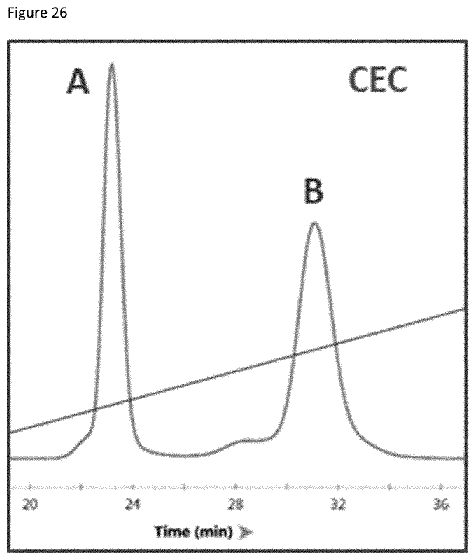

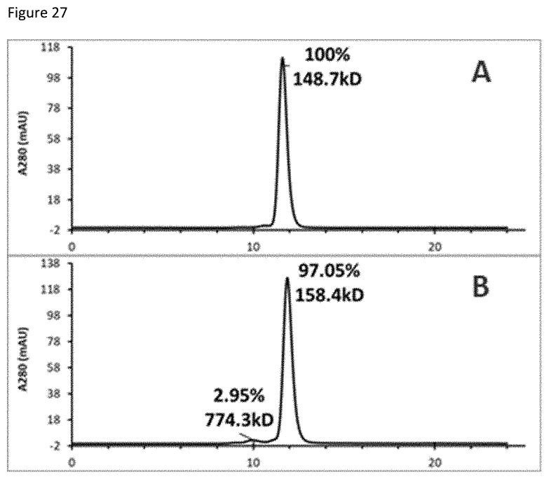

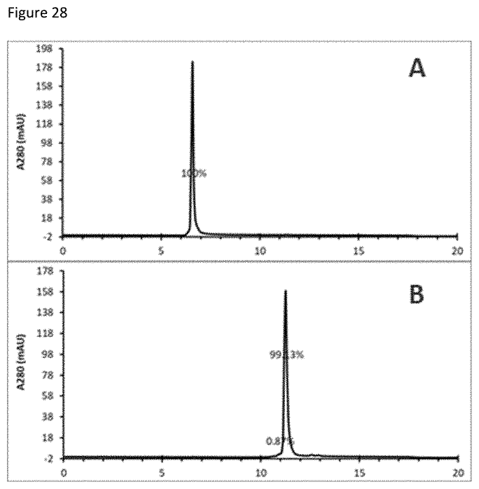

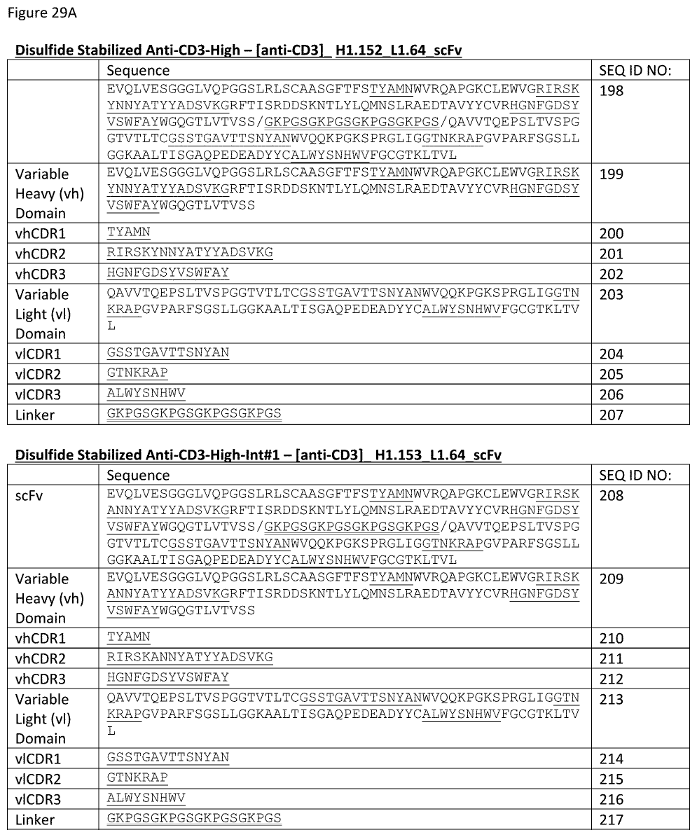

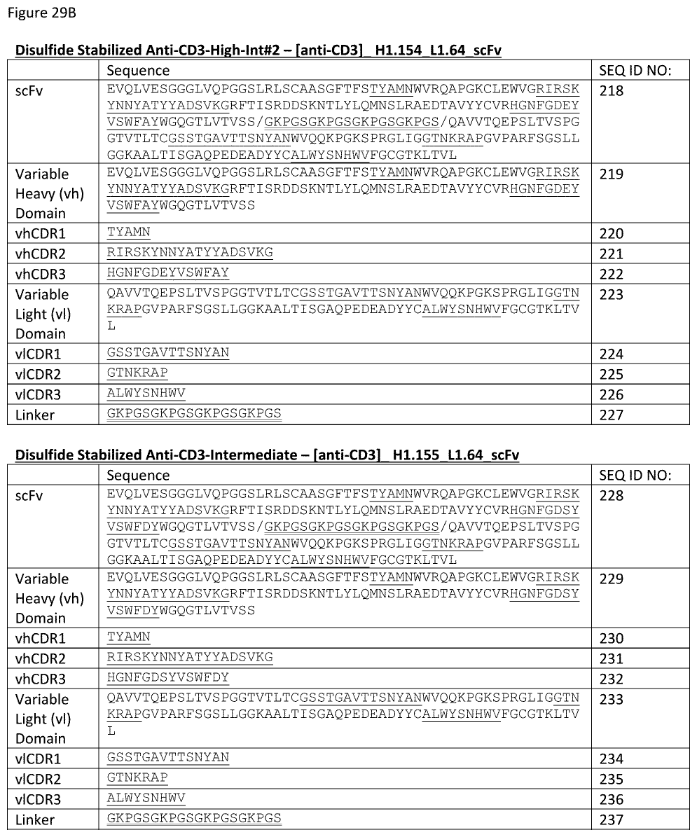

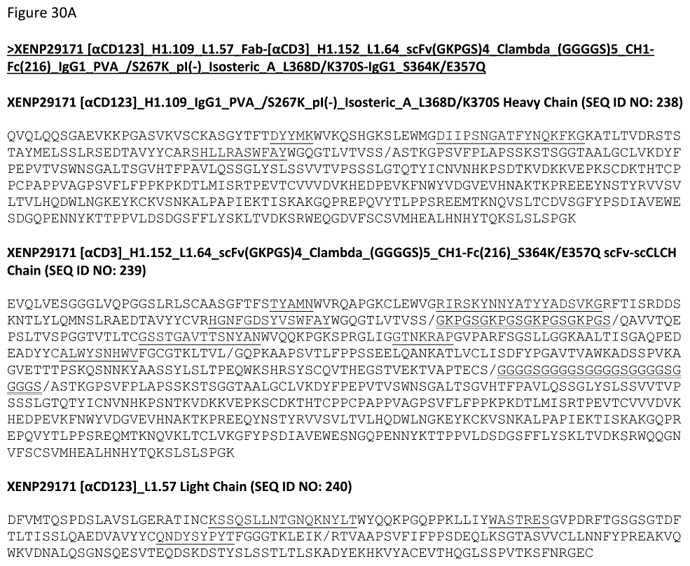

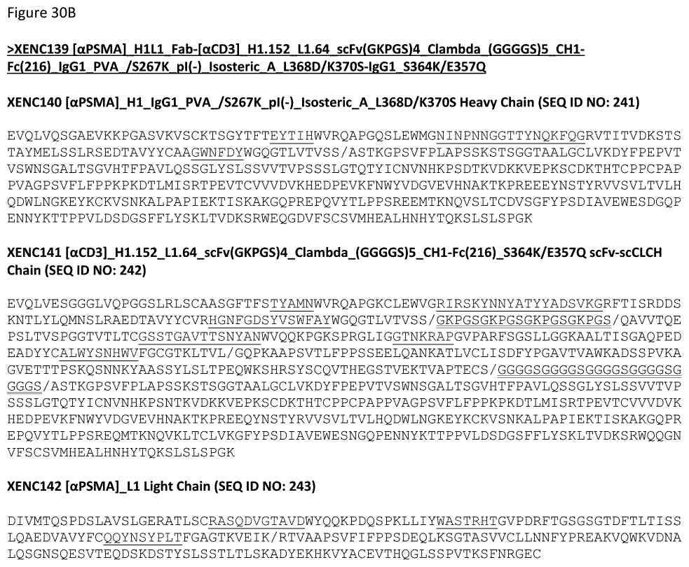

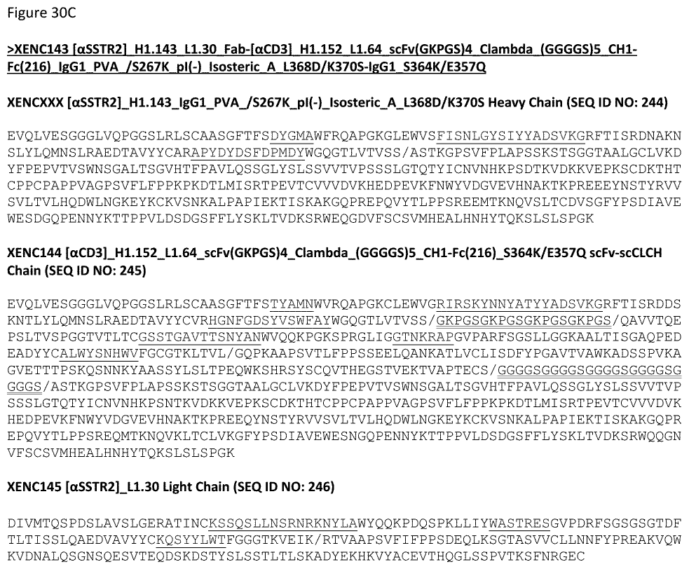

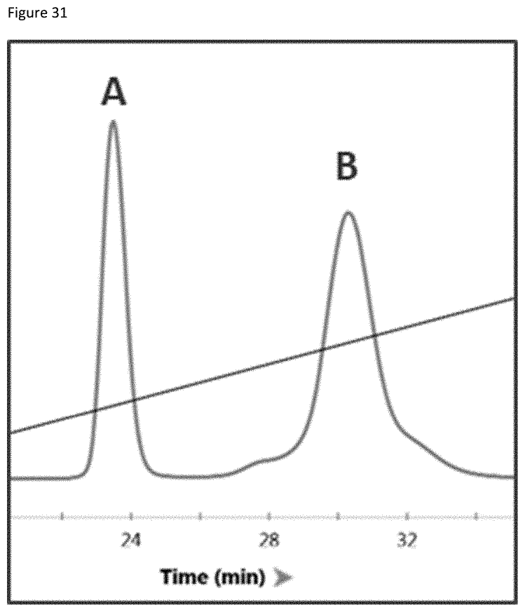

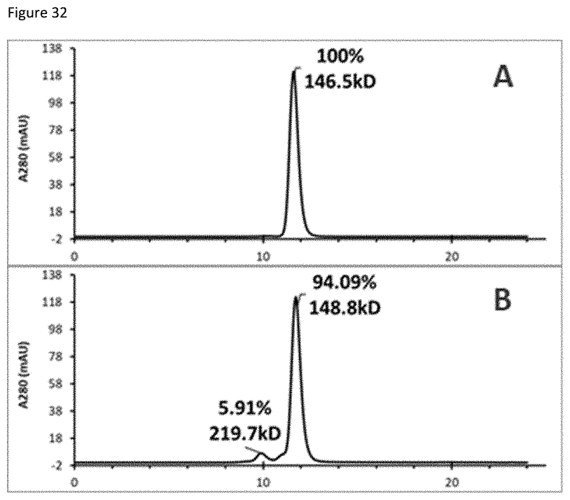

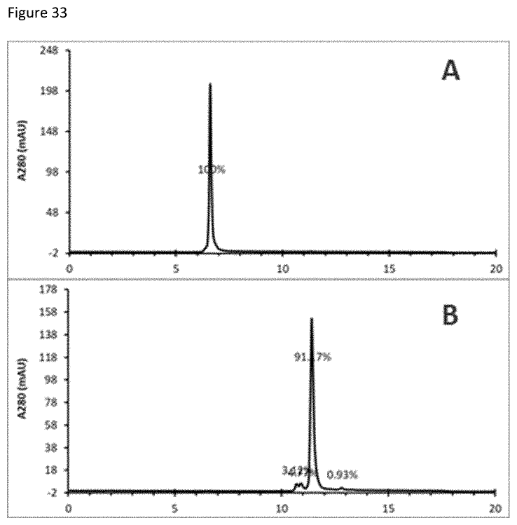

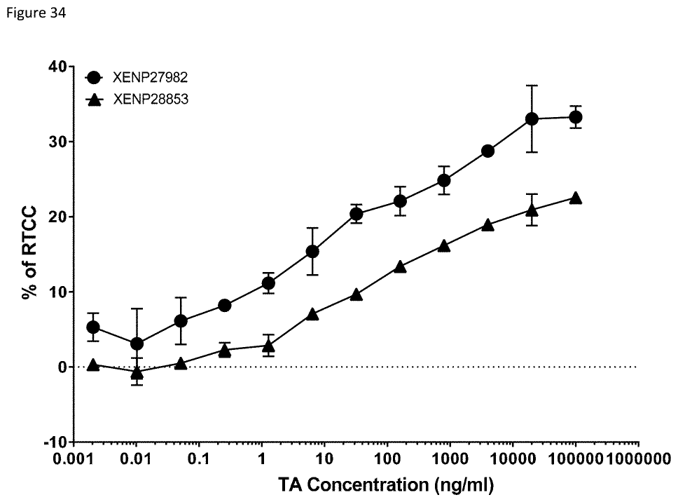

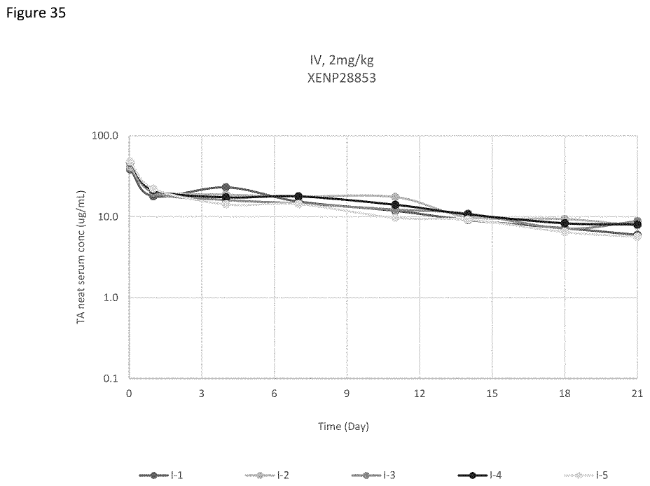

FIG. 9 depicts useful single-chain constant light/constant heavy domain (or scCLCH). These illustrative scCLCH utilize the constant heavy domain monomer 2 (+) of Backbone 1 as depicted in FIG. 7, and constant light backbone sequences as depicted in FIG. 8, and a (GGGGS)4 domain linker (SEQ ID NO: 1) as depicted in FIG. 6. As will be appreciated by those skilled in the art, included herein are scCLCH sequences that are 90, 95, 98 and 99% identical (as defined herein) to the recited sequences, and/or contain from 1, 2, 3, 4, 5, 6, 7, 8, 9 or 10 additional amino acid modifications. Furthermore, included herein are scCLCH that comprise any other constant heavy domain sequences, any other constant light backbone sequence, and/or any other domain linkers, including, but not limited to, those depicted in FIG. 6.

FIGS. 10A and 10B depict sequences of illustrative bispecific antibodies in the 1+1 Fab-scFv-Fc (a.k.a. Triple F format as described in PCT Publication No. WO 2014/110601) used as controls.

FIGS. 11A-11C depict sequences for exemplary anti-CD3 scFvs suitable for use in the bispecific antibodies of the invention. The CDRs are underlined, the scFv linker is double underlined (in the sequences, the scFv linker is a positively charged scFv (GKPGS)₄ linker (SEQ ID NO: 2), although as will be appreciated by those in the art, this linker can be replaced by other linkers, including uncharged or negatively charged linkers, some of which are depicted in FIG. 5), and the slashes indicate the border(s) of the variable domains. In addition, the naming convention illustrates the orientation of the scFv from N- to C-terminus. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 12A-12B depicts cartoon schematics for illustrative heterodimeric IgG-like bispecific antibodies (HILBAs). FIG. 12A depicts an illustrative cartoon schematic for the (scFv-scCLCH)₁ format comprising a first monomer comprising a first variable heavy region (VH1) covalently attached to a first constant heavy domain, a second monomer comprising a first variable light region (VL1) covalently attached to a first constant light domain, and a third monomer comprising a single-chain variable region (scFv) covalently attached to a single-chain constant light/constant heavy domain (scCLCH), wherein said scFv comprises a second variable heavy region (VH2) covalently attached via an scFv linker to a second variable light region (VL2), and wherein said scCLCH comprises a second constant light domain covalently attached via a domain linker to a second constant heavy domain. FIG. 12B depicts the (scFv-scCLCH)₂ format comprising a first monomer comprising a first scFv covalently attached to a first scCLCH, and a second monomer comprising a second scFv covalently attached to a second scCLCH, wherein said first scFv comprises a first variable heavy region (VH1) covalently attached via a linker to a first variable light region (VL1) and the second scFv comprises a second variable heavy region (VH2) covalently attached via a linker to a second variable light region (VL2), wherein said first scCLCH comprises a first constant light domain covalently attached via a domain linker to a first constant heavy domain and said second scCLCH comprises a second constant light domain covalently attached via a domain linker to a second constant heavy domain.

FIGS. 13A-13D depict the sequences illustrative bispecific antibodies in the HILBA format. The antibodies are named using the Fab variable region first and the scFv variable region second, separated by a dash. CDRs are underlined and slashes indicate the border(s) of the variable regions. scFv and domain linkers are double-underlined. The scFv domain has orientation (N- to C-terminus) of VH-scFv linker-VL. In addition, each sequence outlined herein can include or exclude the M428L/N434S variants in one or preferably both Fc domains, which results in longer half-life in serum. Exemplary bispecific antibodies disclosed include CD3×CD123, CD3×CD20, CD3×PSMA, and CD3×SSTR2 bispecific antibodies.

FIG. 14 depicts chromatogram illustrating purification part 2 of XENP28853 (cation exchange chromatography following protein A chromatography).

FIG. 15 depicts the separation of species in peaks A, B, and BC (purification part 2 of XENP28853 as depicted in FIG. 14) by analytical size-exclusion chromatography as well as molecular weight of component species by multi-angle light scattering (aSEC-MALS).

FIG. 16 depicts the separation of species in peaks A and B (from purification part 2 of XENP28853 as depicted in FIG. 14) by analytical cation exchange chromatography. The separation profiles show that the species in peak B elutes later than the species in peak A, indicating that the species in peak B is XENP28853.

FIGS. 17A and 17B show sensorgrams depicting binding of A) XENP28853 and B) XENP27982 to human CD123, as determined by Octet.

FIGS. 18A and 18B show sensorgrams depicting binding of XENP28853 and XENP27982 to human CD3Dε, as determined by Octet.

FIGS. 19A-19C depicts unfolding transition of A) XENP28853, B) XENP23535, and C) XENP13677 as determined by differential scanning fluorimetry.

FIGS. 20A-20F depict cartoon schematics for illustrative heterodimeric Ig-G like bispecific antibody (HILBA) format having engineered disulfide bonds. FIG. 20A depicts the (scFv-scCLCH)₁ format having disulfide stabilized heavy chain (or (scFv-scCLCH)₁ (dsHC)) comprising a first monomer comprising a first variable heavy region (VH1) covalently attached to a first constant heavy domain, a second monomer comprising a first variable light region (VL1) covalently attached to a first constant light domain, and a third monomer comprising a single-chain variable region (scFv) covalently attached to a single-chain constant light/constant heavy domain (scCLCH), wherein said scFv comprises a second variable heavy region (VH2) covalently attached via an scFv linker to a second variable light region (VL2), and wherein said scCLCH comprises a second constant light domain covalently attached via a domain linker to a second constant heavy domain; and further wherein the C-terminus of VH2 comprises a first engineered cysteine residue, and the N-terminus of the second constant heavy domain comprises a second engineered cysteine residue so that the first and second engineered cysteine residues form a disulfide bond. FIG. 20B depicts the (scFv-scCLCH)₁ format having disulfide stabilized scFv (or (scFv-scCLCH)₁(dsscFv)) comprising a first monomer comprising a first variable heavy region (VH1) covalently attached to a first constant heavy domain, a second monomer comprising a first variable light region (VL1) covalently attached to a first constant light domain, and a third monomer comprising a single-chain variable region (scFv) covalently attached to a single-chain constant light/constant heavy domain (scCLCH), wherein said scFv comprises a second variable heavy region (VH2) covalently attached via an scFv linker to a second variable light region (VL2), and wherein said scCLCH comprises a second constant light domain covalently attached via a domain linker to a second constant heavy domain; and further wherein the VH2 comprises a first engineered cysteine residue, the VL2 comprises a second engineered cysteine residue so that the first and second engineered cysteine residues form a disulfide bond. FIG. 20C depicts the (scFv-scCLCH)₁ format having disulfide stabilized heavy chain and scFv (or (scFv-scCLCH)₁(dsHC/dsscFv)) comprising a first monomer comprising a first variable heavy region (VH1) covalently attached to a first constant heavy domain, a second monomer comprising a first variable light region (VL1) covalently attached to a first constant light domain, and a third monomer comprising a single-chain variable region (scFv) covalently attached to a single-chain constant light/constant heavy domain (scCLCH), wherein said scFv comprises a second variable heavy region (VH2) covalently attached via an scFv linker to a second variable light region (VL2), and wherein said scCLCH comprises a second constant light domain covalently attached via a domain linker to a second constant heavy domain; and further wherein the VH2 comprises a first engineered cysteine residue as well as a second C-terminal engineered cysteine residue, the N-terminus of the second constant heavy domain comprises a third engineered cysteine residue, and the VL2 comprises a fourth engineered cysteine residue so that the first and fourth engineered cysteine residues and the second and the third engineered cysteine residues form disulfide bonds. FIG. 20D depicts the (scFv-scCLCH)₂ format having disulfide stabilized heavy chains (or (scFv-scCLCH)₂(dsHC)) comprising a first monomer comprising a first scFv covalently attached to a first scCLCH, and a second monomer comprising a second scFv covalently attached to a second scCLCH, wherein said first scFv comprises a first variable heavy region (VH1) covalently attached via an scFv linker to a first variable light region (VL1), and the second scFv comprises a second variable heavy region (VH2) covalently attached via an scFv linker to a second variable light region (VL2), wherein said first scCLCH comprises a first constant light domain covalently attached via a domain linker to a first constant heavy domain, and said second scCLCH comprises a second constant light domain covalently attached via a domain linker to a second constant heavy domain; and further wherein the C-terminus of VH1 comprises a first engineered cysteine residue, the N-terminus of the first constant heavy domain comprises a second engineered cysteine residue, the C-terminus of VH2 comprises a third engineered cysteine residue, and the N-terminus of the second constant heavy domain comprises a fourth engineered cysteine residue, so that the first and second engineered cysteine residues and the third and fourth engineered cysteine residues form disulfide bonds. FIG. 20E depicts the (scFv-scCLCH)₂ format having disulfide stabilized scFvs (or (scFv-scCLCH)₂(dsscFv)) comprising a first monomer comprising a first scFv covalently attached to a first scCLCH, and a second monomer comprising a second scFv covalently attached to a second scCLCH, wherein said first scFv comprises a first variable heavy region (VH1) covalently attached via an scFv linker to a first variable light region (VL1) and the second scFv comprises a second variable heavy region (VH2) covalently attached via an scFv linker to a second variable light region (VL2), wherein said first scCLCH comprises a first constant light domain covalently attached via a domain linker to a first constant heavy domain and said second scCLCH comprises a second constant light domain covalently attached via a domain linker to a second constant heavy domain; and further wherein the VH1 comprises a first engineered cysteine residue, the VL2 comprises a second engineered cysteine residue, the VH2 comprises a third engineered cysteine residue, and the VL2 comprises a fourth engineered cysteine residue so that the first and second engineered cysteine residues and the third and fourth engineered cysteine residues form disulfide bonds. FIG. 20F depicts the (scFv-scCLCH)₂ format having disulfide stabilized heavy chains and scFvs (or (scFv-scCLCH)₂(dsHC/scFv)) comprising a first monomer comprising a first scFv covalently attached to a first scCLCH, and a second monomer comprising a second scFv covalently attached to a second scCLCH, wherein said first scFv comprises a first variable heavy region (VH1) covalently attached via an scFv linker to a first variable light region (VL1) and the second scFv comprises a second variable heavy region (VH2) covalently attached via an scFv linker to a second variable light region (VL2), wherein said first scCLCH comprises a first constant light domain covalently attached via a domain linker to a first constant heavy domain, and said second scCLCH comprises a second constant light domain covalently attached via a domain linker to a second constant heavy domain; and further wherein the VH1 comprises a first engineered cysteine residue as well as a second C-terminal engineered cysteine residue, the N-terminus of the first constant heavy domain comprises a third engineered cysteine residue, the VL1 comprises a fourth engineered cysteine residue, the VH2 comprises a fifth engineered cysteine residue as well as a sixth C-terminal engineered cysteine residue, the N-terminus of the second constant heavy domain comprises a seventh engineered cysteine residue, and the VL2 comprises an eighth engineered cysteine residue so that the first and the fourth engineered cysteine residues, the second and the third engineered cysteine residues, the fifth and the eighth engineered cysteine residues, and the sixth and the seventh engineered cysteine residues form disulfide bonds.

FIG. 21 depicts sequences for exemplary anti-CD3 scFvs comprising an engineered cysteine in the C-terminus of the variable heavy region so as to form a disulfide bond with a constant heavy domain monomer in the context of, for example, the (scFv-scCLCH)₁(dsHC), (scFv-scCLCH)₁(dsHC/dsscFv), (scFv-scCLCH)₂(dsHC), or (scFv-scCLCH)₂(dsHC/dsscFv) formats. The CDRs are underlined, the scFv linker is double underlined (in the sequences, the scFv linker is a positively charged scFv (GKPGS)₄ linker (SEQ ID NO: 2), although as will be appreciated by those in the art, this linker can be replaced by other linkers, including uncharged or negatively charged linkers, some of which are depicted in FIG. 5), and the slashes indicate the border(s) of the variable domains. In addition, the naming convention illustrates the orientation of the scFv from N- to C-terminus. As noted herein and is true for every sequence herein containing CDRs, the exact identification of the CDR locations may be slightly different depending on the numbering used as is shown in Table X, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIGS. 22A-221 depict sequences for illustrative heterodimeric IgG-like bispecific antibodies (HILBAs) in the (scFv-scCLCH)₁ format with (dsHC) variants. The antibodies are named using the Fab variable region first and the scFv variable region second, separated by a dash. CDRs are underlined and slashes indicate the border(s) of the variable regions. scFv and domain linkers are double-underlined. Engineered cysteine residues are in bold. The scFv domain has orientation (N- to C-terminus) of VH-scFv linker-VL. In addition, each sequence outlined herein can include or exclude the M428L/N434S variants in one or preferably both Fc domains, which results in longer half-life in serum. The exemplary antibodies depicted include CD3×CD123, CD3×CD20, CD3×PSMA, and CD3×SSTR2 bispecific antibodies.

FIG. 23 depicts chromatogram illustrating purification part 2 of XENP29169 (cation exchange chromatography following protein A chromatography).

FIG. 24 depicts the separation of species in peaks A and B (purification part 2 of XENP29169 as depicted in FIG. 23) by analytical size-exclusion chromatography as well as molecular weight of component species by multi-angle light scattering (aSEC-MALS).

FIG. 25 depicts the separation of species in peaks A and B (from purification part 2 of XENP29169 as depicted in FIG. 23) by analytical cation exchange chromatography. The separation profiles show that the species in peak B elutes later than the species in peak A, indicating that the species in peak B is XENP29169.

FIG. 26 depicts chromatogram illustrating purification part 2 of XENP29170 (cation exchange chromatography following protein A chromatography).

FIG. 27 depicts the separation of species in peaks A and B (purification part 2 of XENP29170 as depicted in FIG. 26) by analytical size-exclusion chromatography as well as molecular weight of component species by multi-angle light scattering (aSEC-MALS).

FIG. 28 depicts the separation of species in peaks A and B (from purification part 2 of XENP29170 as depicted in FIG. 26) by analytical cation exchange chromatography. The separation profiles show that the species in peak B elutes later than the species in peak A, indicating that the species in peak B is XENP29170.

FIGS. 29A and 29B depict illustrative disulfide stabilized anti-CD3 scFvs suitable for use in the bispecific antibodies of the invention. The CDRs are underlined, the scFv linker is double underlined (in the sequences, the scFv linker is a positively charged scFv (GKPGS)₄ linker (SEQ ID NO: 2), although as will be appreciated by those in the art, this linker can be replaced by other linkers, including uncharged or negatively charged linkers, some of which are depicted in FIG. 5), and the slashes indicate the border(s) of the variable domains. In addition, the naming convention illustrates the orientation of the scFv from N- to C-terminus. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIGS. 30A-30C depict sequences for illustrative bispecific antibodies in the heterodimeric IgG-like bispecific antibodies (HILBAs) in the (scFv-scCLCH)₁ format with (dsscFv) variants. The antibodies are named using the Fab variable region first and the scFv variable region second, separated by a dash. CDRs are underlined and slashes indicate the border(s) of the variable regions. scFv and domain linkers are double-underlined. Engineered cysteine residues are in bold. The scFv domain has orientation (N- to C-terminus) of VH-scFv linker-VL. In addition, each sequence outlined herein can include or exclude the M428L/N434S variants in one or preferably both Fc domains, which results in longer half-life in serum.

FIG. 31 depicts chromatogram illustrating purification part 2 of XENP29171 (cation exchange chromatography following protein A chromatography).

FIG. 32 depicts the separation of species in peaks A and B (purification part 2 of XENP29171 as depicted in FIG. 31) by analytical size-exclusion chromatography as well as molecular weight of component species by multi-angle light scattering (aSEC-MALS).

FIG. 33 depicts the separation of species in peaks A and B (from purification part 2 of XENP29171 as depicted in FIG. 31) by analytical cation exchange chromatography. The separation profiles show that the species in peak B elutes later than the species in peak A, indicating that the species in peak B is XENP29171.

FIG. 34 depicts the result of a redirected T cell cytotoxicity (RTCC) assay, using XENP27982 (circles) and XENP28853 (triangles) with KG1a cells. The data show that both XENP27982 and XENP28853 induced killing of KG1a cells.

FIG. 35 depicts the serum concentration of XENP28853 over time in 5 individual C57BL/6J mice after dosing with 2 mg/kg of XENP28853. Half-life estimation based on the average serum concentration and data from Days 4 to 21 was 13.5 days.

### DETAILED DESCRIPTION

### I. Overview

Provided herein are novel Heterodimeric IgG-Like Bispecific Antibodies (HILBAs). Such bispecific antibodies are similar to IgG in structure and, thus, do not exhibit a limited half-life like previous antibody fragment-based bispecifics. Moreover, such heterodimeric IgG-like bispecific antibodies advantageously do not entail complicated generation methods such as Fab arm exchange and common light chain methods.

Similar to the canonical IgG antibody structure, the heterodimeric IgG-like bispecific antibodies provided herein each include two variable heavy domains (VH), two variable light domains (VL), two variable constant domains (CL), and two CH1-hinge-CH2-CH3 monomers. Thus, the heterodimeric IgG-like bispecific antibodies provided herein do not exhibit a limited half-life like antibody fragment-based bispecific counterparts.

These heterodimeric IgG-like bispecific antibodies, however, include at least one monomer that includes, from N- to C-terminus: a VH-scFv linker-VL-CL-domain linker-CH1-hinge-CH2-CH3. This monomer facilitates bispecific antibody generation over more complicated methods such as, for example, bispecific antibody generation Fab arm exchange and common light chain methods.

Aspects of the subject heterodimeric IgG-like bispecific antibodies are described in greater detail below.

### II. Definitions

In order that the application may be more completely understood, several definitions are set forth below. Such definitions are meant to encompass grammatical equivalents.

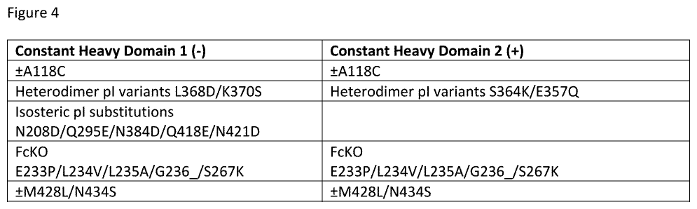

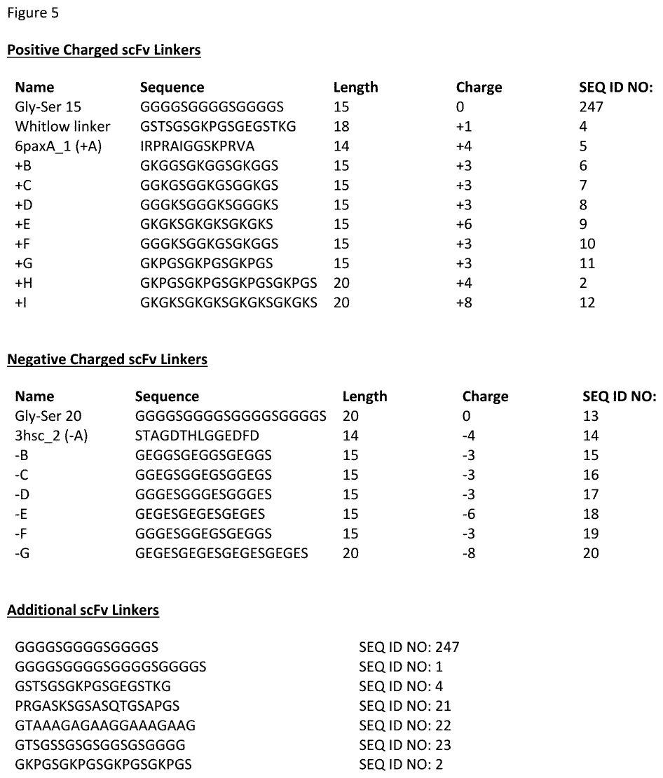

By “ablation” herein is meant a decrease or removal of binding and/or activity. Thus for example, “ablating FcγR binding” means the Fc region amino acid variant has less than 50% starting binding as compared to an Fc region not containing the specific variant, with more than 70-80-90-95-98% loss of binding being preferred, and in general, with the binding being below the level of detectable binding in a Biacore assay. Of particular use in the ablation of FcγR binding are those shown in FIG. 4. However, unless otherwise noted, the Fc monomers of the invention retain binding to the FcRn.

By “ADCC” or “antibody dependent cell-mediated cytotoxicity” as used herein is meant the cell-mediated reaction wherein nonspecific cytotoxic cells that express FzzcγRs recognize bound antibody on a target cell and subsequently cause lysis of the target cell. ADCC is correlated with binding to FcγRIIIa; increased binding to FcγRIIIa leads to an increase in ADCC activity. As is discussed herein, some embodiments ablate ADCC activity entirely.

By “modification” herein is meant an amino acid substitution, insertion, and/or deletion in a polypeptide sequence or an alteration to a moiety chemically linked to a protein. For example, a modification may be an altered carbohydrate or PEG structure attached to a protein. By “amino acid modification” herein is meant an amino acid substitution, insertion, and/or deletion in a polypeptide sequence. For clarity, unless otherwise noted, the amino acid modification is always to an amino acid coded for by DNA, e.g., the 20 amino acids that have codons in DNA and RNA.

By “amino acid substitution” or “substitution” herein is meant the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For example, the substitution E272Y or 272Y refers to a variant polypeptide, in this case an Fc variant, in which the glutamic acid at position 272 is replaced with tyrosine. For clarity, a protein which has been engineered to change the nucleic acid coding sequence but not to change the starting amino acid (for example exchanging CGG (encoding arginine) to CGA (still encoding arginine) to increase host organism expression levels) is not an “amino acid substitution”; that is, despite the creation of a new gene encoding the same protein, if the protein has the same amino acid at the particular position that it started with, it is not an amino acid substitution.

By “amino acid insertion” or “insertion” as used herein is meant the addition of an amino acid residue or sequence at a particular position in a parent polypeptide sequence. For example, -233E designates an insertion of glutamic acid after position 233 and before position 234. Additionally, -233ADE or A233ADE designates an insertion of AlaAspGlu after position 233 and before position 234.

By “amino acid deletion” or “deletion” as used herein is meant the removal of an amino acid residue or sequence at a particular position in a parent polypeptide sequence. For example, E233-, E233#, E233( ), E233_, or E233del designates a deletion of glutamic acid at position 233. Additionally, EDA233- or EDA233# designates a deletion of the sequence GluAspAla that begins at position 233.

By “variant protein”, “protein variant”, or “variant” as used herein is meant a protein that differs from that of a parent protein by virtue of at least one modification. Protein variant may refer to the protein itself, a composition comprising the protein, the amino acid sequence that encodes it, or the DNA sequence that encodes it. Preferably, the protein variant has at least one amino acid modification compared to the parent protein, e.g. from about one to about seventy amino acid modifications, and preferably from about one to about five amino acid modifications compared to the parent. The modification can be an addition, deletion, or substitution. As described below, in some embodiments the parent protein, for example an Fc parent polypeptide, is a human wild type sequence, such as the Fc region from IgG1, IgG2, IgG3 or IgG4. The protein variant sequence herein will preferably possess at least about 80% identity with a parent protein sequence, and most preferably at least about 90% identity, more preferably at least about 95-98-99% identity. “Variant,” as used herein can also refer to particular amino acid modifications (e.g., substitutions, deletions, insertions) in a variant protein (e.g., a variant Fc domain), for example, heterodimerization variants, ablation variants, FcKO variants, etc., as disclosed in Section III below.

As used herein, by “protein” is meant at least two covalently attached amino acids, which includes proteins, polypeptides, oligopeptides and peptides. When a biologically functional molecule comprises two or more proteins, each protein may be referred to as a “monomer” or as a “subunit; and the biologically functional molecule may be referred to as a “complex.”

By “residue” as used herein is meant a position in a protein and its associated amino acid identity. For example, Asparagine 297 (also referred to as Asn297 or N297) is a residue at position 297 in the human antibody IgG1.

By “IgG subclass modification” or “isotype modification” as used herein is meant an amino acid modification that converts one amino acid of one IgG isotype to the corresponding amino acid in a different, aligned IgG isotype. For example, because IgG1 comprises a tyrosine and IgG2 a phenylalanine at EU position 296, a F296Y substitution in IgG2 is considered an IgG subclass modification.

By “non-naturally occurring modification” as used herein with respect to an IgG domain is meant an amino acid modification that is not isotypic. For example, because none of the IgGs comprise a serine at position 434, the substitution 434S in IgG1, IgG2, IgG3, or IgG4 (or hybrids thereof) is considered a non-naturally occurring modification.

By “amino acid” and “amino acid identity” as used herein is meant one of the 20 naturally occurring amino acids that are coded for by DNA and RNA.

By “effector function” as used herein is meant a biochemical event that results from the interaction of an antibody Fc region with an Fc receptor or ligand. Effector functions include but are not limited to ADCC, ADCP, and CDC.

By “IgG Fc ligand” or “Fc ligand” as used herein is meant a molecule, preferably a polypeptide, from any organism that binds to the Fc region of an IgG antibody to form an Fc/Fc ligand complex. Fc ligands include but are not limited to FcγRIs, FcγRIIs, FcγRIIIs, FcRn, C1q, C3, mannan binding lectin, mannose receptor, staphylococcal protein A, streptococcal protein G, and viral FcγR. Fc ligands also include Fc receptor homologs (FcRH), which are a family of Fc receptors that are homologous to the FcγRs (Davis et al., 2002, Immunological Reviews 190:123-136, entirely incorporated by reference). Fc ligands may include undiscovered molecules that bind Fc. Particular IgG Fc ligands are FcRn and Fc gamma receptors.

By “Fc gamma receptor”, “FcγR” or “FcgammaR” as used herein is meant any member of the family of proteins that bind the IgG antibody Fc region and is encoded by an FcγR gene. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIb-NA1 and FcγRIIb-NA2) (Jefferis et al., 2002, Immunol Lett 82:57-65, entirely incorporated by reference), as well as any undiscovered human FcγRs or FcγR isoforms or allotypes. An FcγR may be from any organism, including but not limited to humans, mice, rats, rabbits, and monkeys. Mouse FcγRs include but are not limited to FcγRI (CD64), FcγRII (CD32), FcγRIII (CD16), and FcγRIII-2 (CD16-2), as well as any undiscovered mouse FcγRs or FcγR isoforms or allotypes.

By “FcRn” or “neonatal Fc receptor” as used herein is meant a protein that binds the IgG antibody Fc region and is encoded at least in part by an FcRn gene. The FcRn may be from any organism, including but not limited to humans, mice, rats, rabbits, and monkeys. As is known in the art, the functional FcRn protein comprises two polypeptides, often referred to as the heavy chain and light chain. The light chain is beta-2-microglobulin (β2-microglobulin) and the heavy chain is encoded by the FcRn gene. Unless otherwise noted herein, FcRn or an FcRn protein refers to the complex of FcRn heavy chain with β2-microglobulin. A variety of Fc variants can be used to increase binding to the FcRn, and in some cases, to increase serum half-life. In general, unless otherwise noted, the Fc monomers of the invention retain binding to the FcRn (and, as noted below, can include amino acid variants to increase binding to the FcRn).

By “parent polypeptide” as used herein is meant a starting polypeptide that is subsequently modified to generate a variant. The parent polypeptide may be a naturally occurring polypeptide (i.e., a wildtype polypeptide), or a variant or engineered version of a naturally occurring polypeptide. Parent polypeptide may refer to the polypeptide itself, compositions that comprise the parent polypeptide, or the amino acid sequence that encodes it.

By “Fc” or “Fc region” or “Fc domain” as used herein is meant the polypeptide comprising the constant region of an antibody, in some instances, excluding all of the first constant region immunoglobulin domain (e.g., CH1) or a portion thereof, and in some cases, optionally including all or part of the hinge. For IgG, the Fc domain comprises immunoglobulin domains CH2 and CH3 (Cγ2 and Cγ3), and optionally all or a portion of the hinge region between CH1 (Cγ1) and CH2 (Cγ2). Thus, in some cases, the Fc domain includes, from N- to C-terminus, CH2-CH3 and hinge-CH2-CH3. In some embodiments, the Fc domain is that from IgG1, IgG2, IgG3 or IgG4, with IgG1 hinge-CH2-CH3 and IgG4 hinge-CH2-CH3 finding particular use in many embodiments. Additionally, in certain embodiments, wherein the Fc domain is a human IgG1 Fc domain, the hinge includes a C220S amino acid substitution. Furthermore, in some embodiments where the Fc domain is a human IgG4 Fc domain, the hinge includes a S228P amino acid substitution. Although the boundaries of the Fc region may vary, the human IgG heavy chain Fc region is usually defined to include residues E216, C226, or A231 to its carboxyl-terminus, wherein the numbering is according to the EU index as in Kabat. In some embodiments, as is more fully described below, amino acid modifications are made to the Fc region, for example to alter binding to one or more FcγR or to the FcRn.

As will be appreciated by those in the art, the exact numbering and placement of the heavy constant region domains can be different among different numbering systems. A useful comparison of heavy constant region numbering according to EU and Kabat is as below, see Edelman et al., 1969, Proc Natl Acad Sci USA 63:78-85 and Kabat et al., 1991, Sequences of Proteins of Immunological Interest, 5th Ed., United States Public Health Service, National Institutes of Health, Bethesda, entirely incorporated by reference.

TABLE 1

| | EU Numbering | Kabat Numbering |
|---|---|---|
| CH1 | 118-215 | 114-223 |
| Hinge | 216-230 | 226-243 |
| CH2 | 231-340 | 244-360 |
| CH3 | 341-447 | 361-478 |

“Fc variant” or “variant Fc” as used herein is meant a protein comprising an amino acid modification in an Fc domain. The modification can be an addition, deletion, or substitution. The Fc variants of the present invention are defined according to the amino acid modifications that compose them. Thus, for example, N434S or 434S is an Fc variant with the substitution for serine at position 434 relative to the parent Fc polypeptide, wherein the numbering is according to the EU index. Likewise, M428L/N434S defines an Fc variant with the substitutions M428L and N434S relative to the parent Fc polypeptide. The identity of the WT amino acid may be unspecified, in which case the aforementioned variant is referred to as 428L/434S. It is noted that the order in which substitutions are provided is arbitrary, that is to say that, for example, 428L/434S is the same Fc variant as 434S/428L, and so on. For all positions discussed herein that relate to antibodies or derivatives and fragments thereof (e.g., Fc domains), unless otherwise noted, amino acid position numbering is according to the EU index. The “EU index” or “EU index as in Kabat” or “EU numbering” scheme refers to the numbering of the EU antibody (Edelman et al., 1969, Proc Natl Acad Sci USA 63:78-85, hereby entirely incorporated by reference). The modification can be an addition, deletion, or substitution.

By “position” as used herein is meant a location in the sequence of a protein. Positions may be numbered sequentially, or according to an established format, for example the EU index for numbering of antibody domains (e.g., a CH1, CH2, CH3 or hinge domain).

By “strandedness” in the context of the monomers of the heterodimeric proteins of the invention herein is meant that, similar to the two strands of DNA that “match”, heterodimerization variants are incorporated into each monomer so as to preserve, create, and/or enhance the ability to “match” to form heterodimers. For example, if some pI variants are engineered into monomer A (e.g. making the pI higher), then steric variants that are “charge pairs” that can be utilized as well do not interfere with the pI variants, e.g. the charge variants that make a pI higher are put on the same “strand” or “monomer” to preserve both functionalities. Similarly, for “skew” variants that come in pairs of a set as more fully outlined below, the skilled artisan will consider pI in deciding into which strand or monomer that incorporates one set of the pair will go, such that pI separation is maximized using the pI of the skews as well.

By “wild type,” “wildtype” or WT” herein is meant an amino acid sequence or a nucleotide sequence that is found in nature, including allelic variations. A WT protein has an amino acid sequence or a nucleotide sequence that has not been intentionally modified.

The heterodimeric IgG-like bispecific antibodies provided herein are generally isolated or recombinant. “Isolated,” when used to describe the various polypeptides disclosed herein, means a polypeptide that has been identified and separated and/or recovered from a cell or cell culture from which it was expressed. Ordinarily, an isolated polypeptide will be prepared by at least one purification step. An “isolated protein,” refers to a protein which is substantially free of other proteins from a cell culture such as host cell proteins. “Recombinant” means the proteins are generated using recombinant nucleic acid techniques in exogeneous host cells.

“Percent (%) amino acid sequence identity” with respect to a protein sequence is defined as the percentage of amino acid residues in a candidate sequence that are identical with the amino acid residues in the specific (parental) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. One particular program is the ALIGN-2 program outlined at paragraphs [0279] to [0280] of US Publ. App. No. 20160244525, hereby incorporated by reference.

The degree of identity between an amino acid sequence provided herein (“invention sequence”) and the parental amino acid sequence is calculated as the number of exact matches in an alignment of the two sequences, divided by the length of the “invention sequence,” or the length of the parental sequence, whichever is the shortest. The result is expressed in percent identity.

In some embodiments, two or more amino acid sequences are at least 50%, 60%, 70%, 80%, or 90% identical. In some embodiments, two or more amino acid sequences are at least 95%, 97%, 98%, 99%, or even 100% identical.

By “fused” or “covalently linked” is herein meant that the components (e.g., VH, VL, Fc domains) are linked by peptide bonds, either directly or indirectly via domain linkers, outlined herein.

The strength, or affinity, of specific binding can be expressed in terms of dissociation constant (KD) of the interaction, wherein a smaller KD represents greater affinity and a larger KD represents lower affinity. Binding properties can be determined by methods well known in the art such as bio-layer interferometry and surface plasmon resonance based methods. One such method entails measuring the rates of antigen-binding site/antigen or receptor/ligand complex association and dissociation, wherein rates depend on the concentration of the complex partners, the affinity of the interaction, and geometric parameters that equally influence the rate in both directions. Thus, both the association rate (ka) and the dissociation rate (kd) can be determined, and the ratio of kd/ka is equal to the dissociation constant KD (See Nature 361:186-187 (1993) and Davies et al. (1990) Annual Rev Biochem 59:439-473).

Specific binding for a particular molecule or an epitope can be exhibited, for example, by a molecule (e.g., a CD3 binding domain or a tumor target antigen binding domain) having a KD for its binding partner (e.g., an CD3 or tumor target antigen) of at least about 10⁻⁴ M, at least about 10⁻⁵M, at least about 10⁻⁶ M, at least about 10⁻⁷ M, at least about 10⁻⁸ M, at least about 10⁻⁹ M, alternatively at least about 10⁻¹⁰ M, at least about 10⁻¹¹M, at least about 10⁻¹² M, or greater. Typically, an antigen binding molecule that specifically binds an antigen will have a KD that is 20-, 50-, 100-, 500-, 1000-, 5,000-, 10,000- or more times greater for a control molecule relative to the antigen or epitope.

### III. Heterodimeric IgG-Like Bispecific Antibodies

Provided herein are novel Heterodimeric IgG-Like Bispecific Antibodies (HILBA). Such bispecific antibodies are similar to IgG in structure and, thus, do not exhibit a limited half-life like previous antibody fragment-based bispecifics. Moreover, such heterodimeric IgG-like bispecific antibodies advantageously do not entail complicated generation methods such as Fab arm exchange and common light chain methods.

Unlike canonical IgG antibodies, the HILBAs described herein include at least one monomer that is from N- to C-terminus: a VH-scFv linker-VL-CL-domain linker-CH1-hinge-CH2-CH3. With respect to this monomer, the VH-scFv linker-VL is an scFv that binds a particular antigen (e.g., CD3 or a tumor target antigen) and the CL-domain linker-CH1 forms a “single chain constant light domain/constant heavy domain” or “scCLCH”.

In one embodiment of the HILBA, termed “(scFv-scCLCH)₁,” the HILBA also includes a VH-CH1-hinge-CH2-CH3 monomer and a light chain monomer that includes a VL and CL. The VH of the VH-CH1-hinge-CH2-CH3 monomer and the VL of the light chain monomer form a binding domain that binds an antigen different that the antigen bound by the scFv.

In another embodiment of the HILBA, termed “(scFv-scCLCH)₂,” the HILBA includes a second monomer that also includes, from N- to C-terminus: a VH-scFv linker-VL-CL-domain linker-CH1-hinge-CH2-CH3. The VH-scFv linker-VL of this second monomer form a second scFv that binds an antigen different than the antigen bound by the first scFv.

The Fc domains (CH2-CH3) of the HILBA can be derived from IgG Fc domains, e.g., IgG1, IgG2, IgG3 or IgG4 Fc domains, with IgG1 Fc domains finding particular use in the invention. As described herein, IgG1 Fc domains may be used, often, but not always in conjunction with ablation variants to ablate effector function. Similarly, when low effector function is desired, IgG4 Fc domains may be used.

For any of the HILBAs described herein, the carboxy-terminal portion of each chain defines a constant region primarily responsible for effector function. Kabat et al. collected numerous primary sequences of the variable regions of heavy chains and light chains. Based on the degree of conservation of the sequences, they classified individual primary sequences into the CDRs and the framework and made a list thereof (see SEQUENCES OF IMMUNOLOGICAL INTEREST, 5th edition, NIH publication, No. 91-3242, E. A. Kabat et al., entirely incorporated by reference). Throughout the present specification, the Kabat numbering system is generally used when referring to a residue in the variable domain (approximately, residues 1-107 of the light chain variable region and residues 1-113 of the heavy chain variable region) and the EU numbering system for Fc regions (e.g., Kabat et al., supra (1991)).

In the IgG subclass of immunoglobulins, there are several immunoglobulin domains in the heavy chain. By “immunoglobulin (Ig) domain” herein is meant a region of an immunoglobulin having a distinct tertiary structure. Of interest in the present HILBAs are the heavy chain domains, including, the constant heavy (CH) domains and the hinge domains. In the context of IgG antibodies, the IgG isotypes each have three CH regions. Accordingly, “CH” domains in the context of IgG are as follows: “CH1” refers to positions 118-215 according to the EU index as in Kabat. “Hinge” refers to positions 216-230 according to the EU index as in Kabat. “CH2” refers to positions 231-340 according to the EU index as in Kabat, and “CH3” refers to positions 341-447 according to the EU index as in Kabat. Together the CH1-hinge-Fc domain or CH1-hinge-CH2-CH3 is referred to a “heavy chain constant domain,” “heavy constant domain,” or “constant heavy domain.” As shown in Table 1, the exact numbering and placement of the heavy chain domains can be different among different numbering systems. As shown herein and described below, the pI variants can be in one or more of the CH regions, as well as the hinge region, discussed below.

By “hinge” or “hinge region” or “antibody hinge region” or “immunoglobulin hinge region” herein is meant the flexible polypeptide comprising the amino acids between the first and second heavy chain constant domains of an antibody. Structurally, the IgG CH1 domain ends at EU position 215, and the IgG CH2 domain begins at residue EU position 231. Thus for IgG the antibody hinge is herein defined to include positions 216 (E216 in IgG1) to 230 (P230 in IgG1), wherein the numbering is according to the EU index as in Kabat. In some embodiments, for example in the context of an Fc region, the hinge (full length or a fragment of the hinge) is included, generally referring to positions 216-230. As noted herein, pI variants can be made in the hinge region as well.

The subject HILBAs described herein include two binding domains, including at least one scFv (see (scFv-scCLCH)₁ format). In some embodiments, the HILBA also includes a Fab binding domain. Such binding domains of the subject HILBA are described in greater below.

### A. Antibody Formats

The HILBAs described herein include two different formats, termed “(scFv-scCLCH)₁” and “(scFv-scCLCH)₂” Embodiments of the “(scFv-scCLCH)₁” and “(scFv-scCLCH)₂” are shown in FIGS. 12A and 12B, respectively. Each of these formats include at least one monomer that includes, from N- to C-terminus, a VH-scFv linker-VL-CL-domain linker-CH1-hinge-CH2-CH3. The VH-scFv linker-VL forms a single chain variable fragment (scFv) and the CL-domain linker-CH1-CH2-CH3 unit is referred to herein as a “single chain constant light domain/constant heavy domain” or “scCLCH”. Aspects of each of the two formats are described in detail below.

1. (scFv-scCLCH)₁

In one aspect, provided herein are heterodimeric proteins that include one monomer that that includes, from N- to C-terminus, a VH-scFv linker-VL-CL-domain linker-CH1-hinge-CH2-CH3. In some embodiments, the heterodimeric protein is a HILBA that has a structure according to (scFv-scCLCH)₁, as shown in FIG. 12A. This format includes a first monomer that includes, from N- to C-terminus: VH1-CH1-hinge-CH2-CH3, wherein VH1 is a first variable heavy domain and CH2-CH3 is a first Fc domain; b) a second monomer that includes, from N- to C-terminus: VL1-CL, wherein VL1 is a first variable light domain; and c) a third monomer that includes, from N- to C-terminus: scFv-CL-domain linker-CH1-hinge-CH2-CH3, wherein CH2-CH3 is a second Fc domain. In such an embodiment, VH1 and VL1 form an antigen binding domain that binds a first antigen and scFv binds a second antigen that is different from the first antigen.

In some embodiments, the scFv includes, from N- to C-terminus, VH2-scFv linker-VL2, wherein VH2 is a second variable heavy domain and VL2 is a second variable light domain. In other embodiments, the scFv is, from N- to C-terminus VL2-scFv linker-VH2. Any scFv linker can be included in the scFv. In some embodiments, the scFv linker is an scFv linker depicted in FIG. 5 (SEQ ID NOs: 1-2, 4-23 and 247). In an exemplary embodiment, the scFv linker is (GKPGS)₄ (SEQ ID NO: 2).

The domain linker that connects the CL to the CH1 in the third monomer can be any domain linker, including, but not limited to those in FIG. 6 (SEQ ID NOs: 1-3, 11, 17, 24-39 and 247). In some embodiments, the domain linker that connects the CL to the CH1 in the third monomer is (GGGGS)₅ (SEQ ID NO: 3).

As described herein, the CL-domain linker-CH1-CH2-CH3 of the subject HILBAs provided herein is referred to as a “single chain constant light domain/constant heavy domain” or “scCLCH.” In certain embodiments, the scCLCH is according to any one of the sequences in FIG. 9. In some embodiments, the scCLCH is a sequence that is at least 90, 95, 98 or 99% identical to a sequence in FIG. 9. In one embodiment, the scCLCH includes a sequence that has 1, 2, 3, 4, 5, 6, 7, 8, 9 or 10 additional amino acid modifications as compared to any one of the sequences in FIG. 9.

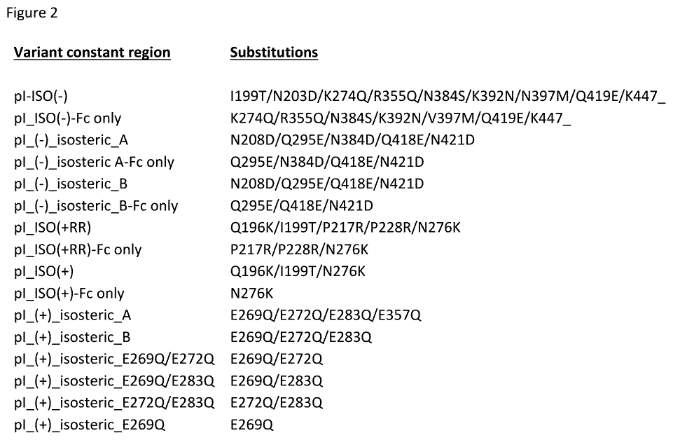

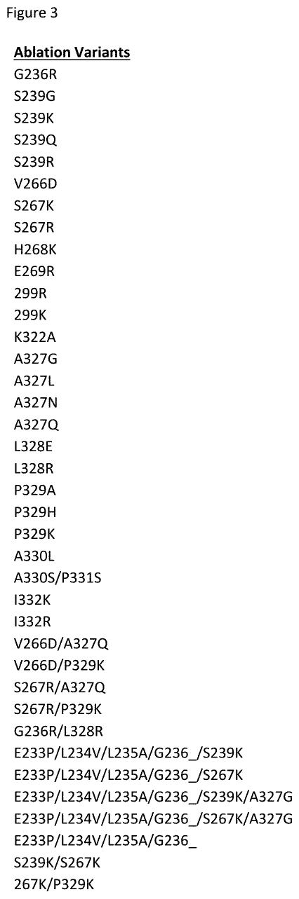

In certain embodiments, the first and second constant regions of the (scFv-scCLCH)₁ format HILBA each are variant constant regions that include one or more amino acid modifications. Particular amino acid modifications (i.e., “variants”) that can be included in the first and second Fc domains include, for example, one or more heterodimerization variant (e.g., FIG. 1), isosteric variants (e.g., FIG. 2), and/or ablation/FcKO variants (e.g., FIG. 3). Such variants that are included in particular embodiments of the (scFv-scCLCH)₁ format HILBA are discussed in detail below.

In some embodiments, the first and second Fc domains are variant Fc domains that include the heterodimerization variant set: L368D/K370S:S364K/E357Q. In such embodiments, either the first or the second variant Fc domain includes the amino acid variants L368D and K370S and the other variant Fc domain includes the amino acid variants S364K and E357Q. In certain embodiments, either the first or third monomer includes a heavy chain constant domain (CH1-hinge-CH2-CH3) with the isosteric pI substitutions N208D/Q295E/N384D/Q418E/N421D. In an exemplary embodiment, both the first and second Fc domains are variant Fc domains that each include the FcKO variants E233P/L234V/L235A/G236del/S267K.

In some embodiments, the (scFv-scCLCH)₁ constant heavy domains (i.e., CH1-hinge-CH2-CH3) of the first and third monomers have the sequences of any one of the pairs of constant heavy domain monomers in FIG. 7.

As discussed above, in some embodiments of the (scFv-scCLCH)₁ format HILBA, the third monomer is, from N- to C-terminus: VH2-scFv linker-VL2-CL-domain linker-CH1-hinge-CH2-CH3. As such, VH2 is not covalently attached to CH1 as it would be in a canonical IgG heavy chain (i.e., VH-CH1-hinge-CH2-CH3). To improve the stability of the third monomer, amino acid substitutions for cysteine residues can be engineered into the VH2 and constant chain heavy domain (i.e., CH1-hinge-CH2-CH3) of the third monomer (see FIG. 20A). In some embodiments, the engineered cysteine residue is a substitution of an amino acid in the VH and the constant chain heavy domain of the third monomer. Such cysteine residues (i.e., “dsHC variants”) allow the formation of disulfide bonds between the VH2 and the constant chain heavy domain, thereby increasing the stability of the third monomer. Any suitable amino acid position in the VH2 and the constant chain heavy domain can be used to introduce such cysteine amino acid substitutions. Preferably, these substitutions do not affect the function of these domains. For example, preferred positions for cysteine substitutions in the VH2 region are in positions that do not affect antigen binding. In exemplary embodiments, the cysteine substitutions are introduced into the C-terminus of the VH2 and the N-terminus of the constant chain heavy domain. In some embodiments, the substitution in VH2 is S112C or S113C, wherein numbering is according to Kabat numbering (see Table 1). In some embodiments, the substitution in the N-terminus of the constant chain heavy domain of the third monomer is in A118C, according to EU numbering (or A114C according to Kabat).

In addition to cysteine substitutions for increasing stability between the VH and CH1 regions of the third monomer, cysteine substitutions can also be introduced independently into VH2 and VL2 of the scFv (i.e., “dsscFv variants”) in order to form one or more disulfide bonds in these two domains, thereby increasing the stability of the scFv in the (scFv-scCLCH)₁ format HILBA (see FIG. 20B). Any suitable position in the VH2 and VL2 can be used to introduce such cysteine amino acid substitutions. Preferably, these substitutions do not affect the function of these domains. For example, preferred positions for cysteine substitutions in the VH2 and VL2 domains are in positions that do not affect antigen binding. In some embodiments, the substitution is G44C in VH2 and G100C in VL2, according to Kabat numbering (see Table 1).

In some embodiments, the third monomer includes a) a first engineered cysteine in VH2, b) a second engineered cysteine in the VH2; c) a third engineered cysteine in CH1; and d) a fourth engineered cysteine in VL2. In such embodiments, the first engineered cysteine residue and the third engineered cysteine residue form a first disulfide bond, and the second engineered cysteine residue and the fourth engineered cysteine residue form a second disulfide bond (see FIG. 20C). In an exemplary embodiment, the first engineered cysteine residue is amino acid substitution S112C or S113C according to Kabat numbering, the second engineered cysteine residue is amino acid substitution G44C according to Kabat numbering, the third engineered cysteine residue is amino acid substitution A118C according to EU numbering (or A114C according to Kabat), and the fourth engineered cysteine residue is amino acid substitution G100C according to Kabat numbering.

Exemplary HILBAs having the (scFv-scCLCH)₁ format include, for example, are shown in FIGS. 13, 22 and 30.

In some embodiments, the heterodimeric protein includes a first monomer that includes a CH2-CH3, wherein CH2-C3 is a first Fc domain; and a second monomer that includes, from N- to C-terminus, a VH-scFv linker-VL-CL-domain linker-CH1-hinge-CH2-CH3, where VH is a variable heavy domain, VL is a variable light domain, and CH2-CH3 is a second Fc domain. The VH and VL form an antigen binding domain. In some embodiments, the heterodimeric protein includes a fusion partner operably linked to the first Fc domain. Exemplary fusion partners include, but are not limited to, single peptides, ligands activated upon cell-surface receptor binding, signaling molecules, cytokines, extracellular domains of a receptors and bait proteins used to identify binding partners in protein microarrays.

In such embodiments, VH is not covalently attached to CH1 as it would be in a canonical IgG heavy chain (i.e., VH-CH1-hinge-CH2-CH3). To improve the stability of the second monomer, amino acid substitutions for cysteine residues can be engineered into the VH and constant chain heavy domain (i.e., CH1-hinge-CH2-CH3) of the second monomer. In some embodiments, the engineered cysteine residue is a substitution of an amino acid in the VH and the constant chain heavy domain of the second monomer. Such cysteine residues (i.e., “dsHC variants”) allow the formation of disulfide bonds between the VH and the constant chain heavy domain, thereby increasing the stability of the second monomer. Any suitable amino acid position in the VH and the constant chain heavy domain can be used to introduce such cysteine amino acid substitutions. Preferably, these substitutions do not affect the function of these domains. For example, preferred positions for cysteine substitutions in the VH region are in positions that do not affect antigen binding. In exemplary embodiments, the cysteine substitutions are introduced into the C-terminus of the VH and the N-terminus of the constant chain heavy domain. In some embodiments, the substitution in VH is S112C or S113C, wherein numbering is according to Kabat numbering (see Table 1). In some embodiments, the substitution in the N-terminus of the constant chain heavy domain of the second monomer is in A118C, according to EU numbering (or A114C according to Kabat).

In addition to cysteine substitutions for increasing stability between the VH and CH1 regions of the second monomer, cysteine substitutions can also be introduced independently into VH and VL of the scFv (i.e., “dsscFv variants”) in order to form one or more disulfide bonds in these two domains, thereby increasing the stability of the scFv in the (scFv-scCLCH)₁ format HILBA (see FIG. 20B). Any suitable position in the VH and VL can be used to introduce such cysteine amino acid substitutions. Preferably, these substitutions do not affect the function of these domains. For example, preferred positions for cysteine substitutions in the VH and VL domains are in positions that do not affect antigen binding. In some embodiments, the substitution is G44C in VH2 and G100C in VL2, according to Kabat numbering (see Table 1).

In some embodiments, the second monomer includes a) a first engineered cysteine in VH, b) a second engineered cysteine in the VH; c) a third engineered cysteine in CH1; and d) a fourth engineered cysteine in VL2. In such embodiments, the first engineered cysteine residue and the third engineered cysteine residue form a first disulfide bond, and the second engineered cysteine residue and the fourth engineered cysteine residue form a second disulfide bond. In an exemplary embodiment, the first engineered cysteine residue is amino acid substitution S112C or S113C according to Kabat numbering, the second engineered cysteine residue is amino acid substitution G44C according to Kabat numbering, the third engineered cysteine residue is amino acid substitution A118C according to EU numbering (or A114C according to Kabat), and the fourth engineered cysteine residue is amino acid substitution G100C according to Kabat numbering.

2. (scFv-scCLCH)₂

In some embodiments, the HILBA has a structure according tine (scFv-scCLCH)₂, as shown in FIG. 12B. This format includes a first monomer that includes, from N- to C-terminus: scFv1-CL-domain linker-CH1-hinge-CH2-CH3, wherein CH2-CH3 is a first Fc domain; and b) a second monomer that includes, from N- to C-terminus: scFv2-CL-domain linker-CH1-hinge-CH2-CH3, wherein CH2-CH3 is a second Fc domain. In the (scFv-scCLCH)₂ format, scFv1 and scFv2 each bind a different antigen.

In some embodiments, scFv1 includes, from N- to C-terminus: VH1-scFv linker-VL1, and scFv2 is from N- to C-terminus: VH2-scFv linker-VL2. In other embodiments, scFv1 includes, from N- to C-terminus: VL1-scFv linker-VH1, and scFv2 is from N- to C-terminus: VL2-scFv linker-VH2. Any scFv linker can be included in the scFv. In some embodiments, the scFv linker is an scFv linker depicted in FIG. 5 (SEQ ID NOs: 1-2, 4-23 and 247). In an exemplary embodiment, the scFv linker is (GKPGS)₄ (SEQ ID NO: 2).

The domain linker that connects the CL to the CH1 in each of the first and second monomers can be any domain linker, including, but not limited to those in FIG. 6 (SEQ ID NOs: 1-3, 11, 17, 24-39 and 247). In some embodiments, the domain linker that connects the CL to the CH1 in each of the first and second monomers is (GGGGS)₅ (SEQ ID NO: 3).

As described herein, the CL-domain linker-CH1-hinge-CH2-CH3 of the subject HILBAs is referred to as a “single chain constant light domain/constant heavy domain” or “scCLCH.” In the (scFv-scCLCH)₂ format, each of the first and second monomers include a scCLCH. In certain embodiments, the scCLCHs are according to any one of the sequences in FIG. 9. In some embodiments, the scCLCHs of each of the first and second monomers has a sequence that is at least 90, 95, 98 or 99% identical to a sequence in FIG. 9. In one embodiment, each of the scCLCHs of the first and second monomer includes a sequence that has 1, 2, 3, 4, 5, 6, 7, 8, 9 or 10 additional amino acid modifications as compared to any one of the sequences in FIG. 9.

In certain embodiments, the first and second constant domains of the (scFv-scCLCH)₂ format HILBA each are variant constant domains that include one or more amino acid modifications. Particular amino acid modifications (i.e., “variants”) that can be included in the first and second Fc domains include, for example, one or more heterodimerization variant (e.g., FIG. 1), isosteric variants (e.g., FIG. 2), and/or ablation/FcKO variants (e.g., FIG. 3). Such variants that are included in particular embodiments of the (scFv-scCLCH)₁ format HILBA are discussed in detail below.

In some embodiments, the first and second Fc domains are variant Fc domains that include the heterodimerization variant set: L368D/K370S:S364K/E357Q. In such embodiments, either the first or the second variant Fc domain includes the amino acid variants L368D and K370S and the other variant Fc domain includes the amino acid variants S364K and E357Q. In certain embodiments, either the first or second monomer includes a heavy chain constant domain (CH1-hinge-CH2-CH3) with the isosteric pI substitutions N208D/Q295E/N384D/Q418E/N421D. In an exemplary embodiment, both the first and second Fc domains are variant Fc domains that each include the FcKO variants E233P/L234V/L235A/G236del/S267K.

In some embodiments, the (scFv-scCLCH)₂ constant heavy domains (i.e., CH1-hinge-CH2-CH3) of the first and second monomers have the sequences of any one of the pairs of constant heavy domain monomers in FIG. 7.

In some embodiments, each of the first of the second monomers include engineered cysteine residues that form intra-disulfide bonds between the VH and the corresponding constant chain heavy domain of each monomer, thereby stabilizing the respective monomer (“dsHC variants,” see FIG. 20D). In some embodiments, the engineered cysteine residue is an amino acid substitution of an amino acid in the VH and the corresponding constant chain heavy domain of the first and/or second monomer. Any suitable amino acid substitution in the VH1/VH2 and its respective constant chain heavy domain can be used to introduce such cysteine amino acid substitutions. Preferably, these substitutions do not affect the function of these domains. For example, preferred positions for cysteine substitutions in the VH1/VH2 domains are in positions that do not affect antigen binding. In exemplary embodiments, the cysteine substitutions are introduced into the C-terminus of the VH1/VH2 and the N-terminus of the corresponding constant chain heavy domain. In some embodiments, the substitution in VH1 and VH2 is S112C or S113C, wherein numbering is according to Kabat numbering (see Table 1). In some embodiments, the substitution in the N-terminus of the constant chain heavy domain of each of the first and second monomers is A118C, according to EU numbering (or A114C according to Kabat).

In addition to cysteine substitutions for increasing stability between the VH1/VH2 and corresponding CH1 regions of the first and second monomers, cysteine substitutions can also be introduced independently into VH1 and VL1 of scFv1 and VH2 and VL2 of scFv2 in order to form one or more intra-disulfide bonds in these two domains, thereby increasing the stability of each of the scFvs (“dsscFv variants,” see FIG. 20E). Any suitable position in the VH1/VH2 and VL1/VL2 domains can be used to introduce such cysteine amino acid substitutions. Preferably, these substitutions do not affect the function of these domains. For example, preferred positions for cysteine substitutions in the VH and VL domains are in positions that do not affect antigen binding. In some embodiments, the substitution is G44C in VH1 and VH2 and G100C in VL1 and VL2, according to Kabat numbering (see Table 1).

In some embodiments, the (scFv-scCLCH)₂ format HILBA includes a) a first engineered cysteine in VH1, b) a second engineered cysteine in the VH1; c) a third engineered cysteine in CH1 of the first monomer; d) a fourth engineered cysteine residue in VL1; e) a fifth engineered cysteine residue in VH2; f) a sixth engineered cysteine residue in VH2; g) a seventh engineered cysteine residue in CH1 of the second monomer; and h) an eight engineered cysteine residue in VL2. (see FIG. 20F). In such an embodiment, the first engineered cysteine residue and the third engineered cysteine residue form a first disulfide bond, the second engineered cysteine residue and the fourth engineered cysteine residue form a second disulfide bond, the fifth engineered cysteine residue and the seventh engineered cysteine residue from a third disulfide bond, and the sixth engineered cysteine residue and eighth engineered cysteine residue from a fourth disulfide bond.

In an exemplary embodiment, the first and fifth engineered cysteine residues are amino acid substitution S112C or S113C according to Kabat numbering, the second and sixth engineered cysteine residues are each amino acid substitution G44C according to Kabat numbering, the third and seventh engineered cysteine residues are each amino acid substitution A118C according to EU numbering (or A114C according to Kabat), and the fourth and eight engineered cysteine residues are each amino acid substitution G100C according to Kabat numbering.

3. Engineered Disulfide Bonds

As discussed herein the subject HILBAs provided herein include at least one monomer that includes, from N- to C-terminus: VH-scFv linker-VL-CL-domain linker-CH1-hinge-CH2-CH3. Thus, the VH domain of such a monomer is not covalently attached to CH1 as it would be in a canonical IgG heavy chain (i.e., VH-CH1-hinge-CH2-CH3). To improve the stability of such a monomer, amino acid substitutions for cysteine residues can be engineered into the VH and heavy chain constant domain (i.e., CH1-hinge-CH2-CH3). HILBAs that include such disulfide bonds are referred to as “dsHC” and the engineered cysteine residues in the VH and heavy chain constant domain are referred to as “dsHC variants.” In some embodiments, the engineered cysteine residue is an amino acid substitution of an amino acid in the VH and the constant chain heavy domain of such a monomer. Such cysteine residues allow the formation of disulfide bonds between the VH2 and the constant chain heavy domain, thereby increasing the stability of the monomer. Any suitable amino acid position in the VH and the constant chain heavy domain can be used to introduce such cysteine amino acid substitutions. Preferably, these substitutions do not affect the function of these domains. For example, preferred positions for cysteine substitutions in the VH region are in positions that do not affect antigen binding. In exemplary embodiments, the cysteine substitutions are introduced into the C-terminus of the VH2 and the N-terminus of the constant chain heavy domain. In some embodiments, the substitution in VH is S112C or S113C, wherein numbering is according to Kabat numbering (see Table 1). In some embodiments, the substitution in the N-terminus of the constant chain heavy domain is A118C, according to EU numbering (or A114C according to Kabat).

In addition to cysteine substitutions for increasing stability between the VH and CH regions of such a monomer, cysteine substitutions can also be introduced independently into VH and VL of the scFv in order to form one or more disulfide bonds in these two domains, thereby increasing the stability of the scFv. HILBAs that include such disulfide bonds are referred to as “dsscFv” and the engineered cysteine residues in the VH and heavy chain constant domain are referred to as “dsscFv variants.” Any suitable position in the VH2 and VL2 can be used to introduce such cysteine amino acid substitutions. Preferably, these substitutions do not affect the function of these domains. For example, preferred positions for cysteine substitutions in the VH2 and VL2 domains are in positions that do not affect antigen binding. In some embodiments, the substitution is G44C in VH2 and G100C in VL2, according to Kabat numbering (see Table 1).

In some embodiments, the HILBA includes dsscFv variants. In certain embodiments, the HILBA includes dsHC variants. In exemplary embodiments, the HILBA includes both dsscFv and dsHC variants.

### B. Heterodimerization Variants

In some the heterodimeric Ig-G like bispecific antibody (HILBA) includes modifications that facilitate the heterodimerization of the first and second monomers and/or allow for ease of purification of heterodimers over homodimers, collectively referred to herein as “heterodimerization variants.” As discussed below, heterodimerization variants can include, for example, skew variants (e.g., the “knobs and holes” and “charge pairs” variants described below) as well as “pI variants” that facilitates the separation of homodimers away from heterodimers, as well as other Fc variants discussed herein. As is generally described in U.S. Pat. No. 9,605,084, hereby incorporated by reference in its entirety and specifically as below for the discussion of heterodimerization variants, useful mechanisms for heterodimerization include “knobs and holes” (“KIH”) as described in U.S. Pat. No. 9,605,084, “electrostatic steering” or “charge pairs” as described in U.S. Pat. No. 9,605,084, pI variants as described in U.S. Pat. No. 9,605,084, and general additional Fc variants as outlined in U.S. Pat. No. 9,605,084 and below.

1. Skew Variant

In some embodiments, the heterodimeric Ig-G like bispecific antibodies (HILBA) and heterodimeric proteins provided herein include skew variants, which are one or more amino acid modifications in a first Fc domain (A) and/or a second Fc domain (B) that favor the formation of Fc heterodimers (Fc dimers that include the first and the second Fc domain; A-B) over Fc homodimers (Fc dimers that include two of the first Fc domain or two of the second Fc domain; A-A or B-B). Suitable skew variants are included in the FIG. 29 of US Publ. App. No. 2016/0355608, hereby incorporated by reference in its entirety and specifically for its disclosure of skew variants, as well as in FIG. 1.

One mechanism for skew variants is generally referred to in the art as “knobs and holes,” referring to amino acid engineering that creates steric influences to favor heterodimeric formation and disfavor homodimeric formation, as described in U.S. Ser. No. 61/596,846, Ridgway et al., Protein Engineering 9(7):617 (1996); Atwell et al., J. Mol. Biol. 1997 270:26; U.S. Pat. No. 8,216,805, all of which are hereby incorporated by reference in their entirety and specifically for the disclosure of “knobs and holes” mutations. This is sometime referred to herein as “steric variants.” The figures identify a number of “monomer A-monomer B” pairs that rely on “knobs and holes”. In addition, as described in Merchant et al., Nature Biotech. 16:677 (1998), these “knobs and holes” mutations can be combined with disulfide bonds to further favor formation of Fc heterodimers.

An additional mechanism for skew variants that finds use in the generation of heterodimers is sometimes referred to as “electrostatic steering” as described in Gunasekaran et al., J. Biol. Chem. 285(25):19637 (2010), hereby incorporated by reference in its entirety. This is sometimes referred to herein as “charge pairs.” In this embodiment, electrostatics are used to skew the formation towards heterodimerization. As those in the art will appreciate, these may also have an effect on pI, and thus on purification, and thus could in some cases also be considered pI variants. However, as these were generated to force heterodimerization and were not used as purification tools, they are classified as “skew variants”. These include, but are not limited to, D221E/P228E/L368E paired with D221R/P228R/K409R (e.g., these are “monomer” corresponding sets) and C220E/P228E/368E paired with C220R/E224R/P228R/K409R.

In some embodiments, the skew variants advantageously and simultaneously favor heterodimerization based on both the “knobs and holes” mechanism as well as the “electrostatic steering” mechanism. In some embodiments, the heterodimeric Ig-G like bispecific antibody (HILBA) includes one or more sets of such heterodimerization skew variants. Exemplary skew variants that fall into this category include: S364K/E357Q:L368D/K370S; L368D/K370S:S364K; L368E/K370S:S364K; T411T/E360E/Q362E:D401K; L368D/K370S:S364K/E357L; K370S:S364K/E357Q; or a T366S/L368A/Y407V:T366W (optionally including a bridging disulfide, T366S/L368A/Y407V/Y349C:T366W/S354C). These variants come in “pairs” of “sets.” That is, one set of the pair is incorporated into the first monomer and the other set of the pair is incorporated into the second monomer. In terms of nomenclature, the pair “S364K/E357Q:L368D/K370S” means that one of the monomers includes an Fc domain that includes the amino acid substitutions S364K and E357Q and the other monomer includes an Fc domain that includes the amino acid substitutions L368D and K370S; as above, the “strandedness” of these pairs depends on the starting pI. It should be noted that these sets do not necessarily behave as “knobs in holes” variants, with a one-to-one correspondence between a residue on one monomer and a residue on the other. That is, these pairs of sets may instead form an interface between the two monomers that encourages heterodimer formation and discourages homodimer formation, allowing the percentage of heterodimers that spontaneously form under biological conditions to be over 90%, rather than the expected 50% (25% homodimer A/A:50% heterodimer A/B:25% homodimer B/B). Exemplary heterodimerization “skew” variants are depicted in FIG. 1.

In exemplary embodiments, the heterodimeric Ig-G like bispecific antibody (HILBA) includes a S364K/E357Q:L368D/K370S; L368D/K370S:S364K; L368E/K370S:S364K; T411T/E360E/Q362E:D401K; L368D/K370S:S364K/E357L; K370S:S364K/E357Q; or a T366S/L368A/Y407V:T366W (optionally including a bridging disulfide, T366S/L368A/Y407V/Y349C:T366W/S354C) “skew” variant amino acid substitution set. In an exemplary embodiment, the HILBA includes first and second variant Fc domains with a “S364K/E357Q:L368D/K370S” amino acid substitution set.

In some embodiments, the skew variants provided herein can be optionally and independently incorporated with any other modifications, including, but not limited to, other skew variants (see, e.g., in FIG. 37 of US Publ. App. No. 2012/0149876, herein incorporated by reference, particularly for its disclosure of skew variants), pI variants, isotypic variants, FcRn variants, ablation variants, etc. into one or both of the first and second Fc domains of the heterodimeric Ig-G like bispecific antibody. Further, individual modifications can also independently and optionally be included or excluded from the subject heterodimeric Ig-G like bispecific antibodies.

2. pI (Isoelectric Point) Variants for Heterodimers

In some embodiments, the heterodimeric Ig-G like bispecific antibodies (HILBA) and heterodimeric proteins provided herein include purification variants that advantageously allow for the separation of HILBAs from homodimeric proteins.

There are several basic mechanisms that can lead to ease of purifying heterodimeric proteins. One such mechanism relies on the use of pI variants which include one or more modifications that affect the isoelectric point of one or both of the monomers of the fusion protein, such that each monomer, and subsequently each dimeric species, has a different pI, thus allowing the isoelectric purification of A-A, A-B and B-B dimeric proteins. Alternatively, some formats also allow separation on the basis of size. As is further outlined below, it is also possible to “skew” the formation of heterodimers over homodimers using skew variants. Thus, a combination of heterodimerization skew variants and pI variants find particular use in the subject heterodimeric Ig-G like bispecific antibodies provided herein.

Additionally, as more fully outlined below, depending on the format of the heterodimeric Fc fusion protein, pI variants can be either contained within the constant region and/or Fc domains of a monomer, and/or domain linkers can be used. In some embodiments, the heterodimeric Ig-G like bispecific antibody includes additional modifications for alternative functionalities can also create pI changes, such as Fc, FcRn and KO variants.

In the embodiments that utilizes pI as a separation mechanism to allow the purification of heterodimeric Ig-G like bispecific antibodies, amino acid modifications can be introduced into one or both of the constant domains (i.e., CH1-hinge-CH2-CH3). That is, the pI of one of the monomers (referred to herein for simplicity as “monomer A”) can be engineered away from monomer B, or both monomer A and B can be changed, with the pI of monomer A increasing and the pI of monomer B decreasing. As discussed, the pI changes of either or both monomers can be done by removing or adding a charged residue (e.g., a neutral amino acid is replaced by a positively or negatively charged amino acid residue, e.g., glutamine to glutamic acid), changing a charged residue from positive or negative to the opposite charge (e.g. aspartic acid to lysine) or changing a charged residue to a neutral residue (e.g., loss of a charge; lysine to serine.). A number of these variants are shown in the figures, including, FIGS. 4 and 5.

Creating a sufficient change in pI in at least one of the monomers such that heterodimers can be separated from homodimers can be done by using a “wild type” heavy chain constant region and a variant region that has been engineered to either increase or decrease its pI (wt A:B+ or wt A:B−), or by increasing one region and decreasing the other region (A+:B− or A−:B+).

Thus, in general, a component of some embodiments of the present subject fusion proteins are amino acid variants in the Fc domains or constant domain regions that are directed to altering the isoelectric point (pI) of at least one, if not both, of the monomers of a dimeric protein by incorporating amino acid substitutions (“pI variants” or “pI substitutions”) into one or both of the monomers. The separation of the heterodimers from the two homodimers can be accomplished if the pIs of the two monomers differ by as little as 0.1 pH unit, with 0.2, 0.3, 0.4 and 0.5 or greater all finding use in the present invention.

As will be appreciated by those in the art, the number of pI variants to be included on each or both constant domains (i.e., CH1-hinge-CH2-CH3) of a HILBA to achieve good separation will depend in part on the starting pI of the components. That is, to determine which monomer to engineer or in which “direction” (e.g., more positive or more negative), the sequences of the Fc domains and any other domains and/or domain linkers (e.g., scFv, scFv linkers, domain linkers, Fabs, etc.) in each monomer are calculated and a decision is made from there based on the pIs of the monomers. As is known in the art, different Fc domains, linkers, and binding domains will have different starting pIs. In general, as outlined herein, the pIs are engineered to result in a total pI difference of each monomer of at least about 0.1 logs, with 0.2 to 0.5 being preferred as outlined herein.

In general, as will be appreciated by those in the art, there are two general categories of amino acid modifications that affect pI: those that increase the pI of the protein (basic changes) and those that decrease the pI of the protein (acidic changes). As described herein, all combinations of these variants can be used: one monomer may include a wild type Fc domain, or a variant Fc domain that does not display a significantly different pI from wild-type, and the other monomer includes a Fc domain that is either more basic or more acidic. Alternatively, each monomer may be changed, one to more basic and one to more acidic.

In the case where pI variants are used to achieve heterodimerization, a more modular approach to designing and purifying HILBAs is provided. Thus, in some embodiments, heterodimerization variants (including skew and pI variants) must be engineered. In addition, in some embodiments, the possibility of immunogenicity resulting from the pI variants is significantly reduced by importing pI variants from different IgG isotypes such that pI is changed without introducing significant immunogenicity (see isotypic variants below). Thus, an additional problem to be solved is the elucidation of low pI constant domains with high human sequence content, e.g. the minimization or avoidance of non-human residues at any particular position. Alternatively or in addition to isotypic substitutions, the possibility of immunogenicity resulting from the pI variants is significantly reduced by utilizing isosteric substitutions (e.g. Asn to Asp; and Gln to Glu).

A side benefit that can occur with this pI engineering is also the extension of serum half-life and increased FcRn binding. That is, as described in US Publ. App. No. US 2012/0028304 (incorporated by reference in its entirety and specifically for the disclosure of pI variants that provide additional function), lowering the pI of antibody constant domains (including those found in Fc fusions) can lead to longer serum retention in vivo. These pI variants for increased serum half-life also facilitate pI changes for purification.

In addition, it should be noted that the pI variants of the heterodimerization variants give an additional benefit for the analytics and quality control process of Fc fusion proteins, as the ability to either eliminate, minimize and distinguish when homodimers are present is significant. Similarly, the ability to reliably test the reproducibility of the heterodimeric Fc fusion protein production is important.

Exemplary combinations of pI variants are shown in FIGS. 2 and 3, and FIG. 30 of US Publ. App. No. 2016/0355608, all of which are herein incorporated by reference in its entirety and specifically for the disclosure of pI variants. As outlined herein and shown in the figures, these changes are shown relative to IgG1, but all isotypes can be altered this way, as well as isotype hybrids. In the case where the heavy chain constant domain is from IgG2-4, R133E and R133Q can also be used.

In one embodiment, the heterodimeric IgG-like bispecific antibody includes a monomer with a variant constant domain having pI variant modifications N208D/Q295E/N384D/Q418E/N421D. In one embodiment, the heterodimeric IgG-like bispecific antibody includes a monomer with a variant Fc domain having pI variant modifications 295E/384D/418E/421D (Q295E/N384D/Q418E/N421D when relative to human IgG1). In one embodiment, the heterodimeric IgG-like bispecific antibody includes a monomer with a variant Fc domain having pI variant modifications 217R/228R/276K (P217R/P228R/N276K when relative to human IgG1). Additional exemplary pI variant modification that can be incorporated into the Fc domain of a subject are depicted in FIG. 2.

In some embodiments, modifications are made in the hinge of the Fc domain, including positions 216, 217, 218, 219, 220, 221, 222, 223, 224, 225, 226, 227, 228, 229, and 230 based on EU numbering. Thus, pI mutations and particularly substitutions can be made in one or more of positions 216-230, with 1, 2, 3, 4 or 5 mutations finding use. Again, all possible combinations are contemplated, alone or with other pI variants in other domains.

Specific substitutions that find use in lowering the pI of hinge domains include, but are not limited to, a deletion at position 221, a non-native valine or threonine at position 222, a deletion at position 223, a non-native glutamic acid at position 224, a deletion at position 225, a deletion at position 235 and a deletion or a non-native alanine at position 236. In some cases, only pI substitutions are done in the hinge domain, and in others, these substitution(s) are added to other pI variants in other domains in any combination.

In some embodiments, mutations can be made in the CH2 region, including positions 233, 234, 235, 236, 274, 296, 300, 309, 320, 322, 326, 327, 334 and 339, based on EU numbering. It should be noted that changes in 233-236 can be made to increase effector function (along with 327A) in the IgG2 backbone. Again, all possible combinations of these 14 positions can be made; e.g., a HILBA may include a variant Fc domain with 1, 2, 3, 4, 5, 6, 7, 8, 9 or 10 CH2 pI substitutions.

Specific substitutions that find use in lowering the pI of CH2 domains include, but are not limited to, a non-native glutamine or glutamic acid at position 274, a non-native phenylalanine at position 296, a non-native phenylalanine at position 300, a non-native valine at position 309, a non-native glutamic acid at position 320, a non-native glutamic acid at position 322, a non-native glutamic acid at position 326, a non-native glycine at position 327, a non-native glutamic acid at position 334, a non-native threonine at position 339, and all possible combinations within CH2 and with other domains.

In this embodiment, the modifications can be independently and optionally selected from position 355, 359, 362, 384, 389, 392, 397, 418, 419, 444 and 447 (EU numbering) of the CH3 region. Specific substitutions that find use in lowering the pI of CH3 domains include, but are not limited to, a non-native glutamine or glutamic acid at position 355, a non-native serine at position 384, a non-native asparagine or glutamic acid at position 392, a non-native methionine at position 397, a non-native glutamic acid at position 419, a non-native glutamic acid at position 359, a non-native glutamic acid at position 362, a non-native glutamic acid at position 389, a non-native glutamic acid at position 418, a non-native glutamic acid at position 444, and a deletion or non-native aspartic acid at position 447.

3. Isotypic Variants

In addition, some embodiments of the heterodimeric Ig-G like bispecific antibodies (HILBA) and heterodimeric proteins provided herein rely on the “importation” of pI amino acids at particular positions from one IgG isotype into another, thus reducing or eliminating the possibility of unwanted immunogenicity being introduced into the variants. A number of these are shown in FIG. 21 of US Publ. App. No. 2014/0370013, hereby incorporated by reference, particularly for its disclosure of isotypic variants. That is, IgG1 is a common isotype for therapeutic antibodies for a variety of reasons, including high effector function. However, the heavy constant region of IgG1 has a higher pI than that of IgG2 (8.10 versus 7.31). By introducing IgG2 residues at particular positions into the IgG1 backbone, the pI of the resulting monomer is lowered (or increased) and additionally exhibits longer serum half-life. For example, IgG1 has a glycine (pI 5.97) at position 137, and IgG2 has a glutamic acid (pI 3.22); importing the glutamic acid will affect the pI of the resulting protein. As is described below, a number of amino acid substitutions are generally required to significantly affect the pI of the variant Fc fusion protein. However, it should be noted as discussed below that even changes in IgG2 molecules allow for increased serum half-life.

In other embodiments, non-isotypic amino acid modifications are made, either to reduce the overall charge state of the resulting protein (e.g., by changing a higher pI amino acid to a lower pI amino acid), or to allow accommodations in structure for stability, etc. as is further described below.

In addition, by pI engineering both the heavy and light constant domains, significant modifications in each monomer of the heterodimer can be seen. As discussed herein, having the pIs of the two monomers differ by at least 0.5 can allow separation by ion exchange chromatography or isoelectric focusing, or other methods sensitive to isoelectric point.

4. Calculating pI

The pI of each monomer of the heterodimeric IgG-like bispecific antibody can depend on the pI of the variant Fc domain and the pI of the total monomer, including the scFvs, constant domains, variable domains and/or domain linker included in each monomer. Thus, in some embodiments, the change in pI is calculated on the basis of the variant Fc domain, using the chart in the FIG. 19 of US Publ. App. No. 2014/0370013, hereby incorporated by reference, particularly for its disclosure of methods of calculating pI. As discussed herein, which monomer to engineer is generally decided by the inherent pI of each monomer.

5. pI Variants that Also Confer Better FcRn In Vivo Binding

In the case where the pI variant(s) decreases the pI of the monomer, such modifications can have the added benefit of improving serum retention in vivo.

Fc regions are believed to have longer half-lives in vivo, because binding to FcRn at pH 6 in an endosome sequesters the Fc (Ghetie and Ward, 1997 Immunol Today. 18(12): 592-598, entirely incorporated by reference). The endosomal compartment then recycles the Fc to the cell surface. Once the compartment opens to the extracellular space, the higher pH, ˜7.4, induces the release of Fc back into the blood. In mice, Dall'Acqua et al. showed that Fc mutants with increased FcRn binding at pH 6 and pH 7.4 actually had reduced serum concentrations and the same half-life as wild-type Fc (Dall'Acqua et al. 2002, J. Immunol. 169:5171-5180, entirely incorporated by reference). The increased affinity of Fc for FcRn at pH 7.4 is thought to forbid the release of the Fc back into the blood. Therefore, the Fc modifications that will increase Fc's half-life in vivo will ideally increase FcRn binding at the lower pH while still allowing release of Fc at higher pH. The amino acid histidine changes its charge state in the pH range of 6.0 to 7.4. Thus, it is not surprising to find His residues at important positions in the Fc/FcRn complex.

### C. Other Fc Variants for Additional Functionality

In addition to heterodimerization variants, the subject heterodimeric IgG-like bispecific antibodies provided herein may independently include Fc modifications that affect functionality including, but not limited to, altering binding to one or more Fc receptors (e.g., FcγR and FcRn).

1. FcγR Variants

In one embodiment, the heterodimeric IgG-like bispecific antibody includes one or more amino acid modifications that affect binding to one or more Fcγ receptors (i.e., “FcγR variants”). FcγR variants (e.g., amino acid substitutions) that result in increased binding as well as decreased binding can be useful. For example, it is known that increased binding to FcγRIIIa results in increased ADCC (antibody dependent cell-mediated cytotoxicity; the cell-mediated reaction wherein nonspecific cytotoxic cells that express FcγRs recognize bound antibody on a target cell and subsequently cause lysis of the target cell). Similarly, decreased binding to FcγRIIb (an inhibitory receptor) can be beneficial as well in some circumstances. FcγR variants that find use in the heterodimeric IgG-like bispecific antibody include those listed in U.S. Pat. No. 8,188,321 (particularly FIGS. 41) and U.S. Pat. No. 8,084,582, and US Publ. App. Nos. 20060235208 and 20070148170, all of which are expressly incorporated herein by reference in their entirety and specifically for the variants disclosed therein that affect Fcγ receptor binding. Particular variants that find use include, but are not limited to, 236A, 239D, 239E, 332E, 332D, 239D/332E, 267D, 267E, 328F, 267E/328F, 236A/332E, 239D/332E/330Y, 239D, 332E/330L, 243A, 243L, 264A, 264V and 299T.

In addition, amino acid substitutions that increase affinity for FcγRIIc can also be independently included in the Fc domain variants outlined herein. Useful substitutions that for FcγRIIc are described in, for example, U.S. Pat. Nos. 8,188,321 and 10,113,001, all of which are expressly incorporated herein by reference in their entirety and specifically for the variants disclosed therein that affect Fcγ receptor binding.

2. FcRn Variants

Further, the heterodimeric Ig-G like bispecific antibodies (HILBA) and heterodimeric proteins provided herein can independently include Fc substitutions that confer increased binding to the FcRn and increased serum half-life. Such modifications are disclosed, for example, in U.S. Pat. No. 8,367,805, hereby incorporated by reference in its entirety, and specifically for Fc substitutions that increase binding to FcRn and increase half-life. Such modifications include, but are not limited to 434S, 434A, 428L, 308F, 2591, 428L/434S, 259I/308F, 436I/428L, 436I or V/434S, 436V/428L and 259I/308F/428L.

3. Ablation Variants

In some embodiments, the heterodimeric Ig-G like bispecific antibodies (HILBA) and heterodimeric proteins provided herein include one or more modifications that reduce or remove the normal binding of the Fc domain to one or more or all of the Fcγ receptors (e.g., FcγR1, FcγRIIa, FcγRIIb, FcγRIIIa, etc.) to avoid additional mechanisms of action. Such modifications are referred to as “FcγR ablation variants” or “Fc knock out (FcKO or KO)” variants. In some embodiments, particularly in the use of immunomodulatory proteins, it is desirable to ablate FcγRIIIa binding to eliminate or significantly reduce ADCC activity such that one of the Fc domains comprises one or more Fcγ receptor ablation variants. These ablation variants are depicted in FIG. 31 of U.S. Pat. No. 10,259,887, which is herein incorporated by reference in its entirety, and each can be independently and optionally included or excluded, with preferred aspects utilizing ablation variants selected from the group consisting of G236R/L328R, E233P/L234V/L235A/G236del/S239K, E233P/L234V/L235A/G236del/S267K, E233P/L234V/L235A/G236del/S239K/A327G, E233P/L234V/L235A/G236del/S267K/A327G and E233P/L234V/L235A/G236del, according to the EU index. In addition, ablation variants of use in the subject HILBAs are also depicted in FIG. 4. It should be noted that the ablation variants referenced herein ablate FcγR binding but generally not FcRn binding.

### D. Combination of Heterodimeric and Fc Variants

As will be appreciated by those in the art, the Fc modifications described herein can independently be combined. For example, all of the recited heterodimerization variants (including skew and/or pI variants) can be optionally and independently combined in any way, as long as they retain their “strandedness” or “monomer partition.”

In the case of pI variants, while embodiments finding particular use are shown in the figures, other combinations can be generated, following the basic rule of altering the pI difference between two monomers to facilitate purification.

In addition, any of the heterodimerization variants, may also be independently and optionally combined with other variants described herein including, but not limited to, Fc ablation variants, FcRn variants, and/or half/life extension variants as generally outlined herein.

Exemplary combinations of modifications are shown in FIG. 4 and the two heavy chain constant domain (i.e., CH1-hinge-CH2-CH3) sequences in FIG. 7. In certain embodiments, the heterodimeric IgG-like bispecific antibody includes a combination of Fc domain modifications as depicted in FIG. 4. In some embodiments, the heterodimeric IgG-like bispecific antibody includes two heavy chain constant domains (i.e., CH1-hinge-CH2-CH3) having the sequences of any pair of “constant heavy domain monomer” backbones in FIG. 7.

### E. Binding Domains

The subject heterodimeric IgG-like bispecific antibodies (HILBAs) provided herein include includes at least one antigen binding domain. In some embodiments, the subject heterodimeric IgG-like bispecific antibodies (HILBAs) provided herein include two binding domains that each bind to a different antigen. In the (scFv-scCLCH)₁ format of the subject HILBA, the two antigen binding domains are a Fab domain and an scFv domain. In the (scFv-scCLCH)₂ of the subject HILBA, the two binding domains are two single chain variable fragments (scFvs). Regardless of the type of binding domain, each binding domain includes a variable heavy chain domain (VH) and a variable light chain domain (VL).

In the variable region, three loops are gathered for each of the V domains of the heavy chain and light chain (VH and VL) to form an antigen-binding site. Each of the loops is referred to as a complementarity-determining region (hereinafter referred to as a “CDR”), in which the variation in the amino acid sequence is most significant. “Variable” refers to the fact that certain segments of the variable region differ extensively in sequence among antibodies. Variability within the variable region is not evenly distributed. Instead, the V regions consist of relatively invariant stretches called framework regions (FRs) of 15-30 amino acids separated by shorter regions of extreme variability called “hypervariable regions” that are each 9-15 amino acids long or longer.

Each VH and VL is composed of three hypervariable regions (“complementary determining regions,” “CDRs”) and four FRs, arranged from amino-terminus to carboxy-terminus in the following order: FR1-CDR1-FR2-CDR2-FR3-CDR3-FR4.

The hypervariable region generally encompasses amino acid residues from about amino acid residues 24-34 (LCDR1; “L” denotes light chain), 50-56 (LCDR2) and 89-97 (LCDR3) in the light chain variable region and around about 31-35B (HCDR1; “H” denotes heavy chain), 50-65 (HCDR2), and 95-102 (HCDR3) in the heavy chain variable region; Kabat et al., SEQUENCES OF PROTEINS OF IMMUNOLOGICAL INTEREST, 5th Ed. Public Health Service, National Institutes of Health, Bethesda, Md. (1991) and/or those residues forming a hypervariable loop (e.g. residues 26-32 (LCDR1), 50-52 (LCDR2) and 91-96 (LCDR3) in the light chain variable region and 26-32 (HCDR1), 53-55 (HCDR2) and 96-101 (HCDR3) in the heavy chain variable region; Chothia and Lesk (1987) J. Mol. Biol. 196:901-917. Specific CDRs of the invention are described below.

As will be appreciated by those in the art, the exact numbering and placement of the CDRs can be different among different numbering systems. However, it should be understood that the disclosure of a variable heavy and/or variable light sequence includes the disclosure of the associated (inherent) CDRs. Accordingly, the disclosure of each variable heavy region is a disclosure of the vhCDRs (e.g. vhCDR1, vhCDR2 and vhCDR3) and the disclosure of each variable light region is a disclosure of the vlCDRs (e.g. vlCDR1, vlCDR2 and vlCDR3).

A useful comparison of CDR numbering is as below, see Lafranc et al., Dev. Comp. Immunol. 27(1):55-77 (2003):

TABLE 2

| | Kabat + Chothia | IMGT | Kabat | AbM | Chothia | Contact | Xencor |
|---|---|---|---|---|---|---|---|
| vhCDR1 | 26-35 | 27-38 | 31-35 | 26-35 | 26-32 | 30-35 | 27-35 |
| vhCDR2 | 50-65 | 56-65 | 50-65 | 50-58 | 52-56 | 47-58 | 54-61 |
| vhCDR3 | 95-102 | 105-117 | 95-102 | 95-102 | 95-102 | 93-101 | 103-116 |
| vlCDR1 | 24-34 | 27-38 | 24-34 | 24-34 | 24-34 | 30-36 | 27-38 |
| vlCDR2 | 50-56 | 56-65 | 50-56 | 50-56 | 50-56 | 46-55 | 56-62 |
| vlCDR3 | 89-97 | 105-117 | 89-97 | 89-97 | 89-97 | 89-96 | 97-105 |

Throughout the present specification, the Kabat numbering system is generally used when referring to a residue in the variable domain (approximately, residues 1-107 of the light chain variable region and residues 1-113 of the heavy chain variable region) and the EU numbering system for Fc regions (e.g., Kabat et al., supra (1991)).

The present invention provides a large number of different CDR sets. In this case, a “full CDR set” comprises the three variable light and three variable heavy CDRs, e.g. a vlCDR1, vlCDR2, vlCDR3, vhCDR1, vhCDR2 and vhCDR3. These can be part of a larger variable light or variable heavy domain, respectfully. In addition, as more fully outlined herein, the variable heavy and variable light domains can be on separate polypeptide chains, when a heavy and light chain is used (for example when Fabs are used), or on a single polypeptide chain in the case of scFv sequences.

The CDRs contribute to the formation of the antigen-binding, or more specifically, epitope binding site of antibodies. “Epitope” refers to a determinant that interacts with a specific antigen binding site in the variable region of an antibody molecule known as a paratope. Epitopes are groupings of molecules such as amino acids or sugar side chains and usually have specific structural characteristics, as well as specific charge characteristics. A single antigen may have more than one epitope.

The epitope may comprise amino acid residues directly involved in the binding (also called immunodominant component of the epitope) and other amino acid residues, which are not directly involved in the binding, such as amino acid residues which are effectively blocked by the specifically antigen binding peptide; in other words, the amino acid residue is within the footprint of the specifically antigen binding peptide.

Epitopes may be either conformational or linear. A conformational epitope is produced by spatially juxtaposed amino acids from different segments of the linear polypeptide chain. A linear epitope is one produced by adjacent amino acid residues in a polypeptide chain. Conformational and nonconformational epitopes may be distinguished in that the binding to the former but not the latter is lost in the presence of denaturing solvents.

An epitope typically includes at least 3, and more usually, at least 5 or 8-10 amino acids in a unique spatial conformation. Antibodies that recognize the same epitope can be verified in a simple immunoassay showing the ability of one antibody to block the binding of another antibody to a target antigen, for example “binning” As outlined below, the invention not only includes the enumerated antigen binding domains and antibodies herein, but those that compete for binding with the epitopes bound by the enumerated antigen binding domains.

In certain embodiments, the ABDs of the subject HILBAs comprise a heavy chain variable region with frameworks from a particular germline heavy chain immunoglobulin gene and/or a light chain variable region from a particular germline light chain immunoglobulin gene. For example, such ABDs may comprise or consist of a human ABD comprising heavy or light chain variable regions that are “the product of” or “derived from” a particular germline sequence. An ABD that is “the product of” or “derived from” a human germline immunoglobulin sequence can be identified as such by comparing the amino acid sequence of the ABD to the amino acid sequences of human germline immunoglobulins and selecting the human germline immunoglobulin sequence that is closest in sequence (i.e., greatest % identity) to the sequence of the ABD. An ABD that is “the product of” or “derived from” a particular human germline immunoglobulin sequence may contain amino acid differences as compared to the germline sequence, due to, for example, CDRs, naturally-occurring somatic mutations or intentional introduction of site-directed mutation. However, a humanized ABD typically is at least 90% identical in amino acids sequence to an amino acid sequence encoded by a human germline immunoglobulin gene and contains amino acid residues that identify the ABD as being derived from human sequences when compared to the germline immunoglobulin amino acid sequences of other species (e.g., murine germline sequences). In certain cases, a humanized ABD may be at least 95, 96, 97, 98 or 99%, or even at least 96%, 97%, 98%, or 99% identical in amino acid sequence to the amino acid sequence encoded by the germline immunoglobulin gene. Typically, a humanized ABD derived from a particular human germline sequence will display no more than 10-20 amino acid differences from the amino acid sequence encoded by the human germline immunoglobulin gene (prior to the introduction of any skew, pI and ablation variants herein; that is, the number of variants is generally low, prior to the introduction of the variants of the invention). In certain cases, the humanized ABD may display no more than 5, or even no more than 4, 3, 2, or 1 amino acid difference from the amino acid sequence encoded by the germline immunoglobulin gene (again, prior to the introduction of any skew, pI and ablation variants herein; that is, the number of variants is generally low, prior to the introduction of the variants of the invention). In one embodiment, the parent ABD has been affinity matured, as is known in the art. Structure-based methods may be employed for humanization and affinity maturation, for example as described in U.S. Ser. No. 11/004,590. Selection based methods may be employed to humanize and/or affinity mature antibody variable regions, including but not limited to methods described in Wu et al., 1999, J. Mol. Biol. 294:151-162; Baca et al., 1997, J. Biol. Chem. 272(16):10678-10684; Rosok et al., 1996, J. Biol. Chem. 271(37): 22611-22618; Rader et al., 1998, Proc. Natl. Acad. Sci. USA 95: 8910-8915; Krauss et al., 2003, Protein Engineering 16(10):753-759, all entirely incorporated by reference. Other humanization methods may involve the grafting of only parts of the CDRs, including but not limited to methods described in U.S. Ser. No. 09/810,510; Tan et al., 2002, J. Immunol. 169:1119-1125; De Pascalis et al., 2002, J. Immunol. 169:3076-3084, all entirely incorporated by reference.

1. CD3 Antigen Binding Domains

In some embodiments, the HILBA includes a CD3 antigen binding domain. With respect to the (scFv-scCLCH)₁ format (See FIG. 12A), the CD3 binding domain can be either the scFv or the Fab. In the (scFv-scCLCH)₂ format (FIG. 12B), the CD3 binding domain is either scFv1 or scFv2. In exemplary embodiments, wherein the HILBA is a (scFv-scCLCH)₁, the CD3 binding domain is the scFv. In other embodiments, wherein the HILBA is a (scFv-scCLCH)₁, the CD3 binding domain is the Fab.

In embodiments wherein the CD3 ABD is a scFv, the scFv includes VH and VL domains that are joined using an scFv linker, which can be optionally a charged scFv linker. As will be appreciated by those in the art, the scFv can be assembled from N- to C-terminus, as N-VH-scFv linker-VL-C or as N-VL-scFv linker-VH-C.

As will be appreciated by those in the art, suitable CD3 antigen binding domains can comprise a set of 6 CDRs as depicted in the sequence listing and figures (e.g., FIG. 11), either as they are underlined/bolded or, in the case where a different numbering scheme is used as described herein and as shown in Table 2, as the CDRs that are identified using other alignments within the variable heavy (VH) domain and variable light domain (VL) sequences of those depicted in the figures (e.g., FIG. 11) and the sequence listing. Suitable CD3 ABDs that find use in the subject fusion proteins can also include the entire VH and VL sequences as depicted in these sequences and figures, used as scFvs or as Fabs.

In one embodiment, the CD3 antigen binding domain includes the 6 CDRs (i.e., vhCDR1-3 and vlCDR1-3) of any of the CD3 binding domains described in FIG. 11 or the sequence listing.

In addition to the parental CDR sets disclosed in the figures and sequence listing that form an ABD to CD3, provided herein are variant CD3 ABDS having CDRs that include at least one modification of the CD3 ABD CDRs disclosed herein (e.g., FIG. 11). In one embodiment, the HILBA includes a CD3 ABD that includes a set of 6 CDRs with 1, 2, 3, 4, 5, 6, 7, 8, 9, 10 amino acid modifications as compared to the 6 CDRs of a CD3 ABD as depicted in FIG. 11 or the sequence listing. In certain embodiments, the CD3 ABD is capable of binding CD3 antigen, as measured by at least one of a Biacore, surface plasmon resonance (SPR) and/or BLI (biolayer interferometry, e.g., Octet assay) assay, with the latter finding particular use in many embodiments.

In one embodiment, the CD3 ABD of the subject HILBAs includes 6 CDRs that are at least 90, 95, 97, 98 or 99% identical to the 6 CDRs of a CD3 ABD as depicted in FIG. 11 or the sequence listing. In certain embodiments, the CD3 ABD is capable of binding to human CD3, as measured by at least one of a Biacore, surface plasmon resonance (SPR) and/or BLI (biolayer interferometry, e.g., Octet assay) assay, with the latter finding particular use in many embodiments. In exemplary embodiments, the CD3 ABD is the scFv of the HILBA.

In one embodiment of the subject heterodimeric fusion protein, the CD3 ABD includes the 6 CDRs (i.e., vhCDR1-3 and vlCDR1-3) of one of the following CD3 ABDs (FIG. 11): [anti-CD3]_H1.30_L1.47; [anti-CD3]_H1.32_L1.47; [anti-CD3]_H1.33_L1.47; [anti-CD3]_H1.89_L1.47; [anti-CD3]_H1.31_L1.47; and [anti-CD3]_H1.90_L1.47.

In one embodiment, the CD3 antigen binding domain is a variant CD3 antigen binding domain that includes 6 CDRs (i.e., vhCDR1-3 and vlCDR1-3), where the 6 CDRs include 1, 2, 3, 4, 5, 6, 7, 8, 9, or 10 modifications as compared to the 6 CDRs of one of the following CD3 ABDs (FIG. 11): [anti-CD3]_H1.30_L1.47; [anti-CD3]_H1.32_L1.47; [anti-CD3]_H1.33_L1.47; [anti-CD3]_H1.89_L1.47; [anti-CD3]_H1.31_L1.47; and [anti-CD3]_H1.90_L1.47.

In one embodiment, the CD3 antigen binding domain of the HILBA is a variant CD3 antigen binding domain that includes 6 CDRs (i.e., vhCDR1-3 and vlCDR1-3), where the 6 CDRs are at least 90, 95, 97, 98 or 99% identical as compared to the 6 CDRs of one of the following CD3 ABDs (FIG. 11): [anti-CD3]_H1.30_L1.47; [anti-CD3]_H1.32_L1.47; [anti-CD3]_H1.33_L1.47; [anti-CD3]_H1.89_L1.47; [anti-CD3]_H1.31_L1.47; and [anti-CD3]_H1.90_L1.47.

In some embodiments, the CD3 ABD of the heterodimeric fusion protein includes the variable heavy domain (VH) and variable light domain (VL) of any of the CD3 ABDs disclosed herein, including, but not limited to those disclosed in FIG. 11. In addition to the parental CD3 variable heavy and variable light domains disclosed herein, provided herein are subject heterodimeric fusion proteins having one or more CD3 ABDs that include a variable heavy domain and/or a variable light domain that are variants of a CD3 ABD VH and VL domain disclosed herein. In one embodiment, the variant VH domain and/or VL domain has from 1, 2, 3, 4, 5, 6, 7, 8, 9 or 10 amino acid changes from a VH and/or VL domain of a CD3 ABD depicted in FIG. 11 or the sequence listing. In certain embodiments, the CD3 ABD is capable of binding to human CD3, as measured at least one of a Biacore, surface plasmon resonance (SPR) and/or BLI (biolayer interferometry, e.g., Octet assay) assay, with the latter finding particular use in many embodiments.

In one embodiment, the variant VH and/or VL domain of the heterodimeric fusion protein is at least 90, 95, 97, 98 or 99% identical to the VH and/or VL of a CD3 ABD as depicted in FIG. 11 or the sequence listing. In certain embodiments, the CD3 ABD is capable of binding to CD3, as measured by at least one of a Biacore, surface plasmon resonance (SPR) and/or BLI (biolayer interferometry, e.g., Octet assay) assay, with the latter finding particular use in many embodiments.

In some embodiments, the CD3 ABD includes the VH and VL of a one of the following CD3 ABDs: [anti-CD3]_H1.30_L1.47; [anti-CD3]_H1.32_L1.47; [anti-CD3]_H1.33_L1.47; [anti-CD3]_H1.89_L1.47; [anti-CD3]_H1.31_L1.47; and [anti-CD3]_H1.90_L1.47.

In some embodiments, the CD3 ABD includes a VH and VL, where the VH and/or VL includes 1, 2, 3, 4, 5, 6, 7, 8, 9 or 10 amino acid modifications as compared to a VH and/or VL of one of the following CD3 ABDs: [anti-CD3]_H1.30_L1.47; [anti-CD3]_H1.32_L1.47; [anti-CD3]_H1.33_L1.47; [anti-CD3]_H1.89_L1.47; [anti-CD3]_H1.31_L1.47; and [anti-CD3]_H1.90_L1.47.

In certain embodiments, the CD3 ABD includes a VH and VL, where the VH and VL are at least 90, 95, 97, 98 or 99% identical as compared to a VH and VL of one of the following CD3 ABDs (FIG. 11): [anti-CD3]_H1.30_L1.47; [anti-CD3]_H1.32_L1.47; [anti-CD3]_H1.33_L1.47; [anti-CD3]_H1.89_L1.47; [anti-CD3]_H1.31_L1.47; and [anti-CD3]_H1.90_L1.47.

2. Tumor Target Antigen Binding Domains

In some embodiments, the HILBAs provided herein include a tumor target antigen binding domain. Any suitable tumor target antigen binding domain can be included in the subject HILBAs provided herein including but are not limited to, CD20, CD38, CD123; ROR1, ROR2, BCMA; PSMA; SSTR2; SSTR5, CD19, FLT3, CD33, PSCA, ADAM 17, CEA, Her2, EGFR, EGFR-vIII, CD30, FOLR1, GD-2, CA-IX, Trop-2, CD70, CD38, mesothelin, EphA2, CD22, CD79b, GPNMB, CD56, CD138, CD52, CD74, CD30, CD123, RON, ERBB2, and EGFR. those described, for example, in U.S. Ser. No. 10/259,887B2, US20160229924A1, U.S. Pat. No. 9,822,186B2, and U.S. Ser. No. 10/316,088B2, all of which are expressly incorporated by reference in their entirety and particularly for the tumor target antigen binding domains disclosed therein.

### F. Exemplary Embodiments

In an exemplary embodiment, the HILBA is a (scFv-scCLCH)₁ format HILBA that includes a first monomer that includes, from N- to C-terminus: VH1-CH1-hinge-CH2-CH3, wherein VH1 is a first variable heavy domain and CH2-CH3 is a first Fc domain; b) a second monomer that includes, from N- to C-terminus: VL1-CL, wherein VL1 is a first variable light domain; and c) a third monomer that includes, from N- to C-terminus: VH2-scFv linker-VL2-CL-domain linker-CH1-hinge-CH2-CH3, wherein CH2-CH3 is a second Fc domain. In such an embodiment, VH1 and VL1 form an antigen binding domain that binds a first antigen and VH2-scFv linker-VL2 is a CD3 binding domain. Further in such embodiments the heavy chain constant domain (i.e., CH1-hinge-CH2-CH3) of the first monomer includes pI variants N208D/Q295E/N384D/Q418E/N421D, heterodimerization skew variants L368D/K370S, and FcKO variants E233P/L234V/L235A/G236del/S267K, the heavy chain constant domain (i.e., CH1-hinge-CH2-CH3) of the second monomer includes heterodimerization skew variants S364K/E37Q and FcKO variants E233P/L234V/L235A/G236del/S267K, wherein numbering is according to EU numbering. In some embodiments, each of the first and second Fc domains include half-life extension variants M428L/N434S, wherein numbering is according to EU numbering. In some embodiments, VH2 and VL2 have the sequences of the VH and VL of any of the following CD3 ABDs: [anti-CD3]_H1.30_L1.47; [anti-CD3]_H1.32_L1.47; [anti-CD3]_H1.33_L1.47; [anti-CD3]_H1.89_L1.47; [anti-CD3]_H1.31_L1.47; and [anti-CD3]_H1.90_L1.47. In an exemplary embodiment, VH1 and VL1 form a tumor target antigen binding domain. In some embodiments, VH1 and VL1 for a tumor antigen binding domain. In particular embodiments, the tumor target antigen binding domain binds CD20, CD38, CD123; ROR1, ROR2, BCMA; PSMA; SSTR2; SSTR5, CD19, FLT3, CD33, PSCA, ADAM 17, CEA, Her2, EGFR, EGFR-vIII, CD30, FOLR1, GD-2, CA-IX, Trop-2, CD70, CD38, mesothelin, EphA2, CD22, CD79b, GPNMB, CD56, CD138, CD52, CD74, CD30, CD123, RON, ERBB2, or EGFR. Exemplary (scFv-scCLCH)₁ format HILBAs are depicted in FIGS. 13A-C, 22A-I and 30A-C.

In some embodiments, VH2 and CH1 in the third monomer each include an engineered cysteine residue, wherein the engineered cysteine residues form a disulfide bond that stabilizes the third monomer (dsHC variants). In some embodiments, the engineered cysteine residue in VH2 is amino acid substitution S112C or S113C, wherein numbering is according to Kabat numbering (see Table 1), and the engineered cysteine residue in CH1 in the third monomer is A118C, wherein numbering is according to EU numbering (or A114C according to Kabat). Exemplary (scFv-scCLCH)₁ format antibodies with the dsHC variants are depicted in FIGS. 22A-I.

In some embodiments, the VH2 and VL2 each include an engineered cysteine residue, wherein the engineered cysteine residues form a disulfide bond that stabilizes the scFv (dsscFv variants). In some embodiments, the engineered cysteine residue in VH2 is amino acid substitution G44C and the engineered cysteine residue in VL2 is G100C, wherein numbering is according to Kabat numbering (see Table 1). Exemplary (scFv-scCLCH)₁ format antibodies with the dsscFv variants are depicted in FIGS. 30A-C.

In an exemplary embodiment, the third monomer includes both dsscFv variants and dsHC variants.

### IV. Nucleic Acids

In another aspect, provided herein are nucleic acid compositions encoding the subject heterodimeric IgG-like bispecific antibodies (HILBAs) and heterodimeric proteins described herein. As will be appreciated by those in the art, the nucleic acid compositions will depend on the format of the HILBA. Thus, for example, when the format requires two monomers ((scFv-scCLCH)₂ format HILBAs), two nucleic acid sequences can be incorporated into one or more expression vectors for expression. Similarly, for some formats, three nucleic acids are needed (e.g., ((scFv-scCLCH)₁ format HILBAs), which can be put into one expression vectors.

As is known in the art, the nucleic acids encoding the monomer components of heterodimeric IgG-like bispecific antibodies (HILBAs) and heterodimeric proteins can be incorporated into expression vectors as is known in the art, and depending on the host cells used to produce the fusion proteins. Generally, the nucleic acids are operably linked to any number of regulatory elements (promoters, origin of replication, selectable markers, ribosomal binding sites, inducers, etc.). The expression vectors can be extra-chromosomal or integrating vectors.

The nucleic acids and/or expression vectors are then transformed into any number of different types of host cells as is well known in the art, including, but not limited to, mammalian, bacterial, yeast, insect and/or fungal cells, with mammalian cells (e.g. CHO cells) being preferred.

In some embodiments, nucleic acids encoding each monomer are each contained within a single expression vector, generally under different or the same promoter controls. In certain embodiments, each of the nucleic acids are contained on a different expression vector.

The subject heterodimeric IgG-like bispecific antibodies (HILBAs) and heterodimeric proteins are made by culturing host cells comprising the expression vector(s) as is well known in the art. Once produced, traditional fusion protein or antibody purification steps are done, including an ion exchange chromatography step. As discussed herein, having the pIs of the two monomers differ by at least 0.5 can allow separation by ion exchange chromatography or isoelectric focusing, or other methods sensitive to isoelectric point. That is, the inclusion of pI variants that alter the isoelectric point (pI) of each monomer so that each monomer has a different pI and the resulting HILBAs has a distinct pI advantageously facilitates isoelectric purification of the heterodimer (e.g., anionic exchange chromatography, cationic exchange chromatography). These substitutions also aid in the determination and monitoring of any contaminating homodimers post-purification (e.g., IEF gels, cIEF, and analytical IEX columns).

### V. Biological and Biochemical Functionality of Heterodimeric IgG-Like Bispecific Antibodies

Subject heterodimeric IgG-like bispecific antibodies (HILBAs) that are useful for the treatment of cancers as described herein are administered to patients with cancer, and efficacy is assessed, in a number of ways as described herein. Thus, while standard assays of efficacy can be run, such as cancer load, size of tumor, evaluation of presence or extent of metastasis, etc., immuno-oncology treatments can be assessed on the basis of immune status evaluations as well. This can be done in a number of ways, including both in vitro and in vivo assays.

A. Fusion Protein Compositions for In Vivo Administration

Formulations of the heterodimeric IgG-like bispecific antibodies and heterodimeric proteins described herein are prepared for storage by mixing an antibody having the desired degree of purity with optional pharmaceutically acceptable carriers, excipients or stabilizers (Remington's Pharmaceutical Sciences 16th edition, Osol, A. Ed. [1980]), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

B. Administrative Modalities

The heterodimeric IgG-like bispecific antibodies and chemotherapeutic agents are administered to a subject, in accord with known methods, such as intravenous administration as a bolus or by continuous infusion over a period of time.

C. Treatment Modalities

In the methods of treatment provided herein, therapy is used to provide a positive therapeutic response with respect to a disease or condition (e.g., a cancer). By “positive therapeutic response” is intended an improvement in the disease or condition, and/or an improvement in the symptoms associated with the disease or condition. For example, a positive therapeutic response would refer to one or more of the following improvements in the disease: (1) a reduction in the number of neoplastic cells; (2) an increase in neoplastic cell death; (3) inhibition of neoplastic cell survival; (5) inhibition (i.e., slowing to some extent, preferably halting) of tumor growth; (6) an increased patient survival rate; and (7) some relief from one or more symptoms associated with the disease or condition.

Positive therapeutic responses in any given disease or condition can be determined by standardized response criteria specific to that disease or condition. Tumor response can be assessed for changes in tumor morphology (i.e., overall tumor burden, tumor size, and the like) using screening techniques such as magnetic resonance imaging (MRI) scan, x-radiographic imaging, computed tomographic (CT) scan, bone scan imaging, endoscopy, and tumor biopsy sampling including bone marrow aspiration (BMA) and counting of tumor cells in the circulation.

In addition to these positive therapeutic responses, the subject undergoing therapy may experience the beneficial effect of an improvement in the symptoms associated with the disease.

Treatment according to the present invention includes a “therapeutically effective amount” of the medicaments used. A “therapeutically effective amount” refers to an amount effective, at dosages and for periods of time necessary, to achieve a desired therapeutic result.

A therapeutically effective amount may vary according to factors such as the disease state, age, sex, and weight of the individual, and the ability of the medicaments to elicit a desired response in the individual. A therapeutically effective amount is also one in which any toxic or detrimental effects of the protein or protein portion are outweighed by the therapeutically beneficial effects.

A “therapeutically effective amount” for tumor therapy may also be measured by its ability to stabilize the progression of disease. The ability of a compound to inhibit cancer may be evaluated in an animal model system predictive of efficacy in human tumors.

Alternatively, this property of a composition may be evaluated by examining the ability of the compound to inhibit cell growth or to induce apoptosis by in vitro assays known to the skilled practitioner. A therapeutically effective amount of a therapeutic compound may decrease tumor size, or otherwise ameliorate symptoms in a subject. One of ordinary skill in the art would be able to determine such amounts based on such factors as the subject's size, the severity of the subject's symptoms, and the particular composition or route of administration selected.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. Parenteral compositions may be formulated in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

The specification for the dosage unit forms of the present invention are dictated by and directly dependent on (a) the unique characteristics of the active compound and the particular therapeutic effect to be achieved, and (b) the limitations inherent in the art of compounding such an active compound for the treatment of sensitivity in individuals.

The efficient dosages and the dosage regimens for the heterodimeric IgG-like bispecific antibodies used in the present invention depend on the disease or condition to be treated and may be determined by the persons skilled in the art.

An exemplary, non-limiting range for a therapeutically effective amount of an heterodimeric proteins used in the present invention is about 0.1-100 mg/kg.

All cited references are herein expressly incorporated by reference in their entirety.

Whereas particular embodiments of the invention have been described above for purposes of illustration, it will be appreciated by those skilled in the art that numerous variations of the details may be made without departing from the invention as described in the appended claims.

### EXAMPLES

Examples are provided below to illustrate the present invention. These examples are not meant to constrain the present invention to any particular application or theory of operation. For all constant region positions discussed in the present invention, numbering is according to the EU index as in Kabat (Kabat et al., 1991, Sequences of Proteins of Immunological Interest, 5th Ed., United States Public Health Service, National Institutes of Health, Bethesda, entirely incorporated by reference). Those skilled in the art of antibodies will appreciate that this convention consists of nonsequential numbering in specific regions of an immunoglobulin sequence, enabling a normalized reference to conserved positions in immunoglobulin families. Accordingly, the positions of any given immunoglobulin as defined by the EU index will not necessarily correspond to its sequential sequence.

General and specific scientific techniques are outlined in US Publ. App. No. 2015/0307629, US Publ. App. No. 2014/0288275, U.S. Pat. No. 9,605,084 and WO 2014/145806, all of which are expressly incorporated by reference in their entirety and particularly for the techniques outlined therein.

### Example 1: Heterodimeric IgG-Like Bispecific Antibodies (HILA)

In order to obtain a bispecific antibody structurally similar to an intact IgG, we envisioned the Heterodimeric IgG-Like Bispecific Antibody formats. One such HILBA format engineered as a proof of principle was (scFv-scCLCH)₁ (illustrative cartoon schematic depicted in FIG. 12A) which comprises a first monomer having a first variable heavy region (VH1) covalently attached to a first constant heavy domain comprising a first Fc domain, a second monomer comprising a first variable light region (VL1) covalently attached to a first constant light domain, and a third monomer comprising a single-chain variable region (scFv) covalently attached to a single-chain constant light/constant heavy domain (scCLCH), wherein said scFv comprises a second variable heavy region (VH2) covalently attached via a linker to a second variable light region (VL2), and wherein said scCLCH comprises a second constant light domain covalently attached via a domain linker to a second constant heavy domain comprising a second Fc domain.

Sequences for illustrative αCD123×αCD3 bispecific antibodies in the (scFv-scCLCH)₁ format are depicted in FIG. 13. Here, we describe the production and characterization of such a bispecific antibody (XENP28853).

1A: Production and Purification of Bispecific Antibodies in the HILBA Format.

Plasmids coding for the variable heavy and variable light regions of an illustrative anti-CD123 ABD were constructed by standard gene synthesis, followed by subcloning into a pTT5 expression vector containing appropriate fusion partners (e.g., constant heavy domain monomer l(−) and constant light domain—kappa as depicted in FIGS. 7 and 8); and plasmids coding for an scFv with the variable heavy and variable light regions of an illustrative anti-CD3 ABD was constructed by standard gene synthesis, followed by subcloning into a pTT5 expression vector containing appropriate scCLCH fusion partner (e.g., scCL(lambda)CH as depicted in FIG. 9).

Proteins were produced by transient transfection in HEK293E cells and were purified by a two-step purification process comprising protein A chromatography (purification part 1) followed by cation exchange chromatography (purification part 2). Chromatogram from purification part 2 for illustrative αCD123×αCD3 bispecific in the HILBA format (XENP28853) is depicted in FIG. 14. The chromatogram shows the isolation of three peaks (peak A, peak B, and peak BC), which were further characterized by analytical size-exclusion chromatography with multi-angle light scattering (aSEC-MALS) and analytical cation-exchange chromatography (aCIEX) for identity, purity and homogeneity as generally described below.

Peaks A, B, and BC isolated from purification part 2 for XENP28853 were analyzed using aSEC-MALS to deduce their component protein species. The analysis was performed on an AGILENT 1200 high-performance liquid chromatography (HPLC) system. Samples were injected onto a SUPERDEX™ 200 10/300 GL column (GE Healthcare Life Sciences) at 1.0 mL/min using 1×PBS, pH 7.4 as the mobile phase at 4° C. for 25 minutes with UV detection wavelength at 280 nM. MALS was performed on a miniDAWN® TREOS® with an Optilab® T-rEX Refractive Index Detector (Wyatt Technology, Santa Barbara, Calif.). Analysis was performed using Agilent OpenLab Chromatography Data System (CDS) ChemStation Edition AIC version C.01.07 and ASTRA version 6.1.7.15. Chromatograms depicting aSEC separation profiles for peaks A, B, and BC are depicted in FIG. 15 along with molecular weights of component protein species as determined by MALS. The profiles show that peak A comprises a dominant species having a MW of 148.2 kDa, as well as a species having a MW of 290.5 kDa; peak B comprises a dominant species having a MW of 147.0 kDa; and peak BC comprises a dominant species having a MW of 318.8 kDa, as well as species having MW of 221.3 kDa and 188.8 kDa. The calculated molecular weight of XENP28853 (anti-CD123×anti-CD3 species) is 149.3 kDa, while the calculated molecular weight of the anti-CD123×anti-CD123 species is 147.2 kDa. Accordingly, it was not possible to determine which peak contained XENP28853 based solely on the molecular weights of dominant species in peaks A and B as determined by MALS.

However, XENP28853 has a calculated pI of 8.69, while the anti-CD123×anti-CD123 species has a lower calculate pI of 7.88. Accordingly, Peaks A and B from purification part 2 were also analyzed using aCIEX to confirm which peak comprised XENP28853, as well as to assess purity and homogeneity. The analysis was performed on an Agilent 1200 high-performance liquid chromatography (HPLC) system. Samples were injected onto a Proteomix SCX-NP5 5 μM non-porous column (Sepax Technologies, Inc., Newark, Del.) at 1.0 mL/min using 0-40% NaCl gradient in 20 mM MES, pH 6.0 buffer with UV detection wavelength at 280 nM. Analysis was performed using Agilent OpenLAB CDS ChemStation Edition AIC version C.01.07. Chromatograms depicting aCIEX separation of peaks A and B are depicted in FIG. 16. The separation shows earlier elution of species from Peak A than species from Peak B, indicating that Peak B comprises XENP28853 which has the higher calculated pI. From hereon, XENP28853 refers to peak B as isolated from purification part 2 as depicted in FIG. 14.

1B: Antigen Binding by Bispecific Antibodies in the HILBA Format

Next, we investigated the binding of prototype HILBA XENP28853 to its antigens (CD3 and CD123) in comparison to a corresponding 1+1 Fab-scFv-Fc format bispecific antibody XENP27982 (sequences depicted in FIG. 10) with ABDs comprising the same variable regions as XENP28853 using Octet, a BioLayer Interferometry (BLI)-based method. Experimental steps for Octet generally including the following: Immobilization (capture of ligand to a biosensor); Association (dipping of ligand-coated biosensors into wells containing serial dilutions of the analyte); and Dissociation (returning of biosensors to well containing buffer) in order to determine the affinity of the test articles. A reference well containing buffer alone was also included in the method for background correction during data processing. In particular to assess binding to CD3, HIS1K biosensors were used to capture CD3Dε-Fc-His (Sino Biological, Wayne, Pa.) and dipped into multiple concentrations of XENP28853 or XENP27982; and to assess binding to CD123, anti-human Fc (AHC) biosensors were used to capture either XENP28853 or XENP27982 and dipped into multiple concentrations of recombinant CD123 (R&D Systems, Minneapolis, Minn.). Sensorgrams depicted in FIGS. 17-18 show that XENP28853 demonstrated similar binding profiles to CD3 and CD123 in comparison to XENP27982. Notably, this further confirms that peak B as isolated from purification part 2 is XENP28853.

1C: Stability of Bispecific Antibodies in the HILBA Format

Stability of prototype HILBA format XENP28853 was assessed in comparison to exemplary 1+1 Fab-scFv-Fc format bispecific antibodies XENP23535 and XENP13677 (sequences depicted in FIG. 10) using Differential Scanning Fluorimetry (DSF). DSF experiments were performed using a Bio-Rad CFX Connect Real-Time PCR Detection System. Proteins were mixed with SYPRO Orange fluorescent dye and diluted to 0.2 mg/mL in PBS. The final concentration of SYPRO Orange was 10×. After an initial 10 minute incubation period at 25° C., proteins were heated from 25 to 95° C. using a heating rate of 1° C./min. A fluorescence measurement was taken every 30 sec. Melting temperatures (Tm) were calculated using the instrument software. The stability result and corresponding melting curves are depicted in FIG. 19, and show that XENP28853 exhibits a first unfolding transition (at 66° C.) comparable to that of XENP23535 and XENP13677 (at 67° C.).

To further assess stability, prototype HILBA format XENP28853, at either 1.8 mg/ml or 18 mg/ml concentration, were heat stressed by incubation at 40° C. for 7 and 14 days. Following incubation, samples were assessed by aSEC and aCIEX as described above in Example 1A. Chromatograms depicting aSEC separation in FIG. 15 shows that minimal high molecular weight species (HMWS) indicative of aggregates resulted from heat stress at both low and high concentrations of XENP28853. Chromatograms depicting aCIEX separation in FIG. 16 show that acidic species do result from heat stress at both low and high concentrations, with more acidic species resulting from longer duration of heat stress.

### Example 2: Engineering the HILBA Format with Disulfide Stabilized Heavy Chain

The HILBA format described in Example 1 is much more structurally similar to an intact IgG than, for example, the 1+1 Fab-scFv-Fc format, in having CL and CH1 regions on both sides of the bispecific antibody and in view of the covalent attachment of the VL to the CL. However, in the HILBA format, the VH of the scFv(s) is not covalently attached to the CH1. Accordingly, to improve the stability of the HILBA format and to further emulate the intact IgG structure, we engineered disulfide bonds between the VH of the scFv(s) and the corresponding CH1. One such format we engineered as a proof of principle was (scFv-scCLCH)₁ with disulfide stabilized heavy chain (or (scFv-scCLCH)₁(dsHC); illustrative cartoon schematic depicted in FIG. 20A) which comprises a first monomer comprising a first variable heavy region (VH1) covalently attached to a first constant heavy domain, a second monomer comprising a first variable light region (VL1) covalently attached to a first constant light domain, and a third monomer comprising a single-chain variable region (scFv) covalently attached to a single-chain constant light/constant heavy domain (scCLCH), wherein said scFv comprises a second variable heavy region (VH2) covalently attached via an scFv linker to a second variable light region (VL2), and wherein said scCLCH comprises a second constant light domain covalently attached via a domain linker to a second constant heavy domain; and further wherein the C-terminus of VH2 comprises a first engineered cysteine residue, and the N-terminus of the second constant heavy domain comprises a second engineered cysteine residue so that the first and second engineered cysteine residues form a disulfide bond.

While the first engineered cysteine residue may be anywhere on the C-terminus of the second variable heavy region (VH2) so as to form a disulfide bond with a second engineered cysteine residue on the N-terminus of the second constant heavy domain, examples of such a substitution include, but are not limited to, S112C and S113C (numbering according to Kabat). Further, while the second engineered cysteine residue may be anywhere on the N-terminus of the second constant heavy domain so as to form a disulfide bond with the first engineered cysteine residue on the C-terminus of the second variable heavy region, examples of such substitutions include, but are not limited to, A114C (numbering according to Kabat).

Sequences for illustrative αCD123×αCD3, αCD20×αCD3, αPSMA×αCD3, and αSSTR2×αCD3 bispecific antibodies in the (scFv-scCLCH)₁(dsHC) format are depicted in FIG. 22.

1A: Production and Purification

Plasmids coding for the variable heavy and variable light regions of an illustrative anti-CD123 ABD were constructed by standard gene synthesis, followed by subcloning into a pTT5 expression vector containing appropriate fusion partners (e.g., constant heavy domain monomer l(−) and constant light domain—kappa as depicted in FIGS. 7 and 8); and plasmids coding for an scFv with the variable heavy and variable light regions of an illustrative anti-CD3 ABD (with 112C or 113C substitution in the variable heavy domain according to Kabat numbering) was constructed by standard gene synthesis, followed by subcloning into a pTT5 expression vector containing appropriate scCLCH fusion partner (e.g., scCL(lambda)CH(A118C) as depicted in FIG. 9).

Proteins were produced by transient transfection in HEK293E cells and were purified by a two-step purification process comprising protein A chromatography (purification part 1) followed by cation exchange chromatography (purification part 2). Chromatograms from purification part 2 for illustrative αCD123×αCD3 bispecific in the HILBA format (XENP29169 or XENP29170) are depicted respectively in FIGS. 23 and 26. The chromatogram shows the isolation of two peaks (peak A and peak B), which were further characterized by analytical size-exclusion chromatography with multi-angle light scattering (aSEC-MALS) and analytical cation-exchange chromatography (aCIEX) for identity, purity and homogeneity as generally described in Example 1A (and as depicted in FIGS. 24-25 and 27-28). Consistent with Example 1A, aCIEX separation indicated that the protein in peak B (for both XENP29169 and XENP29170) was the purified protein.

### Example 3: Engineering the HILBA Format with Disulfide Stabilized scFvs

To further stabilize the HILBA format, we engineered disulfide bonds between the VH and VL within the scFv(s). As a proof of principle, we engineered the (scFv-scCLCH)₁ format with disulfide stabilized scFv (or (scFv-scCLCH)₁(dsscFv); illustrative cartoon schematic depicted in FIG. 20B) which comprises a first monomer comprising a first variable heavy region (VH1) covalently attached to a first constant heavy domain, a second monomer comprising a first variable light region (VL1) covalently attached to a first constant light domain, and a third monomer comprising a single-chain variable region (scFv) covalently attached to a single-chain constant light/constant heavy domain (scCLCH), wherein said scFv comprises a second variable heavy region (VH2) covalently attached via an scFv linker to a second variable light region (VL2), and wherein said scCLCH comprises a second constant light domain covalently attached via a domain linker to a second constant heavy domain; and further wherein the VH2 comprises a first engineered cysteine residue, the VL2 comprises a second engineered cysteine residue so that the first and second engineered cysteine residues form a disulfide bond.

Any method of engineering cysteine residues into scFvs to introduce interchain disulfide bonds known in the art may be used, for instance: engineering a cysteine at position 44 in the variable heavy region and a cysteine at position 100 in the variable light region (according to Kabat numbering; Reiter et al. (1994) Biochemistry 33:5451-5459); engineering a cysteine at position 105 in the variable heavy region and a cysteine at position 43 in the variable light region (according to Kabat numbering; Jung et al. (1994) Proteins, Struc. Func. Genet., 19:35-47); engineering a cysteine at position 100b in the variable heavy region and a cysteine at position 49 in the variable light region (according to Kabat numbering; Glockshuber et al. (1990) Biochemistry 29:1362-1367); engineering a cysteine at position 100 in the variable heavy region and a cysteine at position 50 in the variable light region (according to Kabat numbering; Glockshuber et al. (1990) Biochemistry 29:1362-1367); and engineering a cysteine at position 101 in the variable heavy region and a cysteine at position 46 in the variable light region (according to Kabat numbering; Zhu et al. (1997) Prot. Sci. 6:781-788). Sequences for an illustrative αCD123×αCD3, αCD20×αCD3, αPSMA×αCD3, and αSSTR2×αCD3 bispecific antibody in the (scFv-scCLCH)₁(dsscFv) format are depicted in FIG. 30.

1A: Production and Purification

Plasmids coding for the variable heavy and variable light regions of an illustrative anti-CD123 ABD were constructed by standard gene synthesis, followed by subcloning into a pTT5 expression vector containing appropriate fusion partners (e.g., constant heavy domain monomer l(−) and constant light domain—kappa as depicted in FIGS. 7 and 8); and plasmids coding for disulfide-stabilized scFv with the variable heavy and variable light regions of an anti-CD3 ABD was constructed by standard gene synthesis, followed by subcloning into a pTT5 expression vector containing appropriate scCLCH fusion partner (e.g., scCL(lambda)CH as depicted in FIG. 9).

Proteins were produced by transient transfection in HEK293E cells and were purified by a two-step purification process comprising protein A chromatography (purification part 1) followed by cation exchange chromatography (purification part 2). Chromatogram from purification part 2 for illustrative αCD123×αCD3 bispecific in the HILBA Format (XENP29171) is depicted in FIG. 31. The chromatogram shows the isolation of two peaks (peak A and peak B), which were further characterized by analytical size-exclusion chromatography with multi-angle light scattering (aSEC-MALS) and analytical cation-exchange chromatography (aCIEX) for identity, purity and homogeneity as generally described in Example 1A (and as depicted in FIGS. 32-33). Consistent with Example 1A and Example 2A, aCIEX separation indicated that the protein in peak B (from purification part 2 of XENP29171) was the purified protein.

### Example 4: Prototype CD3 Bispecifics in the HILBA Format are Active In Vitro

Next, we investigated redirected T cell cytotoxicity (RTCC) by the prototype HILBA format bispecific antibody XENP28853 in comparison to a 1+1 Fab-scFv-Fc format bispecific antibody XENP27982. KG1a cells, which express CD123, were incubated with T cells purified from human PBMCs and the indicated test articles at 37° C. for 24 hours at an effector:target ratio of 10:1. RTCC was determined using CytoTox-ONE™ Homogeneous Membrane Integrity Assay (Promega, Madison, Wis.) to measure lactate dehydrogenase levels according to manufacturer's instructions and data was acquired on a Wallac Victor2 Microplate Reader (PerkinElmer, Waltham, Mass.).

The data depicted in FIG. 34 show that both XENP28853 and XENP27982 induced killing of KG1a cells, although XENP28853 was less potent than XENP27982. Without wishing to be bound by theory, it is hypothesized that the different geometry of the CD3 binding domain in the HILBA format and in the 1+1 Fab-scFv-Fc format impact on antigen binding by the test articles leading to the difference in potency. Accordingly, it may be necessary to fine-tune the affinity of antigen binding domains in the context of the HILBA format to achieve equivalent potency.

### Example 5: Prototype CD3 Bispecifics in the HILBA Format Demonstrate Good Pharmacokinetics

One of the motivations for engineering bispecific antibodies structurally similar to the native IgG was to emulate its pharmacokinetic properties. Accordingly, we investigated the pharmacokinetics of prototype HILBA format bispecific antibody XENP28853 in C57BL/6J mice (The Jackson Laboratory, Bar Harbor, Me.). Mice (n=5) were dosed with 2 mg/kg of XENP28853 on Day 0, and blood was drawn on Day 0 (1 hour post dose), and Days 1, 4, 7, 11, 14, 18, and 21 to investigate serum concentration of XENP28853. Serum concentration of XENP28853 was determined by anti-human Fc capture and anti-human kappa detection. PK interpretative analysis was performed using Phoenix WinNonlin software (Version 6.4.0.768) with PK parameters for non-compartmental analysis of free drug serum concentration versus time. Pharmacokinetic profile of XENP28553 is depicted in FIG. 35, and half-life estimation based on the average serum concentration and data from Day 4 to 21 was 13.5 days. The half-life of native IgG is roughly 10-21 days (depending on attributes such as the variable regions), so the data from this study suggests that bispecific antibodies in the HILBA Format demonstrate good pharmacokinetics comparable to native IgG.

1.-12. (canceled)

13. A bispecific antibody comprising:
a) a first monomer comprising VH1-CH1-hinge-CH2-CH3, wherein VH1 is a first variable heavy domain and CH2-CH3 is a first variant Fc domain;
b) a second monomer comprising VL1-CL, wherein VL1 is a first variable light domain; and
c) a third monomer comprising VH2-scFv linker-VL2-CL-domain linker-CH1-hinge-CH2-CH3, wherein VH2 is a second variable domain, VL2 is a second variable light domain, and CH2-CH3 is a second variant Fc domain,
wherein VH1 and VL1 form a first antigen binding domain,
wherein VH2 and VL2 form a second antigen binding domain that binds a different antigen than the first antigen binding domain,
wherein the first variant Fc domain comprises skew variants L368D/K370S and the second variant Fc domain comprises skew variants S364K/E357Q,
wherein the first variant Fc domain and second variant Fc domain each comprise FcKO variants E233P/L234V/L235A/G236del/S267K, and
wherein the first variant Fc domain or second variant Fc domain comprises pI variants Q295E/N384D/Q418E/N421D.

14.-16. (canceled)

17. A nucleic acid composition comprising:
a) a first nucleic acid encoding the first monomer of claim 13;
b) a second nucleic acid encoding the second monomer of claim 13; and
c) a third nucleic acid encoding the third monomer of claim 13.

18. An expression vector composition comprising:
a) a first expression vector comprising the first nucleic acid of claim 17;
b) a second expression vector comprising the second nucleic acid of claim 17; and
c) a third expression vector comprising the third nucleic acid of claim 17.

19. A host cell comprising the expression vector composition of claim 18.

20. A method of making a bispecific antibody comprising culturing the host cell of claim 19 under conditions wherein the bispecific antibody is expressed, and recovering the antibody.

21.-32. (canceled)

33. A bispecific antibody comprising:
a) a first monomer comprising VH1-scFv linker-VL1-CL-domain linker-CH1-hinge-CH2-CH3, wherein VH1 is a first variable domain, VL1 is a first variable light domain, and CH2-CH3 is a first Fc domain; and
b) a second monomer comprising VH2-scFv linker-VL2-CL-domain linker-CH1-hinge-CH2-CH3, wherein VH2 is a second variable domain, VL2 is a second variable light domain, and CH2-CH3 is a second Fc domain,
wherein VH1 and VL1 form a first antigen binding domain,
wherein VH2 and VL2 form a second antigen binding domain that binds a different antigen than the first antigen binding domain,
wherein the first variant Fc domain comprises skew variants L368D/K370S and the second variant Fc domain comprises skew variants S364K/E357Q,
wherein the first variant Fc domain and second variant Fc domain each comprise FcKO variants E233P/L234V/L235A/G236del/S267K, and
wherein the first variant Fc domain or second variant Fc domain comprises pI variants Q295E/N384D/Q418E/N421D.

34.-52. (canceled)

53. A heterodimeric protein comprising:
a) a first monomer comprising a CH2-CH3, wherein CH2-CH3 is a first variant Fc domain; and
b) a second monomer comprising a VH-scFv linker-VL-CL-domain linker-CH1-hinge-CH2-CH3, wherein VH is a variable domain, VL is a variable light domain, and CH2-CH3 is a second variant Fc domain,
wherein VH and VL form an antigen binding domain,
wherein the first variant Fc domain comprises skew variants L368D/K370S and the second variant Fc domain comprises skew variants S364K/E357Q,
wherein the first variant Fc domain comprises FcKO variants E233P/L234V/L235A/G236del/S267K, and
wherein the first variant Fc domain or second variant Fc domain comprises pI variants Q295E/N384D/Q418E/N421D.

54.-60. (canceled)
